(19) United States
(12) Patent Application Publication
(10) Pub. No.: US 2021/0251994 A1
(43) Pub. Date: Aug. 19, 2021

(54) INCREASING IMMUNE ACTIVITY THROUGH MODULATION OF POSTCELLULAR SIGNALING FACTORS

(71) Applicant: FLAGSHIP PIONEERING INNOVATIONS V, INC., Cambridge, MA (US)

(72) Inventors: Anthony Michael Barsotti, New York, NY (US); Alexandra Masu Cantley, New York, NY (US); Jason Park, West Newton, MA (US)

(21) Appl. No.: 17/252,225

(22) PCT Filed: Jun. 14, 2019
(86) PCT No.: PCT/US2019/037350

§ 371 Date: Dec. 14, 2020

Related U.S. Application Data
(60) Provisional application No. 62/781,819, filed on Dec. 19, 2018.
(60) Provisional application No. 62/685,770, filed on Jun. 15, 2018.

(51) Int. Cl. A61K 31/517; G01N 33/50; A61K 31/4985; A61K 31/437; A61P 35/00; A61K 45/06

(52) U.S. Cl. CPC A61K 31/517; G01N 33/5047; G01N 33/5041; A61K 45/06; A61K 31/437; A61P 35/00; A61K 31/4985

(57) ABSTRACT

The invention provides methods of increasing immune response by inducing iron-dependent cellular disassembly. The increase in immune response may be used, for example, for treatment of infection or cancer. The invention also provides screening assays for identification of compounds that induce iron-dependent cellular disassembly and are also immunostimulatory agents. The invention further provides methods for identifying immunostimulatory agents produced by cells undergoing iron-dependent cellular disassembly.

### RELATED APPLICATIONS

This application claims priority to U.S. Provisional Patent Application No. 62/685,770 filed on Jun. 15, 2018, and U.S. Provisional Patent Application No. 62/781,819 filed on Dec. 19, 2018, the contents of each of which are incorporated by reference herein in their entirety.

### BACKGROUND

In multicellular organisms, cell death is a critical and active process that is believed to maintain tissue homeostasis and eliminate potentially harmful cells.

### SUMMARY OF THE INVENTION

In certain aspects, the disclosure relates to a method of increasing immune activity in an immune cell, comprising: (i) contacting a target cell with an agent that induces iron-dependent cellular disassembly and (ii) exposing the immune cell to the target cell that has been contacted with the agent or to postcellular signaling factors produced by the target cell that has been contacted with the agent, in an amount sufficient to increase the immune activity in the immune cell relative to an immune cell in the absence of contacting the target cell with the agent, wherein the agent that induces iron-dependent cellular disassembly is selected from the group consisting of an inhibitor of antiporter system Xc⁻, an inhibitor of GPX4, and a statin.

In certain aspects, the disclosure relates to a method of increasing the level or activity of NFkB in an immune cell, comprising: (i) contacting a target cell with an agent that induces iron-dependent cellular disassembly and (ii) exposing the immune cell to the target cell that has been contacted with the agent or to postcellular signaling factors produced by the target cell that has been contacted with the agent, in an amount sufficient to increase the level or activity of NFkB in the immune cell relative to an immune cell in the absence of contacting the target cell with the agent, wherein the agent that induces iron-dependent cellular disassembly is selected from the group consisting of an inhibitor of antiporter system Xc⁻, an inhibitor of GPX4, and a statin.

In certain aspects, the disclosure relates to a method of increasing the level or activity of interferon regulatory factor (IRF) or Stimulator of Interferon Genes (STING) in an immune cell, comprising: (i) contacting a target cell with an agent that induces iron-dependent cellular disassembly and (ii) exposing the immune cell to the target cell that has been contacted with the agent or to postcellular signaling factors produced by the target cell that has been contacted with the agent, in an amount sufficient to increase the level or activity of IRF or STING in the immune cell relative to an immune cell in the absence of contacting the target cell with the agent, wherein the agent that induces iron-dependent cellular disassembly is selected from the group consisting of an inhibitor of antiporter system Xc⁻, an inhibitor of GPX4, and a statin.

In certain aspects, the disclosure relates to a method of increasing the level or activity of a pro-immune cytokine in an immune cell, comprising: (i) contacting a target cell with an agent that induces iron-dependent cellular disassembly and (ii) exposing the immune cell to the target cell that has been contacted with the agent or to postcellular signaling factors produced by the target cell that has been contacted with the agent, in an amount sufficient to increase the level or activity of the pro-immune cytokine in the immune cell relative to an immune cell in the absence of contacting the target cell with the agent, wherein the agent that induces iron-dependent cellular disassembly is selected from the group consisting of an inhibitor of antiporter system Xc⁻, an inhibitor of GPX4, and a statin.

In certain embodiments, the iron-dependent cellular disassembly is ferroptosis. In certain embodiments, the inhibitor of antiporter system Xc⁻ is erastin or a derivative or analog thereof.

In certain embodiments, the erastin or derivative or analog thereof has the following formula:

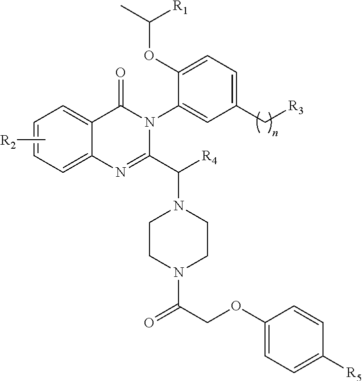

or pharmaceutically acceptable salts or esters thereof, wherein

R₁ is selected from the group consisting of H, C₁₋₄ alkyl, C₁₋₄ alkoxy, hydroxy, and halogen;

R₂ is selected from the group consisting of H, halo, and C₁₋₄ alkyl;

R₃ is selected from the group consisting of H, C₁₋₄ alkyl, C₁₋₄ alkoxy, 5-7 membered heterocycloalkyl, and 5-6 membered heteroaryl;

R₄ is selected from the group consisting of H and C₁₋₄ alkyl;

R₅ is halo;

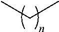

is optionally substituted with ═O; and
n is an integer from 0-4.

In certain embodiments, the analog of erastin is PE or IKE.

In certain embodiments, the inhibitor of GPX4 is selected from the group consisting of (1S,3R)-RSL3 or a derivative or analog thereof, ML162, DPI compound 7, DPI compound 10, DPI compound 12, DPI compound 13, DPI compound 17, DPI compound 18, DPI compound 19, FIN56, and FIN02.

In certain embodiments, the RSL3 derivative or analog is a compound represented by Structural Formula (I):

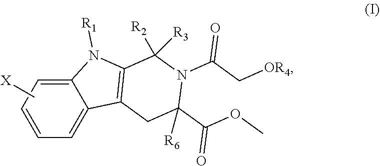

or an enantiomer, optical isomer, diastereomer, N-oxide, crystalline form, hydrate, or pharmaceutically acceptable salt thereof, wherein

R₁, R₂, R₃, and R₆ are independently selected from H, C₁₋₈alkyl, C₁₋₈alkoxy, C₁₋₈aralkyl, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkoxy, aralkyl, carbocyclic, heterocyclic, aryl, heteroaryl, acyl, alkylsulfonyl, and arylsulfonyl is optionally substituted with at least one substituent;

R₄ and R₅ are independently selected from H₁ C₁₋₈alkyl, C₁₋₈alkoxy, 3- to 8-membered carbocyclic, 3- to 8-membered heterocyclic, 3- to 8-membered aryl, or 3-to 8-membered heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether is optionally substituted with at least one substituent;

R⁷ is selected from H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;

R⁸ is selected from H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; and

X is 0-4 substituents on the ring to which it is attached.

In certain embodiments, the RSL3 derivative or analog is a compound represented by Structural Formula (II):

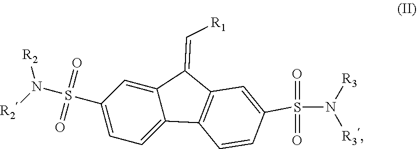

or an N-oxide, crystalline form, hydrate, or pharmaceutically acceptable salt thereof; wherein:

R₁ is selected from the group consisting of H, OH, and —(OCH₂CH₂)ₓOH;

X is an integer from 1 to 6; and

R₂, R₂′, R₃, and R₃′ independently are selected from the group consisting of H, C₃₋₈cycloalkyl, and combinations thereof, or R₂ and R₂′ may be joined together to form a pyridinyl or pyranyl and R₃ and R₃′ may be joined together to form a pyridinyl or pyranyl.

In certain embodiments, the RSL3 derivative or analog is a compound represented by Structural Formula (III):

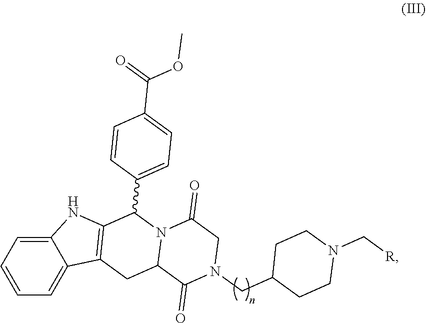

or a stereoisomer thereof, or a pharmaceutically acceptable salt thereof; wherein: n is 2, 3 or 4; and R is a substituted or unsubstituted C₁-C₆ alkyl group, a substituted or unsubstituted C₃-C₁₀ cycloalkyl group, a substituted or unsubstituted C₂-C₈ heterocycloalkyl group, a substituted or unsubstituted C₆-C₁₀ aromatic ring group, or a substituted or unsubstituted C₃-C₈ heteroaryl ring group; wherein the substitution means that one or more hydrogen atoms in each group are substituted by the following groups selected from the group consisting of: halogen, cyano, nitro, hydroxy, C₁-C₆ alkyl, halogenated C₁-C₆ alkyl, C₁-C₆ alkoxy, halogenated C₁-C₆ alkoxy, COOH (carboxy), COOC₁-C₆ alkyl, OCOC₁-C₆ alkyl.

In certain embodiments, the statin is selected from the group consisting of atorvastatin, fluvastatin, lovastatin, pitavastatin, pravastatin, rosuvastatin, cerivastatin and simvastatin. In certain embodiments, the immune cell is a macrophage, monocyte, dendritic cell, T cell, CD4+ cell, CD8+ cell, or CD3+ cell. In certain embodiments, the immune cell is a THP-1 cell.

In certain embodiments, the method is carried out in vitro or ex vivo. In certain embodiments, the method is carried out in vivo. In certain embodiments, step (i) is carried out in vitro and step (ii) is carried out in vivo.

In certain aspects, the disclosure relates to a method of increasing immune activity in a cell, tissue or subject, the method comprising administering to the cell, tissue or subject an agent that induces iron-dependent cellular disassembly in an amount sufficient to increase the immune activity relative to a cell, tissue or subject that is not treated with the agent that induces iron-dependent cellular disassembly.

In one embodiment, the subject is in need of an increased immune activity.

In one embodiment, the agent that induces iron-dependent cellular disassembly is administered in an amount sufficient to increase in the cell, tissue or subject one or more of: the level or activity of NFkB, the level or activity of IRF or STING, the level or activity of macrophages, the level or activity of monocytes, the level or activity of dendritic cells, the level or activity of T cells, the level or activity of CD4+, CD8+ or CD3+ cells, and the level or activity of a pro-immune cytokine.

In certain aspects, the disclosure relates to a method of increasing the level or activity of NFkB in a cell, tissue or subject, comprising administering to the cell, tissue or subject an agent that induces iron-dependent cellular disassembly in an amount sufficient to increase the level or activity of NFkB relative to a cell, tissue or subject that is not treated with the agent that induces iron-dependent cellular disassembly.

In one embodiment, the subject is in need of an increased level or activity of NFkB.

In one embodiment, the level or activity of NFkB is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In certain aspects, the disclosure relates to a method of increasing the level or activity of IRF or STING in a cell, tissue or subject, comprising administering to the cell, tissue or subject an agent that induces iron-dependent cellular disassembly in an amount sufficient to increase the level or activity of IRF or STING relative to a cell, tissue or subject that is not treated with the agent that induces iron-dependent cellular disassembly.

In one embodiment, the subject is in need of an increased level or activity of IRF or STING.

In one embodiment, the level or activity of IRF or STING is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In certain aspects, the disclosure relates to a method of increasing the level or activity of macrophages, monocytes, dendritic cells or T cells in a tissue or subject, comprising administering to the tissue or subject an agent that induces iron-dependent cellular disassembly in an amount sufficient to increase the level or activity of macrophages, monocytes, dendritic cells or T cells relative to a tissue or subject that is not treated with the agent that induces iron-dependent cellular disassembly.

In one embodiment, the subject is in need of an increased level or activity of macrophages, monocytes, dendritic cells or T cells.

In one embodiment, the level or activity of macrophages, monocytes, dendritic cells, or T cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the agent that induces iron-dependent cellular disassembly.

In certain aspects, the disclosure relates to a method of increasing the level or activity of CD4+, CD8+, or CD3+ cells in a tissue or subject, comprising administering to the subject an agent that induces iron-dependent cellular disassembly in an amount sufficient to increase the level or activity of CD4+, CD8+, or CD3+ cells relative to a tissue or subject that is not treated with the agent that induces iron-dependent cellular disassembly.

In one embodiment, the subject is in need of an increased level or activity of CD4+, CD8+, or CD3+ cells.

In one embodiment, the level or activity of CD4+, CD8+, or CD3+ cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the agent that induces iron-dependent cellular disassembly.

In certain aspects, the disclosure relates to a method of increasing the level or activity of a pro-immune cytokine in a cell, tissue or subject, comprising administering to the cell, tissue or subject an agent that induces iron-dependent cellular disassembly in an amount sufficient to increase the level or activity of the pro-immune cytokine relative to a cell, tissue or subject that is not treated with the agent that induces iron-dependent cellular disassembly.

In one embodiment, the subject is in need of an increased level or activity of a pro-immune cytokine.

In one embodiment, the level or activity of the pro-immune cytokine is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In one embodiment, the pro-immune cytokine is selected from IFN-α, IL-1, IL-12, IL-18, IL-2, IL-15, IL-4, IL-6, TNF-α, IL-17 and GMCSF.

In one embodiment, the method further includes, before the administration, evaluating the cell, tissue or subject for one or more of: the level or activity of NFkB; the level or activity of macrophages; the level or activity of monocytes; the level or activity of dendritic cells; the level or activity of CD4+ cells, CD8+ cells, or CD3+ cells; the level or activity of T cells; and the level or activity of a pro-immune cytokine.

In one embodiment, the method further includes, after the administration, evaluating the cell, tissue or subject for one or more of: the level or activity of NFkB; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine.

In embodiments, the pro-immune cytokine is selected from IFN-α, IL-1, IL-12, IL-18, IL-2, IL-15, IL-4, IL-6, TNF-α, IL-17 and GMCSF.

In one embodiment, the subject has an infection.

In one embodiment, the infection is a chronic infection.

In embodiments, the chronic infection is selected from HIV infection, HCV infection, HBV infection, HPV infection, Hepatitis B infection, Hepatitis C infection, EBV infection, CMV infection, TB infection, and infection with a parasite.

In one embodiment, the cell or tissue is a cancer cell or cancerous tissue.

In one embodiment, the subject has been diagnosed with cancer.

In certain aspects, the disclosure relates to a method of treating a subject in need of increased immune activity, the method comprising administering to the subject an agent that induces iron-dependent cellular disassembly in an amount sufficient to increase the immune activity in the subject.

In one embodiment, the subject has a chronic infection.

In embodiments, the chronic infection is selected from HIV infection, HCV infection, HBV infection, HPV infection, Hepatitis B infection, Hepatitis C infection, EBV infection, CMV infection, TB infection, and infection with a parasite.

In one embodiment, the subject has cancer.

In embodiments, the cancer is selected from melanoma, renal cell carcinoma, non-small cell lung cancer, non-squamous cell lung cancer, urothelial carcinoma, Hodgkin's lymphoma, head and neck squamous cell carcinoma, hepatocellular carcinoma, colorectal cancer, gastric adenocarcinoma, gastric esophageal junction adenocarcinoma, and Merkel cell carcinoma.

In one embodiment, the iron-dependent cellular disassembly is ferroptosis.

In certain aspects, the disclosure relates to a method of treating a subject diagnosed with cancer, comprising administering to the subject, in combination (a) an immunotherapeutic and (b) an agent that induces iron-dependent cellular disassembly, thereby treating the cancer in the subject.

In one embodiment, the agent that induces iron-dependent cellular disassembly is administered to the subject in an amount effective to increase immune response in the subject.

In one embodiment, the immunotherapeutic is selected from the group consisting of a Toll-like receptor (TLR) agonist, a cell-based therapy, a cytokine, a cancer vaccine, and an immune checkpoint modulator of an immune checkpoint molecule.

In one embodiment, the TLR agonist is selected from Coley's toxin and Bacille Calmette-Guérin (BCG).

In one embodiment, the immune checkpoint molecule is selected from CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA.

In one embodiment, the immune checkpoint molecule is a stimulatory immune checkpoint molecule and the immune checkpoint modulator is an agonist of the stimulatory immune checkpoint molecule.

In one embodiment, the immune checkpoint molecule is an inhibitory immune checkpoint molecule and the immune checkpoint modulator is an antagonist of the inhibitory immune checkpoint molecule.

In one embodiment, the immune checkpoint modulator is selected from a small molecule, an inhibitory RNA, an antisense molecule, and an immune checkpoint molecule binding protein.

In one embodiment, the immune checkpoint molecule is PD-1 and the immune checkpoint modulator is a PD-1 inhibitor.

In one embodiment, the PD-1 inhibitor is selected from pembrolizumab, nivolumab, pidilizumab, SHR-1210, MEDI0680R01, BBg-A317, TSR-042, REGN2810 and PF-06801591.

In one embodiment, the immune checkpoint molecule is PD-L1 and the immune checkpoint modulator is a PD-L1 inhibitor.

In one embodiment, the PD-L1 inhibitor is selected from durvalumab, atezolizumab, avelumab, MDX-1105, AMP-224 and LY3300054.

In one embodiment, the immune checkpoint molecule is CTLA-4 and the immune checkpoint modulator is a CTLA-4 inhibitor.

In one embodiment, the CTLA-4 inhibitor is selected from ipilimumab, tremelimumab, JMW-3B3 and AGEN1884.

In embodiments, the agent that induces iron-dependent cellular disassembly is administered before, after or concurrently with administration of the immune checkpoint modulator.

In one embodiment, a response of the cancer to treatment is improved relative to a treatment with the immune checkpoint modulator alone.

In embodiments, the response is improved, e.g., in a population of subjects, by at least 5%, at least 10%, at least 15%, at least 20%, at least 30%, at least 40%, at least 50%, at least 60%, at least 70%, or at least 80% relative to treatment with the immune checkpoint modulator alone.

In one embodiment, the response comprises any one or more of reduction in tumor burden, reduction in tumor size, inhibition of tumor growth, achievement of stable cancer in a subject with a progressive cancer prior to treatment, increased time to progression of the cancer, and increased time of survival.

In one embodiment, the agent that induces iron-dependent cellular disassembly and the immune checkpoint modulator act synergistically.

In one embodiment, the cancer is a cancer responsive to an immune checkpoint therapy.

In embodiments, the cancer is selected from a carcinoma, sarcoma, lymphoma, melanoma, and leukemia.

In various embodiments, the cancer is selected from melanoma, renal cell carcinoma, non-small cell lung cancer, non-squamous cell lung cancer, urothelial carcinoma, Hodgkin's lymphoma, head and neck squamous cell carcinoma, hepatocellular carcinoma, colorectal cancer, gastric adenocarcinoma, gastric esophageal junction adenocarcinoma, and Merkel cell carcinoma.

In a particular embodiment, the cancer is renal cell carcinoma.

In one embodiment, the subject is human.

In one embodiment, the agent that induces iron-dependent cellular disassembly is selected from the group consisting of an inhibitor of antiporter system Xc⁻, an inhibitor of GPX4, and a statin.

In one embodiment, the inhibitor of antiporter system Xc⁻ is erastin or a derivative or analog thereof.

In one embodiment, the analog of erastin is PE or IKE.

In one embodiment, the inhibitor of GPX4 is selected from the group consisting of (1S,3R)-RSL3 or a derivative or analog thereof, ML162, DPI compound 7, DPI compound 10, DPI compound 12, DPI compound 13, DPI compound 17, DPI compound 18, DPI compound 19, FIN56, and FIN02.

In one embodiment, the statin is selected from the group consisting of atorvastatin, fluvastatin, lovastatin, pitavastatin, pravastatin, rosuvastatin, cerivastatin and simvastatin.

In one embodiment, the agent that induces iron-dependent cellular disassembly is selected from the group consisting of sorafenib or a derivative or analog thereof, sulfasalazine, glutamate, BSO, DPI2, cisplatin, cysteinase, silica based nanoparticles, CCI4, ferric ammonium citrate, trigonelline and brusatol.

In one embodiment, the agent that induces iron-dependent cellular disassembly has one or more of the following characteristics:

(a) induces iron-dependent cellular disassembly of a target cell in vitro and subsequent activation of an immune response in a co-cultured cell;

(b) induces iron-dependent cellular disassembly of a target cell in vitro and subsequent activation of co-cultured macrophages, e.g., RAW264.7 macrophages;

(c) induces iron-dependent cellular disassembly of a target cell in vitro and subsequent activation of co-cultured monocytes, e.g., THP-1 monocytes;

(d) induces iron-dependent cellular disassembly of a target cell in vitro and subsequent activation of co-cultured bone marrow-derived dendritic cells (BMDCs);

(e) induces iron-dependent cellular disassembly of a target cell in vitro and subsequent increase in levels or activity of NFkB, IRF and/or STING in a co-cultured cell;

(f) induces iron-dependent cellular disassembly of a target cell in vitro and subsequent increase in levels or activity of a pro-immune cytokine in a co-cultured cell; and

(g) induces iron-dependent cellular disassembly of a target cell in vitro and subsequent activation of co-cultured CD4+ cells, CD8+ cells and/or CD3+ cells;

In embodiments, the agent that induces iron-dependent cellular disassembly is targeted to a cancer cell.

In certain aspects, the disclosure relates to a method of screening for an immunostimulatory agent, the method comprising:
(a) providing a plurality of test agents (e.g., a library of test agents);
(b) evaluating each of the plurality of test agents for the ability to induce iron-dependent cellular disassembly;
(c) selecting as a candidate immunostimulatory agent a test agent that induces iron-dependent cellular disassembly; and
(d) evaluating the candidate immunostimulatory agent for the ability to stimulate an immune response.

In one embodiment, the evaluating step (b) comprises contacting cells or tissue with each of the plurality of test agents.

In one embodiment, the evaluating step (b) comprises administering each of the plurality of test agents to an animal.

In one embodiment, the evaluating step (b) further comprises measuring the level or activity of a marker selected from the group consisting of lipid peroxidation, reactive oxygen species (ROS), isoprostanes, malondialdehyde (MDA), iron, glutathione peroxidase 4 (GPX4), prostaglandin-endoperoxide synthase 2 (PTGS2), cyclooxygenase-2 (COX-2), and glutathione (GSH) in the cells or tissue contacted with the test agent.

In one embodiment, the evaluating step (b) further comprises comparing the level or activity of the marker in the cells or tissue contacted with the test agent to the level or activity of the marker in a control cell or tissue that has not been contacted with the test agent.

In one embodiment, the evaluating step (d) comprises evaluating the test agent that induces iron-dependent cellular disassembly for immunostimulatory activity.

In one embodiment, the evaluating step (d) comprises measuring immune response in an animal.

In one embodiment, an increase in the level or activity of a marker selected from the group consisting of lipid peroxidation, isoprostanes, reactive oxygen species (ROS), iron, PTGS2 and COX-2, or a decrease in the level or activity of a marker selected from the group consisting of GPX4, MDA and GSH indicates that the test agent is an agent that induces iron-dependent cellular disassembly.

In one embodiment, evaluating the candidate immunostimulatory agent comprises culturing an immune cell together with cells contacted with the selected candidate immunostimulatory agent or exposing an immune cell to postcellular signaling factors produced by cells contacted with the selected candidate immunostimulatory agent and measuring the level or activity of NFκB, IRF or STING in the immune cell.

In one embodiment, the immune cell is a THP-1 cell.

In one embodiment, evaluating the candidate immunostimulatory agent comprises culturing T cells together with cells contacted with the selected candidate immunostimulatory agent or exposing T cells to postcellular signaling factors produced by cells contacted with the selected candidate immunostimulatory agent and measuring the activation and proliferation of the T cells.

In certain aspects, the disclosure relates to a method of identifying an immunostimulatory agent, the method comprising:
(a) contacting a cell with an agent that induces iron-dependent cellular disassembly in an amount sufficient to induce iron-dependent cellular disassembly in the cell; (b) isolating one or more postcellular signaling factors produced by the cell after contact with the agent that induces iron-dependent cellular disassembly; and
(c) assaying the one or more postcellular signaling factors for the ability to stimulate immune response.

In one embodiment, the method further comprises selecting a test agent that stimulates immune response.

In one embodiment, the method further comprises detecting a marker of iron-dependent cellular disassembly in the cell.

In one embodiment, the method further comprises:
i) measuring the level of the one or more postcellular signaling factors produced by the cell after contact with the agent that induces iron-dependent cellular disassembly;

ii) comparing the level of the one or more postcellular signaling factors produced by the cell after contact with the agent that induces iron-dependent cellular disassembly to the level of the one or more test agents in a control cell that is not treated with the agent that induces iron-dependent cellular disassembly; and
iii) selecting postcellular signaling factors that exhibit increased levels in the cell contacted with the agent that induces iron-dependent cellular disassembly relative to the control cell to generate the one or more postcellular signaling factors for assaying in step (c).

In one embodiment, the control cell is treated with an agent that induces a cell death that is not iron-dependent cellular disassembly.

In one embodiment, the assaying comprises administering the one or more postcellular signaling factors to an animal and measuring immune response in the animal.

In one embodiment, the assaying comprises treating an immune cell with the one or more postcellular signaling factors and measuring the level or activity of NFκB activity in the immune cell.

In one embodiment, the assaying comprises treating T cells with the one or more postcellular signaling factors and measuring the activation or proliferation of the T cells. In one embodiment, the assaying comprises contacting an immune cell with the one or more postcellular signaling factors and measuring the level or activity of NFκB, IRF or STING in the immune cell.

In one embodiment, the immune cell is a THP-1 cell.

### BRIEF DESCRIPTION OF THE DRAWINGS

FIG. 1A shows HT1080 fibrosarcoma cells treated with various concentrations of erastin. FIG. 1B shows NFkB activity in THP1 monocytes co-cultured with HT1080 cells treated with erastin. Error bars represent standard deviation among three replicates.

FIG. 1C shows HT1080 fibrosarcoma cells treated with DMSO or various concentrations of erastin (ERAS) or the erastin analogs piperazine erastin (PE) or imidazole ketoerastin (IKE). The DMSO control is on the far left. The erastin or erastin analog concentrations increase from left to right and are the same as those shown in FIG. 1A.

FIG. 1D shows NFkB activity in THP1 monocytes co-cultured with HT1080 cells treated with erastin (ERAS) or the erastin analogs piperazine erastin (PE) or imidazole ketoerastin (IKE). The DMSO control is on the far left. The erastin or erastin analog concentrations increase from left to right and are the same as those shown in FIG. 1B. Error bars represent standard deviation among three replicates.

FIG. 2A shows pancreatic cancer cells (PANC1) treated with various concentrations of erastin. FIG. 2B shows NFkB activity in THP1 monocytes co-cultured with PANC1 cells treated with erastin.

FIG. 3A shows renal cell carcinoma cells (Caki-1) treated with various concentrations of erastin. FIG. 3B shows NFkB activity in THP1 monocytes co-cultured with Caki-1 cells treated with erastin.

FIG. 4A shows renal cell carcinoma cells (Caki-1) treated with various concentrations of RSL3. FIG. 4B shows NFkB activity in THP1 monocytes co-cultured with Caki-1 cells treated with RSL3.

FIG. 5A shows Jurkat T cell leukemia cells treated with various concentrations of RSL3. FIG. 5B shows NFkB activity in THP1 monocytes co-cultured with Jurkat cells treated with RSL3.

FIG. 6A shows A20 B-cell leukemia cells treated with various concentrations of RSL3. FIG. 6B shows NFkB activity in THP1 monocytes co-cultured with A20 cells treated with RSL3. FIG. 6C shows IRF activity in THP1 monocytes co-cultured with A20 cells treated with RSL3.

FIG. 7A shows the viability of HT1080 fibrosarcoma cells treated with various concentrations of Erastin alone or in combination with a ferroptosis inhibitor (Ferrostatin-1, Liproxstatin-1 or Trolox).

FIG. 7B shows NFkB activity in THP1 monocytes co-cultured with HT1080 fibrosarcoma cells treated with Erastin alone or in combination with a ferroptosis inhibitor (Ferrostatin-1, Liproxstatin-1 or Trolox).

FIG. 8A shows the viability of HT1080 fibrosarcoma cells treated with various concentrations of Erastin alone or in combination with a ferroptosis inhibitor (Ferrostatin-1, 3-Mercaptoethanol, or Deferoxamine).

FIG. 8B shows NFkB activity in THP1 monocytes co-cultured with HT1080 fibrosarcoma cells treated with Erastin alone or in combination with a ferroptosis inhibitor (Ferrostatin-1, β-Mercaptoethanol, or Deferoxamine).

FIG. 9A shows the viability of HT1080 fibrosarcoma cells treated with various concentrations of Erastin in combination with an siRNA control (siControl) or an siRNA directed to the ACSL4 gene (siACSL4).

FIG. 9B shows the viability of H1080 fibrosarcoma cells treated with DMSO or Erastin in combination with an siRNA control (siControl), an siRNA directed to the ACSL4 gene (siACSL4), or an siRNA directed to the CARS gene (siCARS).

FIG. 9C shows the fold change in NFkB activity in THP1 monocytes co-cultured with HT1080 fibrosarcoma cells treated with DMSO or Erastin in combination with an siRNA control (siControl), an siRNA directed to the ACSL4 gene (siACSL4), or an siRNA directed to the CARS gene (siCARS).

FIG. 10A shows the viability of A20 lymphoma cells treated with DMSO or various concentrations of RSL3 alone or in combination with Ferrostatin-1.

FIG. 10B shows NFkB activity in THP1 monocytes co-cultured with A20 lymphoma cells treated with DMSO or various concentrations of RSL3 alone or in combination with Ferrostatin-1.

FIG. 11A shows the viability of A20 lymphoma cells treated with DMSO or various concentrations of ML162 alone or in combination with Ferrostatin-1.

FIG. 11B shows NFkB activity in THP1 monocytes co-cultured with A20 lymphoma cells treated with DMSO or various concentrations of ML162 alone or in combination with Ferrostatin-1.

FIG. 12A shows the viability of A20 lymphoma cells treated with DMSO or various concentrations of ML210 alone or in combination with Ferrostatin-1.

FIG. 12B shows NFkB activity in THP1 monocytes co-cultured with A20 lymphoma cells treated with DMSO or various concentrations of ML210 alone or in combination with Ferrostatin-1.

FIG. 13A shows the viability of Caki-1 renal carcinoma cells treated with DMSO or various concentrations of RSL3 alone or in combination with Ferrostatin-1.

FIG. 13B shows NFkB activity in THP1 monocytes co-cultured with Caki-1 renal carcinoma cells treated with DMSO or various concentrations of RSL3 alone or in combination with Ferrostatin-1.

FIG. 14A shows the viability of Caki-1 renal carcinoma cells treated with DMSO or various concentrations of ML162 alone or in combination with Ferrostatin-1.

FIG. 14B shows NFkB activity in THP1 monocytes co-cultured with Caki-1 renal carcinoma cells treated with DMSO or various concentrations of ML162 alone or in combination with Ferrostatin-1.

### DETAILED DESCRIPTION

The present disclosure relates to methods of increasing immune activity in a cell, tissue or subject comprising administering to the cell, tissue or subject an agent that induces iron-dependent cellular disassembly. Applicants have surprisingly shown that induction of iron-dependent cellular disassembly (e.g. ferroptosis) increases immune response as evidenced by increases in NFKB and IRF activity in immune cells. Accordingly, administration of an agent that induces iron-dependent cellular disassembly may be used to treat disorders that would benefit from increased immune activity, such as cancer or an infection.

### I. Definitions

The terms “administer”, “administering” or “administration” include any method of delivery of a pharmaceutical composition or agent into a subject's system or to a particular region in or on a subject.

As used herein, “administering in combination”, “co-administration” or “combination therapy” is understood as administration of two or more active agents using separate formulations or a single pharmaceutical formulation, or consecutive administration in any order such that, there is a time period while both (or all) active agents overlap in exerting their biological activities. It is contemplated herein that one active agent (e.g., an agent that induces iron-dependent cellular disassembly) can improve the activity of a second agent, for example, can sensitize target cells, e.g., cancer cells, to the activities of the second agent. “Administering in combination” does not require that the agents are administered at the same time, at the same frequency, or by the same route of administration. As used herein, “administering in combination”, “co-administration” or “combination therapy” includes administration of an agent that induces iron-dependent cellular disassembly with one or more additional anti-cancer agents, e.g., immune checkpoint modulators. Examples of immune checkpoint modulators are provided herein.

“Ferroptosis”, as used herein, refers to a process of regulated cell death that is iron dependent and involves the production of reactive oxygen species.

“Cellular disassembly” refers to a dynamic process that reorders and disseminates the material within a cell and may ultimately result in cell death. The cellular disassembly process includes the production and release from the cell of postcellular signaling factors.

As used herein, the terms “increasing” (or “activating”) and “decreasing” refer to modulating resulting in, respectively, greater or lesser amounts, function or activity of a parameter relative to a reference. For example, subsequent to administration of a preparation described herein, a parameter (e.g., activation of NFkB, activation of macrophages, size or growth of a tumor) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration. Generally, the metric is measured subsequent to administration at a time that the administration has had the recited effect, e.g., at least one day, one week, one month, 3 months, 6 months, after a treatment regimen has begun. Similarly, pre-clinical parameters (such as activation of NFkB of cells in vitro, and/or reduction in tumor burden of a test mammal, by a preparation described herein) may be increased by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration.

As used herein, “an anti-neoplastic agent” refers to a drug used for the treatment of cancer. Anti-neoplastic agents include chemotherapeutic agents (e.g., alkylating agents, antimetabolites, anti-tumor antibiotics, topoisomerase inhibitors, mitotic inhibitors corticosteroids, and enzymes), biologic anti-cancer agents, and immune checkpoint modulators.

A “cancer treatment regimen” or “anti-neoplastic regimen” is a clinically accepted dosing protocol for the treatment of cancer that includes administration of one or more anti-neoplastic agents to a subject in specific amounts on a specific schedule.

As used herein, an “immune checkpoint” or “immune checkpoint molecule” is a molecule in the immune system that modulates a signal. An immune checkpoint molecule can be a stimulatory checkpoint molecule, i.e., increase a signal, or inhibitory checkpoint molecule, i.e., decrease a signal. A “stimulatory checkpoint molecule” as used herein is a molecule in the immune system that increases a signal or is co-stimulatory. An “inhibitory checkpoint molecule”, as used herein is a molecule in the immune system that decreases a signal or is co-inhibitory.

As used herein, an “immune checkpoint modulator” is an agent capable of altering the activity of an immune checkpoint in a subject. In certain embodiments, an immune checkpoint modulator alters the function of one or more immune checkpoint molecules including, but not limited to, CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. The immune checkpoint modulator may be an agonist or an antagonist of the immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In other embodiments, the immune checkpoint modulator is a small molecule. In a particular embodiment, the immune checkpoint modulator is an anti-PD1, anti-PD-L1, or anti-CTLA-4 binding protein, e.g., antibody or antibody fragment.

An “immunotherapeutic” as used herein refers to a pharmaceutically acceptable compound, composition or therapy that induces or enhances an immune response. Immunotherapeutics include, but are not limited to, immune checkpoint modulators, Toll-like receptor (TLR) agonists, cell-based therapies, cytokines and cancer vaccines.

As used herein, “oncological disorder” or “cancer” or “neoplasm” refer to all types of cancer or neoplasm found in humans, including, but not limited to: leukemias, lymphomas, melanomas, carcinomas and sarcomas. As used herein, the terms “oncological disorder”, “cancer,” and “neoplasm,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells.

Specific criteria for the staging of cancer are dependent on the specific cancer type based on tumor size, histological characteristics, tumor markers, and other criteria known by those of skill in the art. Generally, cancer stages can be described as follows: (i) Stage 0, Carcinoma in situ; (ii) Stage I, Stage II, and Stage III, wherein higher numbers indicate more extensive disease, including larger tumor size and/or spread of the cancer beyond the organ in which it first developed to nearby lymph nodes and/or tissues or organs adjacent to the location of the primary tumor; and (iii) Stage IV, wherein the cancer has spread to distant tissues or organs.

“Postcellular signaling factors” are molecules and cell fragments produced by a cell undergoing cellular disassembly (e.g., iron-dependent cellular disassembly) that are ultimately released from the cell and influence the biological activity of other cells. Postcellular signaling factors can include proteins, peptides, carbohydrates, lipids, nucleic acids, small molecules, and cell fragments (e.g. vesicles and cell membrane fragments).

A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

A “subject” to be treated by the methods of the invention can mean either a human or non-human animal, preferably a mammal, more preferably a human. In certain embodiments, a subject has a detectable or diagnosed cancer prior to initiation of treatments using the methods of the invention. In certain embodiments, a subject has a detectable or diagnosed infection, e.g., chronic infection, prior to initiation of treatments using the methods of the invention.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated. A therapeutically effective amount need not be curative. A therapeutically effective amount need not prevent a disease or condition from ever occurring. Instead a therapeutically effective amount is an amount that will at least delay or reduce the onset, severity, or progression of a disease or condition.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy).

### II. Iron-dependent Cellular Disassembly

Cellular disassembly is a dynamic process that re-orders and disseminates the material within a cell, and which results in the production and release of postcellular signaling factors, or “effectors”, that can have a profound effect on the biological activity of other cells. Cellular disassembly occurs during the process of regulated cell death and is controlled by multiple molecular mechanisms. Different types of cellular disassembly result in the production of different postcellular signaling factors and thereby mediate different biological effects. For example, Applicants have surprisingly shown that induction of an iron-dependent cellular disassembly can increase immune response as evidenced by increases in NFKB and IRF activity in immune cells.

In some embodiments, the iron-dependent cellular disassembly is ferroptosis. Ferroptosis is a process of regulated cell death involving the production of iron-dependent reactive oxygen species (ROS). In some embodiments, ferroptosis involves the iron-dependent accumulation of lipid hydroperoxides to lethal levels. The sensitivity to ferroptosis is tightly linked to numerous biological processes, including amino acid, iron, and polyunsaturated fatty acid metabolism, and the biosynthesis of glutathione, phospholipids, NADPH, and Coenzyme Q10. Ferroptosis involves metabolic dysfunction that results in the production of both cytosolic and lipid ROS, independent of mitochondria but dependent on NADPH oxidases in some cell contexts (Dixon et al., 2012, Cell 149(5):1060-72).

Agents that Induce Iron-Dependent Cellular Disassembly

Provided herein are agents that induce iron-dependent cellular disassembly. Such agents are capable of inducing the process of iron-dependent cellular disassembly when present in sufficient amount and for a sufficient period of time. In certain embodiments, the agent that induces iron-dependent cellular disassembly induces the process of iron-dependent cellular disassembly in a cell such that post-cellular signaling factors, such as immunostimulatory post-cellular signaling factors, are produced by the cell, but does not result in cell death. In other embodiments, the agent that induces iron-dependent cellular disassembly induces the process of iron-dependent cellular disassembly in a portion of a cell population, e.g., 10%, 20%, 30%, 40%, 50%, 60%, 70%, 80%, 90%, 95% or more cells of the population, such that post-cellular signaling factors, e.g., immunostimulatory post-cellular signaling factors, are produced by the portion of cells in the cell population. Cell death may occur in all or only a fraction of the portion of cells in the cell population.

A broad range of agents that induce iron-dependent cellular disassembly, e.g., ferroptosis, are known in the art, and are useful in the various methods provided by the present invention. For example, two oncogenic RAS Selective Lethal (RSL) small molecules named eradicator of Ras and ST (erastin) and Ras Selective Lethal 3 (RSL3) were initially identified as small molecules that are selectively lethal to cells expressing oncogenic mutant RAS proteins, a family of small GTPases that are commonly mutated in cancer. (See Cao et al., 2016, Cell Mol Life Sci 73: 2195-2209, incorporated in its entirety herein.) Specifically, in engineered human fibroblast cell lines, the small molecule erastin was found to induce preferential lethality in cells overexpressing oncogenic HRAS (see Dolma et al., 2003, Cancer Cell. 3:285-296, incorporated in its entirety herein). Erastin functionally inhibits the cystine-glutamate antiporter system Xc−. System Xc− is a heterodimeric cell surface amino acid antiporter composed of the twelve-pass transmembrane transporter protein SLC7A11 (xCT) linked by a disulfide bridge to the single-pass transmembrane regulatory protein SLC3A2 (4F2hc, CD98hc). Antiporter system Xc-imports extracellular cystine, the oxidized form of cysteine, in exchange for intracellular glutamate. (See Cao et al., 2016, Cell Mol Life Sci 73: 2195-2209, incorporated in its entirety herein.) Cells treated with erastin are deprived of cysteine and are unable to synthesize the antioxidant glutathione. Depletion of glutathione eventually leads to excessive lipid peroxidation and increased ROS which triggers iron-dependent cellular disassembly. Erastin-induced ferroptotic cell death is distinct from apoptosis, necrosis, and autophagy, based on morphological, biochemical, and genetic criteria. (See Yang et al., 2014, Cell 156: 317-331, incorporated in its entirety herein.)

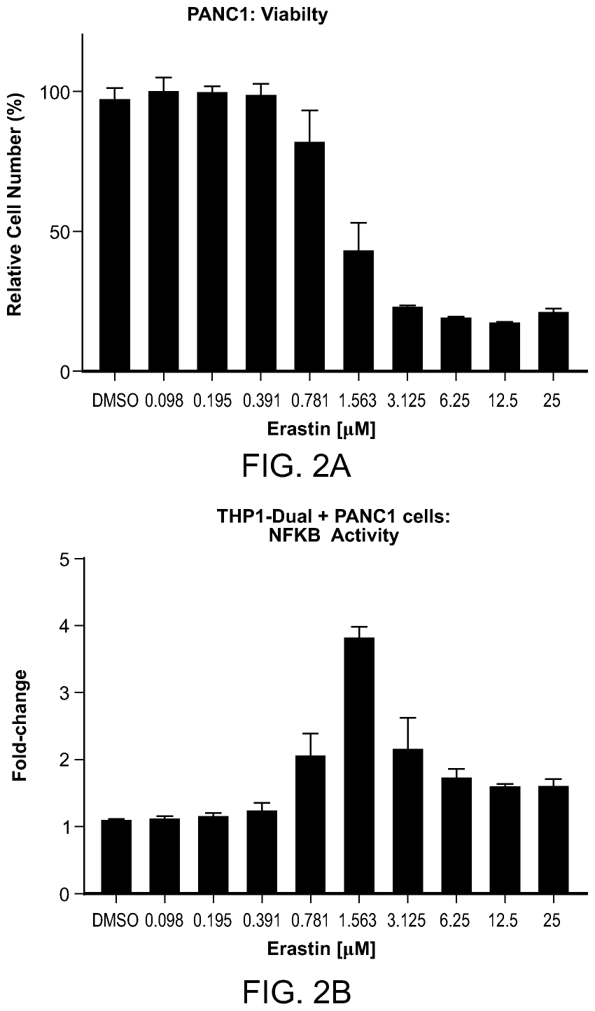

In some embodiments, an agent that induces iron-dependent cellular disassembly, e.g., ferroptosis, and is useful in the methods provided herein is an inhibitor of antiporter system Xc−. Inhibitors of antiporter system Xc− include antiporter system Xc− binding proteins (e.g., antibodies or antibody fragments), nucleic acid inhibitors (e.g., antisense oligonucleotides, or siRNAs), and small molecules that specifically inhibit antiporter system Xc−. For example, in some embodiments, the inhibitor of antiporter system Xc− is a binding protein, e.g., antibody or antibody fragment, that specifically inhibits SLC7A11 or SLC3A2. In some embodiments, the inhibitor of antiporter system Xc− is a nucleic acid inhibitor that specifically inhibits SLC7A11 or SLC3A2. In some embodiments, the inhibitor of antiporter system Xc− is small molecule that specifically inhibits SLC7A11 or SLC3A2. Antibody and nucleic acid inhibitors are well known in the art and are described in detail herein. Small molecule inhibitors of antiporter system Xc− include, but are not limited to, erastin, sulfasalazine, sorafenib, and analogs or derivatives thereof. (See Cao et al., 2016, Cell Mol Life Sci 73: 2195-2209, e.g., FIG. 2, incorporated in its entirety herein).

In a particular embodiment, an agent that induces iron-dependent cellular disassembly, e.g., ferroptosis, is erastin or an analog or derivative thereof. Analogs of erastin include, but are not limited to, the compounds listed in Table 1 below. Each of the references listed in Table 1 is incorporated by reference herein in its entirety.

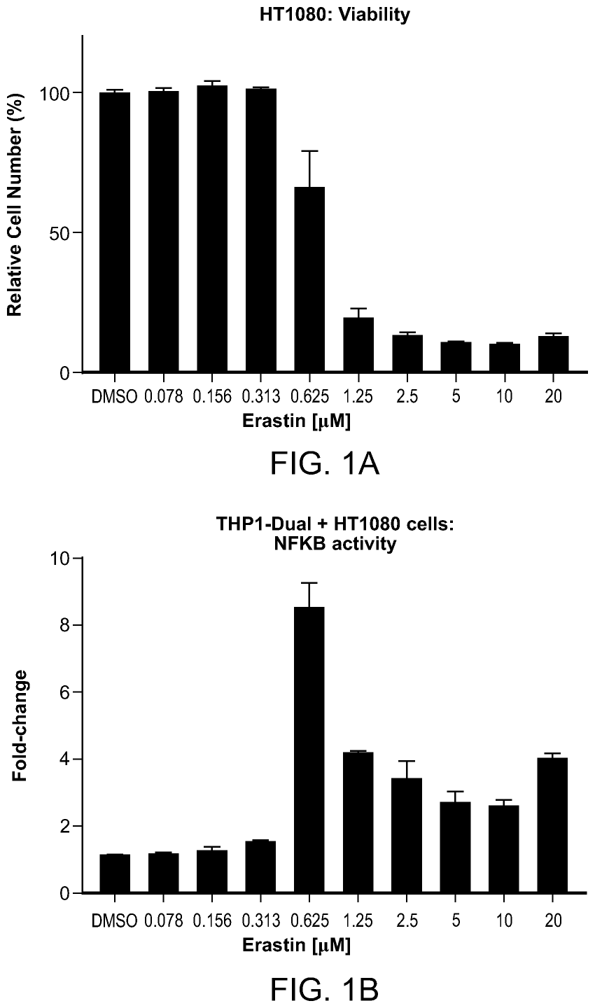

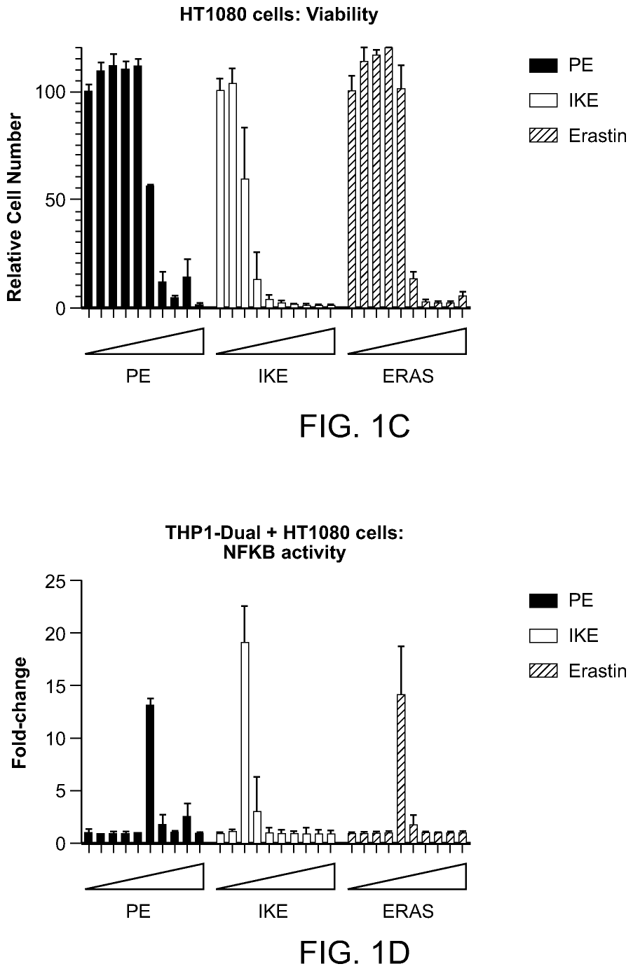

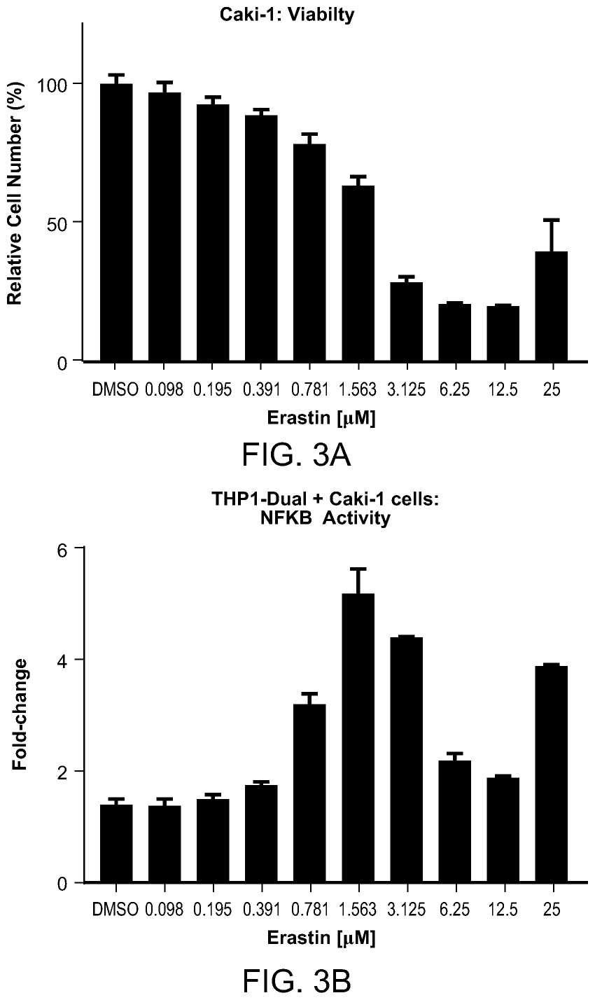

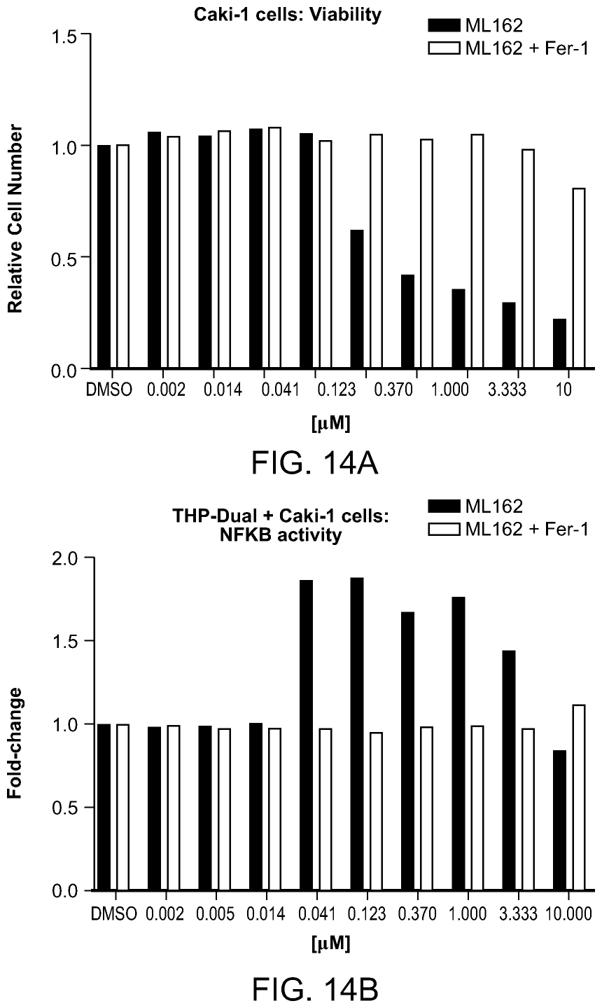

TABLE 1
Erastin Analogs

| Compound | Reference |
|---|---|
| PE | Yang et al., 2014, Cell 156: 317-331; FIG. 1C |
| AE | Yang et al., 2014, Cell 156: 317-331; FIG. 1C |
| IKE | Larraufie, et al., 2015, Bioorg MedChemLetters 25(21): |
| | 4787-4792; FIG. 3 |
| PKE | Larraufie, et al., 2015, Bioorg MedChemLetters 25(21): |
| | 4787-4792; FIG. 3 |
| 35MEW28, 21 | Dixon et al. 2014, eLife February 25; 4. doi: |
| | 10.7554/eLife.05608; FIG. 3B/C |
| KE | US 2016/0332974, pages 10-12, paragraph 18 |
| FKE | US 2016/0332974, pages 10-12, paragraph 18 |
| TFKE | US 2016/0332974, pages 10-12, paragraph 18 |
| APKE | US 2016/0332974, pages 10-12, paragraph 18 |
| MKE | US 2016/0332974, pages 10-12, paragraph 18 |
| PMB-PKE | US 2016/0332974, pages 10-12, paragraph 18 |
| MPKE | US 2016/0332974, pages 10-12, paragraph 18 |
| erastin B1 | U.S. Pat. No. 8,535,897, FIG. 14 |
| erastin B2 | U.S. Pat. No. 8,535,897, FIG. 14 |
| erastin A2 | U.S. Pat. No. 8,535,897, FIG. 14 |
| erastin A3 | U.S. Pat. No. 8,535,897, FIG. 14 |

As used herein, unless indicated otherwise, the term “erastin”, includes any pharmaceutically acceptable form of erastin, including, but not limited to, N-oxides, crystalline form, hydrates, salts, esters, and prodrugs thereof. As used herein, the term “erastin derivatives or erastin analogs” refers to compounds having similar structure and function to erastin. In some embodiments, erastin derivatives/erastin analogs include those of the following formula:

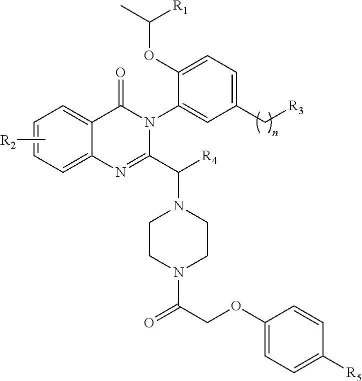

or pharmaceutically acceptable salts or esters thereof, wherein R₁ is selected from the group consisting of H, C₁₋₄ alkyl, C₁₋₄ alkoxy, hydroxy, and halogen;

R₂ is selected from the group consisting of H, halo, and C₁₋₄ alkyl; R₃ is selected from the group consisting of H, C₁₋₄ alkyl, C₁₋₄ alkoxy, 5-7 membered heterocycloalkyl, and 5-6 membered heteroaryl; R₄ is selected from the group consisting of H and C₁₋₄ alkyl;

R₅ is halo;

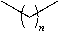

is optionally substituted with ═O; and

n is an integer from 0-4.

In one embodiment, the erastin derivative or analog is a compound represented by Structural Formula (I):

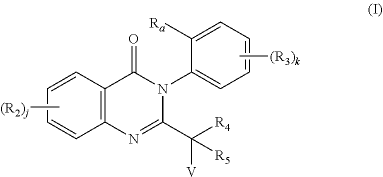

or a pharmaceutically acceptable salt thereof, wherein:

Ra is a halogen, substituted or unsubstituted alkyl, substituted or unsubstituted alkenyl, substituted or unsubstituted alkynyl, substituted or unsubstituted aryl-O—, substituted or unsubstituted alkyl-O—, substituted or unsubstituted alkenyl-O— or substituted or unsubstituted alkynyl-O—, where alkyl, alkenyl and alkynyl are optionally interrupted by NR, O or S(O)ₙ;

each R₂ is independently selected from the group consisting of halogen, substituted or unsubstituted alkyl, substituted or unsubstituted aryl, substituted or unsubstituted non-aromatic heterocyclic, —CN, —COOR′, —CON(R)₂, —NRC(O)R, —SO₂N(R)₂, —N(R)₂, —NO2, —OH and —OR′;

each R₃ is independently selected from the group consisting of halogen, substituted or unsubstituted alkyl, substituted or unsubstituted aryl, substituted or unsubstituted non-aromatic heterocyclic, —(CO)R, —CN, —COOR′, —CON(R)₂, —NRC(O)R, —SO₂N(R)₂, —N(R)₂, —NO2, —OH and —OR′;

R₄ and R₅ are independently selected from the group consisting of —H, substituted or unsubstituted alkyl, substituted or unsubstituted alkenyl, substituted or unsubstituted alkynyl, substituted or unsubstituted non-aromatic heterocyclic and substituted or unsubstituted aryl, where alkyl, alkenyl and alkynyl are optionally interrupted by NR, O or S(O)ₙ; or R₄ and R₅ taken together form a carbocyclic or heterocyclic group;

V is —NH-L-A-Q or

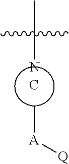

wherein Ring C is a substituted or unsubstituted heterocyclic aromatic or non-aromatic ring;

A is NR or O; or A is a covalent bond;

L is a substituted or unsubstituted hydrocarbyl group optionally interrupted by one or more heteroatoms selected from N, O and S;

Q is selected from the group consisting of —R, —C(O)R′, —C(O)N(R)₂, —C(O)OR′, and —S(O)₂R′;

each R is independently —H, alkyl, alkenyl, alkynyl, aryl, or non-aromatic heterocyclic, wherein said alkyl, alkenyl, alkynyl, aryl, or non-aromatic heterocyclic groups are substituted or unsubstituted;

each R′ is independently an alkyl, alkenyl, alkynyl group, non-aromatic heterocyclic or aryl group, wherein said alkyl, alkenyl, alkynyl, non-aromatic heterocyclic or aryl groups are substituted or unsubstituted;

j is an integer from 0 to 4;

k is an integer from 0 to 4, provided that at least one of j and k is an integer from 1 to 4;

and each n is independently 0, 1 or 2.

In another embodiment, the erastin derivative is a compound represented by Structural Formula (I) as disclosed in the above embodiment; wherein V is

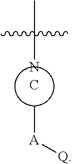

Suitable examples of V encompassed by the above structure include:

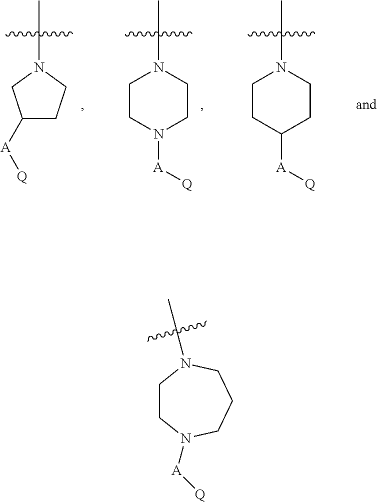

and wherein all other variables are as disclosed in the above mentioned embodiment.

In one embodiment, the erastin derivative or analog is a compound represented by Structural Formula (II):

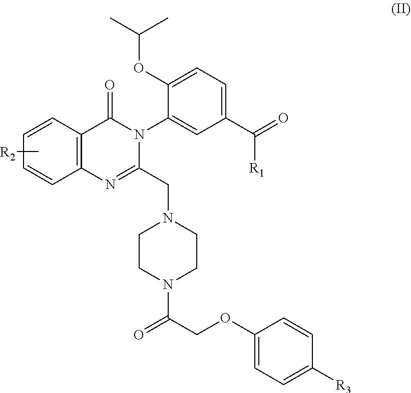

wherein R₁ is selected from the group consisting of H, C₁₋₆ alkyl, and CF₃, wherein each C₁₋₆ alkyl may be optionally substituted with an atom or a group selected from the group consisting of a halogen atom, a saturated or unsaturated C₃₋₆-heterocycle and an amine, each heterocycle optionally substituted with an atom or group selected from the group consisting of C₁₋₄ aliphatic, which C₁₋₄ aliphatic may be optionally substituted with an C₁₋₄ alkyl-aryl-O—C₁₋₄alkyl;

R₂ is selected from the group consisting of H, halo, and C₁₋₆ aliphatic; and

R₃ is a halo atom;

or an N-oxide, crystalline form, hydrate, or pharmaceutically acceptable salt thereof.

In another embodiment, the erastin derivative or analog is a compound represented by Structural Formula (III):

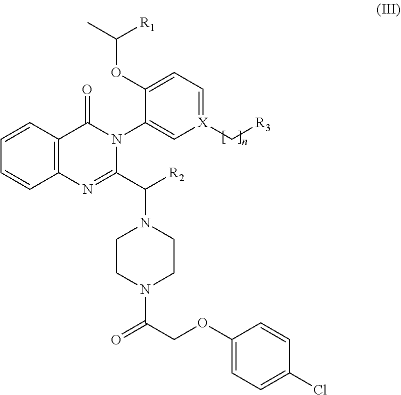

wherein

R₁ is selected from the group consisting of H, C₁₋₄ alkyl, C₁₋₄ alkoxy, hydroxy, and halogen;

R₂ is selected from the group consisting of H, C₁₋₄ alkyl, C₁₋₄ alkoxy, C₃₋₈ cycloalkyl, C₃₋₈ heterocycloalkyl, aryl, heteroaryl, and C₁₋₄ aralkyl;

R₃ is absent, or is selected from the group consisting of C₁₋₄ alkyl, C₁₋₄ alkoxy, carbonyl, C₃₋₈ cycloalkyl, and C₃₋₈ heterocycloalkyl;

X is selected from the group consisting of C, N, and O; and

n is an integer from 0-6,

with the proviso that when X is C, n=0, and R₃ is absent, R₁ cannot be H when R₂ is CH₃;

or an N-oxide, crystalline form, hydrate, or pharmaceutically acceptable salt thereof.

In a particular embodiment, the erastin derivative is a compound represented by Structural Formula (IV):

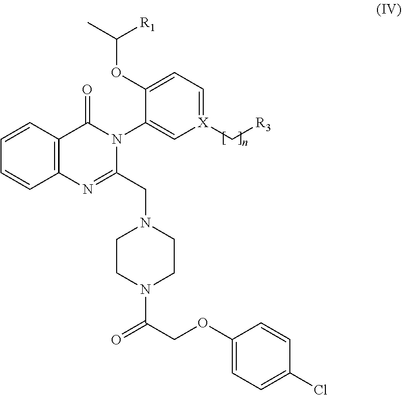

or an N-oxide, crystalline form, hydrate, or pharmaceutically acceptable salt thereof,

wherein the definitions for all the variables are as defined in the above embodiment disclosing compound of formula (III).

In another embodiment, the erastin derivative or analog is a compound represented by Structural Formula (V):

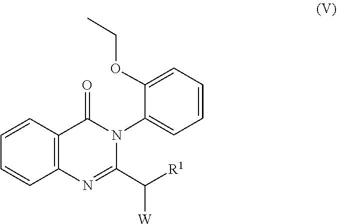

or a pharmaceutically acceptable salt thereof,

wherein R¹ is selected from H, —Z-Q-Z, —C₁₋₈ alkyl-N(R²)(R⁴), —C₁₋₈ alkyl-OR³, 3- to 8-membered carbocyclic or heterocyclic, aryl, heteroaryl, and C₁₋₄aralkyl;

R² and R⁴ are each independently for each occurrence selected from H, C₁₋₄ alkyl, C₁₋₄ aralkyl, aryl, heteroaryl, acyl, alkylsulfonyl, and arylsulfonyl, provided that when both R² and R⁴ are on the same N atom and not both H, they are different, and that when both R² and R⁴ are on the same N and either R² or R⁴ is acyl, alkylsulfonyl, or arylsulfonyl, then the other is selected from H, C₁₋₈ alkyl, C₁₋₄ aralkyl, aryl, and heteroaryl;

R³ is selected from H, C₁₋₄ alkyl, C₁₋₄ aralkyl, aryl, and heteroaryl;

W is selected from

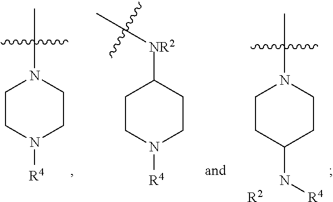

Q is selected from O and NR²; and

Z is independently for each occurrence selected from C₁₋₆ alkyl, C₂₋₆ alkenyl, and C₂₋₆ alkynyl. When Z is an alkenyl or alkynyl group, the double or triple bond or bonds are preferably not at the terminus of the group (thereby excluding, for example, enol ethers, alkynol ethers, enamines and/or ynamines).

In a particular embodiment, the compound is represented by Structural Formula (V) of the above disclosed embodiment; wherein

R² and R⁴ are each independently for each occurrence selected from H, C₁₋₄ alkyl, C₁₋₄ aralkyl, aryl, heteroaryl, acyl, alkylsulfonyl, and arylsulfonyl, provided that when both R² and R⁴ are on the same N atom and not both H, they are different;

R³ is selected from H, C₁₋₄ alkyl, aryl, and heteroaryl;

Z is independently for each occurrence selected from C₁₋₆ alkyl, C₂₋₆ alkenyl, and C₂₋₆ alkynyl;

wherein each heterocyclic group is a 3 to 10 membered non-aromatic ring including one to four heteroatoms selected from nitrogen, oxygen, and sulfur;

wherein each aryl is phenyl;

wherein each heteroaryl is a 5 to 7 membered aromatic ring including one to four heteroatoms selected from nitrogen, oxygen, and sulfur; and

wherein each heterocyclic, aryl, and heteroaryl group is optionally substituted by one or more moieties selected from the group consisting of halogen, hydroxyl, carboxyl, alkoxycarbonyl, formyl, acyl, thioester, thioacetate, thioformate, alkoxyl, phosphoryl, phosphate, phosphonate, phosphinate, amino, amido, amidino, imino, cyano, nitro, azido, sulfhydryl, alkylthio, sulfate, sulfonate, sulfamoyl, and sulfonamido.

The term “substituted” refers to moieties having substituents replacing a hydrogen on one or more carbons of the backbone. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

In a particular embodiment, the inhibitor of antiporter system Xc⁻ is

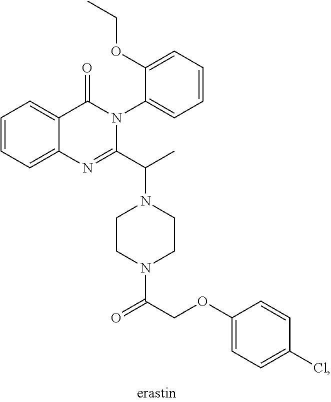 [SMILES: CCOc1ccccc1-n1c(C(C)N2CCN(C(=O)COc3ccc(Cl)cc3)CC2)nc2ccccc2c1=O]

or a pharmaceutically acceptable salt thereof.

In a particular embodiment, the inhibitor of antiporter system Xc⁻ is

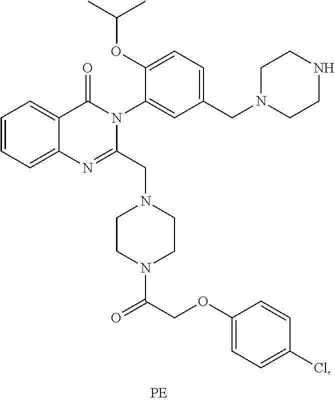 [SMILES: CC(C)Oc1ccc(CN2CCNCC2)cc1-n1c(CN2CCN(C(=O)COc3ccc(Cl)cc3)CC2)nc2ccccc2c1=O]

or a pharmaceutically acceptable salt thereof.

In a particular embodiment, the inhibitor of antiporter system Xc⁻ is

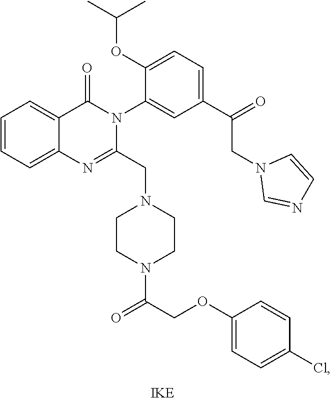 [SMILES: CC(C)Oc1ccc(C(=O)Cn2ccnc2)cc1-n1c(CN2CCN(C(=O)COc3ccc(Cl)cc3)CC2)nc2ccccc2c1=O]

or a pharmaceutically acceptable salt thereof.

Additional erastin derivatives or analogs are described, for example in WO 2015/109009, U.S. Pat. Nos. 9,695,133, 8,535,897, WO 2015/051149, US 2008/0299076, US2007/0161644, WO 2008/013987, U.S. Pat. Nos. 8,575,143, 8,518,959, WO 2007/076085, Bioorganic & Medicinal Chemistry Letters (2015), 25(21), 4787-4792, eLife (2014), 3, Letters in Organic Chemistry (2015), 12(6), 385-393, Pharmacia Lettre (2012), 4(5), 1344-1351, PLoS Pathogens (2014), 10(6), Bioorganic & Medicinal Chemistry Letters (2011), 21(18), 5239-5243, Indian Journal of Chemistry, Section B: Organic Chemistry Including Medicinal Chemistry (2010), 49B(7), 923-928, Synthetic Communications (2009), 39(18), 3217-3231, Indian Journal of Chemistry, Section B: Organic Chemistry Including Medicinal Chemistry (1994), 33B(3), 260-5, Journal of Heterocyclic Chemistry (1983), 20(5), 1339-49, Chemical & Pharmaceutical Bulletin (1979), 27(11), 2675-87, Journal of Medicinal Chemistry (1977), 20(3), 379-86, Indian Journal of Chemistry (1971), 9(3), 201-6, and Journal of Medicinal Chemistry (1968), 11(2), 392-5, each of which is incorporated by reference herein in its entirety.

In some embodiments, an agent that induces iron-dependent cellular disassembly (e.g., ferroptosis) and is useful in the methods provided herein is an inhibitor of glutathione peroxidase 4 (GPX4). GPX4 is a phospholipid hydroperoxidase that catalyzes the reduction of hydrogen peroxide and organic peroxides, thereby protecting cells against membrane lipid peroxidation, or oxidative stress. Thus, GPX4 contributes to a cell's ability to survive in oxidative environments. Inhibition of GPX4 can induce cell death by ferroptosis (see, Yang, W. S., et al. Regulation of ferroptotic cancer cell death by GPX4. Cell 156, 317-331 (2014)). Inhibitors of GPX4 include GPX4-binding proteins (e.g., antibodies or antibody fragments), nucleic acid inhibitors (e.g., antisense oligonucleotides or siRNAs), and small molecules that specifically inhibit GPX4. Small molecule inhibitors of GPX4 include, but are not limited to, the compounds listed in Table 2 below. Each of the references listed in Table 2 is incorporated by reference herein in its entirety.

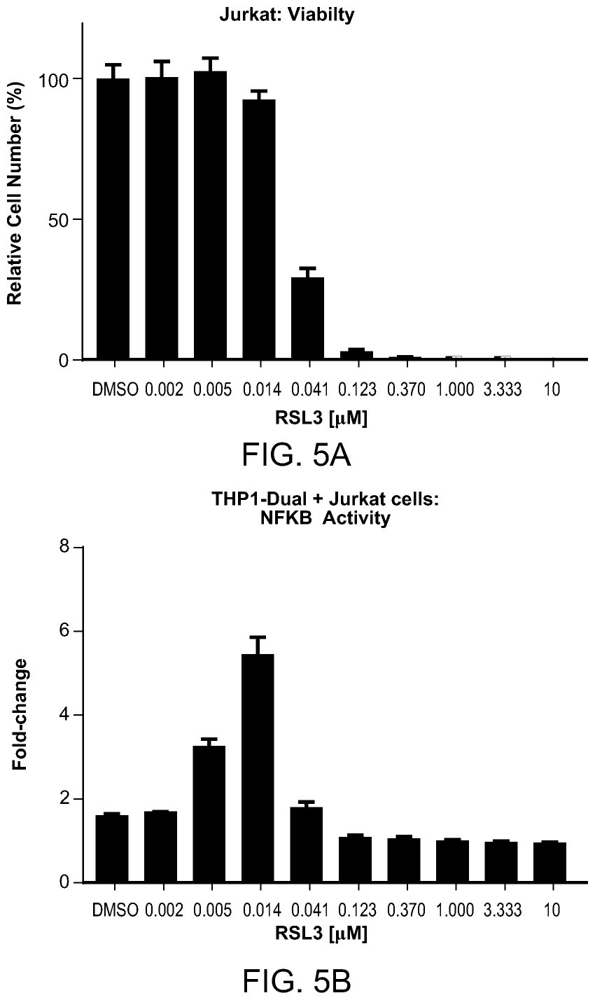

TABLE 2
GPX4 inhibitors

| Compound | Reference |
|---|---|
| DPI7 (ML162), | Yang et al., 2014, Cell 156: 317-331; FIGS. 5 |
| DPI19, DPI17, | and S5 |
| DPI13, DPI12 | |
| DPI10 (ML210) | Cao et al., 2016, Cell Mol Life Sci 73: 2195-2209; |
| | FIG. 2 |
| RSL3, or a derivative | Cao et al., 2016, Cell Mol Life Sci 73: 2195-2209; |
| or analog thereof | FIG. 2 |
| altretamine | Cao et al., 2016, Cell Mol Life Sci 73: 2195-2209; |
| | FIG. 2 |
| FIN56 | Shimada et al., 2016, Nat. Chem Biol. 12(7): |
| | 497-503 |
| FINO2 | Gaschler et al., 2018, Nature Chemical Biology |
| | 14: 507-515 |

In a particular embodiment, the GPX4 inhibitor is

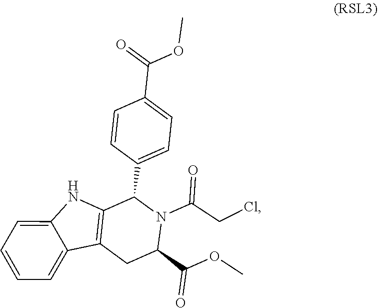 [SMILES: COC(=O)c1ccc([C@H]2c3[nH]c4ccccc4c3C[C@H](C(=O)OC)N2C(=O)CCl)cc1]

or a pharmaceutically acceptable salt thereof.

RSL3 is a known inhibitor of GPX4. In knockdown studies, RSL3 selectively mediated the death of RAS-expressing cells and was identified as increasing lipid ROS accumulation. See U.S. Pat. No. 8,546,421.

In some embodiments, the inhibitor of GPX4 is a diastereoisomer of RSL3.

In a particular embodiment, the diastereoisomer of RSL3 is

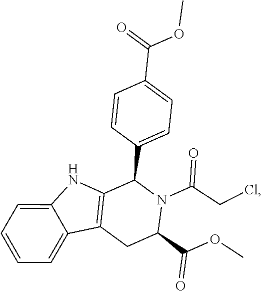 [SMILES: COC(=O)c1ccc([C@@H]2c3[nH]c4ccccc4c3C[C@H](C(=O)OC)N2C(=O)CCl)cc1]

or a pharmaceutically acceptable salt thereof.

In a particular embodiment, the diastereoisomer of RSL3 is

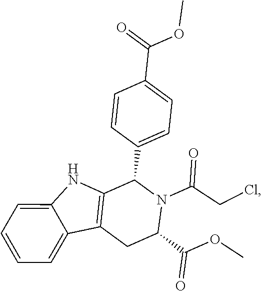 [SMILES: COC(=O)c1ccc([C@H]2c3[nH]c4ccccc4c3C[C@@H](C(=O)OC)N2C(=O)CCl)cc1]

or a pharmaceutically acceptable salt thereof.

In a particular embodiment, the diastereoisomer of RSL3 is

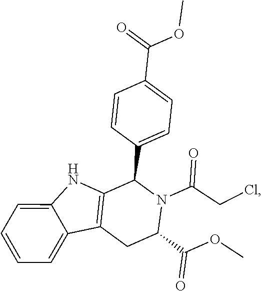 [SMILES: COC(=O)c1ccc([C@@H]2c3[nH]c4ccccc4c3C[C@@H](C(=O)OC)N2C(=O)CCl)cc1]

or a pharmaceutically acceptable salt thereof.

In some embodiments, the inhibitor of GPX4 is a pharmaceutically acceptable form of RSL3, including, but not limited to, N-oxides, crystalline form, hydrates, salts, esters, and prodrugs thereof.

In some embodiments, the inhibitor of GPX4 is RSL3 or a derivative or analog thereof. Derivatives and analogs of RSL3 are known in the art and are described, for example, in WO2008/103470, WO2017/120445, WO2018118711, U.S. Pat. No. 8,546,421, and CN108409737, each of which is incorporated by reference herein in its entirety.

In some embodiments, the RSL3 derivative or analog is a compound represented by Structural Formula (I):

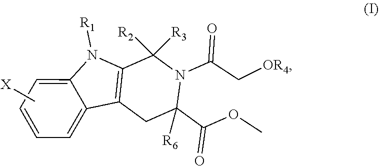

or an enantiomer, optical isomer, diastereomer, N-oxide, crystalline form, hydrate, or pharmaceutically acceptable salt thereof, wherein

R₁, R₂, R₃, and R₆ are independently selected from H, C₁₋₈alkyl, C₁₋₈alkoxy, C₁₋₈aralkyl, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkoxy, aralkyl, carbocyclic, heterocyclic, aryl, heteroaryl, acyl, alkylsulfonyl, and arylsulfonyl is optionally substituted with at least one substituent;

R₄ and R₅ are independently selected from H₁ C₁₋₈alkyl, C₁₋₈alkoxy, 3- to 8-membered carbocyclic, 3- to 8-membered heterocyclic, 3- to 8-membered aryl, or 3-to 8-membered heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether is optionally substituted with at least one substituent;

R⁷ is selected from H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;

R⁸ is selected from H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; and

X is 0-4 substituents on the ring to which it is attached.

In one embodiment, the RSL3 derivative or analog is a compound represented by Structural Formula (II):

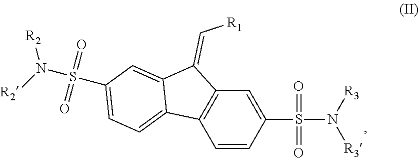

or an N-oxide, crystalline form, hydrate, or pharmaceutically acceptable salt thereof; wherein:

R₁ is selected from the group consisting of H, OH, and —(OCH₂CH₂)ₓPH;

X is an integer from 1 to 6; and

R₂, R₂′, R₃, and R₃′ independently are selected from the group consisting of H, C₃₋₈cycloalkyl, and combinations thereof, or R₂ and R₂′ may be joined together to form a pyridinyl or pyranyl and R₃ and R₃′ may be joined together to form a pyridinyl or pyranyl.

In one embodiment, the RSL3 derivative or analog is a compound represented by Structural Formula (III):

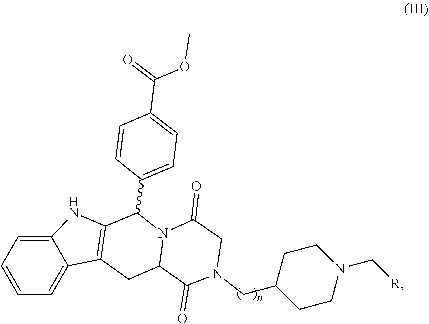

or a stereoisomer thereof, or a pharmaceutically acceptable salt thereof; wherein: n is 2, 3 or 4; and R is a substituted or unsubstituted C₁-C₆ alkyl group, a substituted or unsubstituted C₃-C₁₀ cycloalkyl group, a substituted or unsubstituted C₂-C₈ heterocycloalkyl group, a substituted or unsubstituted C₆-C₁₀ aromatic ring group, or a substituted or unsubstituted C₃-C₈ heteroaryl ring group; wherein the substitution means that one or more hydrogen atoms in each group are substituted by the following groups selected from the group consisting of: halogen, cyano, nitro, hydroxy, C₁-C₆ alkyl, halogenated C₁-C₆ alkyl, C₁-C₆ alkoxy, halogenated C₁-C₆ alkoxy, COOH (carboxy), COOC₁-C₆ alkyl, OCOC₁-C₆ alkyl.

In some embodiments, the GPX4 inhibitor is

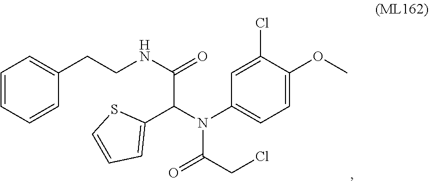 [SMILES: COc1ccc(N(C(=O)CCl)C(C(=O)NCCc2ccccc2)c2cccs2)cc1Cl]

or a pharmaceutically acceptable salt thereof.

ML162 has been identified as a direct inhibitor of GPX4 that induces ferroptosis (see, Dixon et al., 2015, ACS Chem. Bio. 10, 1604-1609).

In some embodiments, the GPX4 inhibitor is a pharmaceutically acceptable form of ML162, including, but not limited to, N-oxides, crystalline form, hydrates, salts, esters, and prodrugs thereof.

In some embodiments, the inhibitor of GPX4 is ML162 or a derivative or analog thereof.

In some embodiments, the GPX4 inhibitor is

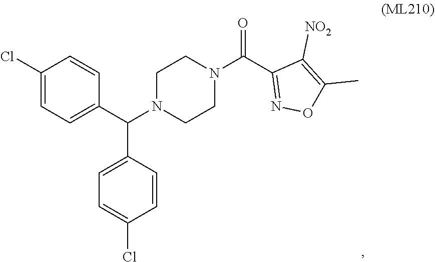 [SMILES: Cc1onc(C(=O)N2CCN(C(c3ccc(Cl)cc3)c3ccc(Cl)cc3)CC2)c1[N+](=O)[O-]]

or a pharmaceutically acceptable salt thereof.

In some embodiments, the GPX4 inhibitor is a pharmaceutically acceptable form of ML210, including, but not limited to, N-oxides, crystalline form, hydrates, salts, esters, and prodrugs thereof.

In some embodiments, the inhibitor of GPX4 is ML210 or a derivative or analog thereof.

In some embodiments, the inhibitor of GPX4 is FIN56 or a derivative or analog thereof.

In one embodiment, FIN56 or a derivative or analog thereof is represented by formula:

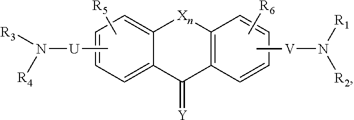

or a pharmaceutically acceptable salt, ester, amide, stereoisomer, geometric isomer, solvate or prodrug thereof, wherein

n=0-2, and wherein when n=1, X is selected from CH₂, 0, NR_{A}, CO, and C═NOR_{A} and wherein when n=2, X═CH₂,

Y is O, S, NOR_{A}, or NR_{A},
wherein R_{A} is selected from H, alkyl, heteroalkyl, alkenyl, alkynyl, cycloalkyl, —C(═O)R_{B}, —C(═O)OR_{B}, —C(═O)NR_{B}R_{C}, —C(═NR_{B})R_{C}, —NR_{B}Rc, heterocycloalkyl, aryl or polyaromatic, heteroaryl, arylalkyl and alkylaryl,
wherein each of said R_{B} and Rc is independently H, alkyl, or heteroalkyl, U and V are each independently selected from C═O, and O═S═O and wherein when U is C═O, V is not C═O,

R₁, R₂, R₃, and R₄ are each independently selected from H, alkyl, heteroalkyl, cycloalkyl, arylcycloalkyl, alkenyl, alkynyl, aryl, heteroaryl, heterocycloalkyl, and each of said NR₁R₂ and NR₃R₄ can independently combine to form a heterocycloalkyl,

R₅ and R₆ are each independently selected from H, OH, SH, alkoxy, thioalkoxy, alkyl, halogen, CN, CF₃, NO₂, COOR_{D}, CONR_{D}R_{E}, NR_{D}R_{E}, NR_{D}COR_{E}, NR_{D}SO₂R_{E}, and NR_{F}CONR_{D}R_{E};
wherein RD, RE and RF are independently H, alkyl, heteroalkyl, aryl, arylalkyl, heteroaryl, heteroarylalkyl, cycloalkyl, or heterocycloalkyl;

provided that if X is O, Y is O and U and V are both O=S═O, then NR₁R₂ and NR₃R₄ are not identical then R₁ and R₃ are each independently selected from H and lower alkyl, and wherein R₂ and R₄ are each independently selected from lower alkoxy(loweralkyl), di(lower)alkylamino(lower)alkyl, halobenzyl, morpholino(lower)alkyl, or NR₁R₂ and NR₃R₄ are independently piperidino, morpholino, piperazino, N-phenylpiperazino, ethylamino, or substituted glycine,

and wherein if X is (CH₂)2, Y is O or NOH, and U and V are each O=S═O then none of R₁, R₂, R₃, and R₄ is methyl,

and wherein if n=O, Y is O or NOH, and U and V are each O=S═O, then NR₁R₂ and NR₃R₄ are not identical and R₁, R₂, R₃, and R₄ are each independently selected from C₁-C₅ alkyl, C₁O alkyl, Ciβ alkyl, C₁₇ alkyl, phenyl, benzyl, naphthalenyl, piperizino, pyridinyl, pyrazolyl, benzimidazolyl, triazolyl; or NR₁R₂ and NR₃R₄ are independently piperidino, morpholino, or piperazino,

and wherein if X is CO, Y is O, and U and V are each O=S═O then NR₁R₂ and NR₃R₄ are not identical, and wherein R₁, R₂, R₃, and R₄ are each independently selected from methyl, ethyl, hydroxy-d-Cralkyl, SH, RO, COOH, SO, NH₂, and phenyl or wherein one or both of non-identical NR₁R₂ and NR₃R₄ is unsubstituted piperidino, N-methylpiperazino or N-methylhomopiperazino,

and wherein when X is C═O or C═NOH, Y is O or NOH, and U and V are each O=S═O and one of R₁ or R₂ and one of R₃ or R₄ is phenyl then the other of R₁ or R₂ and R₃ or R₄ is not H or alkyl.

In one embodiment, the FIN56 derivative or analog thereof is represented by the following formula:

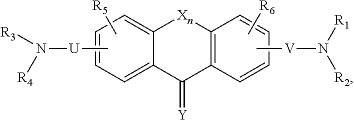

wherein n=1-2 and wherein when n=1, X is selected from CH₂, O, CO, and C═NOR_{A}; and wherein when n=2, X═CH₂,

Y is O, S, NOR_{A}, or NR_{A},

wherein U and V are each O=S═O,

wherein R_{A} is selected from H, alkyl, heteroalkyl, alkenyl, alkynyl, cycloalkyl, —C(═O)RB, —C(═O)OR_{B}, —C(═O)NR_{B}R_{C}, —C(═NR_{B})R_{C}, —NR_{B}R_{C}, heterocycloalkyl, aryl or polyaromatic, heteroaryl, arylalkyl and alkylaryl,

wherein each of said R_{B} and R_{C} is independently H, alkyl, or heteroalkyl,

R₁, R₂, R₃, and R₄ are each independently selected from H, alkyl, heteroalkyl, cycloalkyl, arylcycloalkyl, alkenyl, alkynyl, aryl, heteroaryl, heterocycloalkyl, and each of said NR₁R₂ and NR₃R₄ can independently combine to form a heterocycloalkyl,

R₅ and R₆ are each independently selected from H, OH, SH, alkoxy, thioalkoxy, alkyl, halogen, CN, CF₃, NO₂, COOR_{D}, CONR_{D}R_{E}, NR_{D}R_{E}, NR_{D}COR_{E}, NR_{D}SO₂R_{E}, and NR_{F}CONR_{D}R_{E};
wherein R_{D}, R_{E} and R_{F} are independently H, alkyl, heteroalkyl, aryl, arylalkyl, heteroaryl, heteroarylalkyl, cycloalkyl, or heterocycloalkyl; provided that is X is O, Y is O and U and V are both O=S═O, then NR₁R₂ and NR₃R₄ are not identical then R₁ and R₃ are each independently selected from H and lower alkyl, and wherein R₂ and R₄ are each independently selected from lower alkoxy(loweralkyl), di(lower)alkylamino(lower)alkyl, halobenzyl, morpholino(lower)alkyl, or NR₁R₂ and NR₃R₄ are independently piperidino, morpholino, piperazino, N-phenylpiperazino, ethylamino, or substituted glycine,

and wherein if X is (CH₂)₂, and Y is O or NOH, then none of R₁, R₂, R₃, and R₄ is methyl,

and wherein if X is CO and Y is O, then NR₁R₂ and NR₃R₄ are not identical, and wherein R₁, R₂, R₃, and R₄ are each independently selected from methyl, ethyl, hydroxy-C₁-C₃-alkyl, SH, RO, COOH, SO, NH₂, and phenyl or wherein one or both of non-identical NR₁R₂ and NR₃R₄ is unsubstituted piperidino, N-methylpiperazino or N-methylhomopiperazino, wherein said unsubstituted piperidine, N-methylpiperazino or N-methylhomopiperazino NR₁R₂ and NR₃R₄ moieties are not identical,

and wherein when X is C═O or C═NOH, Y is O or NOH, and one of R₁ or R₂ and one of R₃ or R₄ is phenyl then the other of R1 or R₂ and R₃ or R₄ is not H or alkyl,

or a pharmaceutically acceptable salt, ester, amide, stereoisomer or geometric isomer thereof.

In one embodiment, the FIN56 derivative or analog thereof is represented by the following formula:

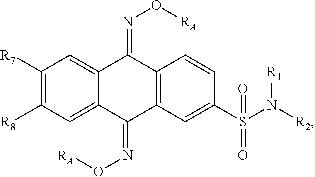

wherein R_{A} is hydrogen, R₇ and R₈ are independently selected from H and SO₂NR₃R₄, wherein one of R₇ and R₈ is hydrogen and wherein NR₁R₂ and NR₃R₄ are independently 6- to 15-membered heterocycloalkyl containing one nitrogen in the ring, or a pharmaceutically acceptable salt, ester, amide, stereoisomer or geometric isomer thereof.

Additional FIN56 derivatives or analogs are described, for example in WO 2008/140792, WO 2010/082912, WO 2017/058716, U.S. Pat. No. 6,693,136, Nature Chemical Biology (2016), 12(7), 497-503 doi: 10.1038/nchembio.2079, ACS Chemical Biology (2015), 10(7), 1604-1609 doi: 10.1021/acschembio.5b00245, Dissertation Abstracts International, (2015) Vol. 76, No. 8B(E). Order No.: AAI3688566. ProQuest Dissertations & Theses. 120 pages, each of which is incorporated by reference herein in its entirety.

In some embodiments, an agent that induces iron-dependent cellular disassembly (e.g., ferroptosis) and is useful in the methods provided herein is a statin. In one embodiment, the statin is selected from the group consisting of atorvastatin, fluvastatin, lovastatin, pitavastatin, pravastatin, rosuvastatin,cerivastatin and simvastatin.

In one embodiment, the agent that induces iron-dependent cellular disassembly (e.g., ferroptosis) and is useful in the methods provided herein is selected from the group consisting of glutamate, BSO, DPI2 (See Yang et al., 2014, Cell 156: 317-331; FIG. 5 and S5, incorporated in its entirety herein), cisplatin, cysteinase, silica based nanoparticles, CCI4, ferric ammonium citrate, trigonelline and brusatol.

Additional agents that induce iron-dependent cellular disassembly are known in the art and are described, for example in U.S. Pat. Nos. 8,518,959; 8,535,897; 8,546,421; 9,580,398; 9,695,133; US2010/0081654; US2015/0079035; US2015/0175558; US2016/0229836; US2016/0297748; US2016/0332974; Cell. 2012 May 25; 149(5):1060-72. doi: 10.1016/j.cell.2012.03.042;
Cell. 2014 January 16; 156(1-2):317-331. doi: 10.1016/j.cell.2013.12.010;
J Am Chem Soc. 2014 March 26; 136(12):4551-6. doi: 10.1021/ja411006a;
Elife. 2014 May 20; 3:e02523. doi: 10.7554/eLife.02523;
Proc Natl Acad Sci USA. 2014 Nov. 25; 111(47):16836-41. doi: 10.1073/pnas.1415518111;
Nat Cell Biol. 2014 December; 16(12):1180-91. doi: 10.1038/ncb3064;
ACS Chem Biol. 2015 Jul. 17; 10(7):1604-9. doi: 10.1021/acschembio.5b00245;
Bioorg Med Chem Lett. 2015 Nov. 1; 25(21):4787-92. doi: 10.1016/j.bmcl.2015.07.018;
Nat Rev Drug Discov. 2016 May; 15(5):348-66. doi: 10.1038/nrd.2015.6;
Cell Chem Biol. 2016 Feb. 18; 23(2):225-235. doi:10.1016/j.chembiol.2015.11.016;
Nat Chem Biol. 2016 July; 12(7):497-503. doi: 10.1038/nchembio.2079;
Proc Natl Acad Sci USA. 2016 Aug. 23; 113(34):E4966-75. doi: 10.1073/pnas.1603244113;
ACS Cent Sci. 2016 Sep. 28; 2(9):653-659;
Nat Chem Biol. 2017 January; 13(1):81-90. doi: 10.1038/nchembio.2238;
Biochem Biophys Res Commun. 2017 Jan. 15; 482(3):419-425. doi: 10.1016/j.bbrc.2016.10.086; Nat Chem Biol. 2018 May; 14(5):507-515. doi: 10.1038/s41589-018-0031-6;
Cancer Lett. 2018 Apr. 24. pii: 50304-3835(18)30288-X. doi: 10.1016/j.canlet.2018.04.021;
J Med Chem. 2018 Apr. 24. doi: 10.1021/acs.jmedchem.8b00315;
Eur J Med Chem. 2018 May 10; 151:434-449. doi: 10.1016/j.ejmech.2018.04.005;
Neuropharmacology. 2018 Mar. 15; 135:242-252. doi: 10.1016/j.neuropharm.2018.03.015;
J Am Chem Soc. 2018 Mar. 14; 140(10):3798-3808. doi: 10.1021/jacs.8b00998;
Biochem Biophys Res Commun. 2018 Feb. 26; 497(1):233-240. doi: 10.1016/j.bbrc.2018.02.061;
Org Biomol Chem. 2018 Feb. 28; 16(9):1465-1479. doi: 10.1039/c7ob03086j;
Arch Toxicol. 2018 April; 92(4):1507-1524. doi: 10.1007/s00204-018-2170-7;
Int J Oncol. 2018 March; 52(3):1011-1022. doi: 10.3892/ijo.2018.4259;
Cancer Lett. 2018 Apr. 28; 420:210-227. doi: 10.1016/j.canlet.2018.01.061.Feb 1;
Free Radic Biol Med. 2018 March; 117:45-57. doi: 10.1016/j.freeradbiomed.2018.01.019;
Sci Rep. 2018 Jan. 12; 8(1):574. doi: 10.1038/s41598-017-18935-1;
Cancer Lett. 2018 Mar. 1; 416:124-137. doi: 10.1016/j.canlet.2017.12.025;
ChemMedChem. 2018 Jan. 22; 13(2):164-177. doi: 10.1002/cmdc.201700629;
Redox Biol. 2018 April; 14:535-548. doi: 10.1016/j.redox.2017.11.001;
Arch Toxicol. 2018 February; 92(2):759-775. doi: 10.1007/s00204-017-2066-y;
Nat Chem. 2017 October; 9(10):1025-1033. doi: 10.1038/nchem.2778;
Free Radic Biol Med. 2017 November; 112:597-607. doi: 10.1016/j.freeradbiomed.2017.09.002;
ACS Chem Biol. 2017 Oct. 20; 12(10):2538-2545. doi: 10.1021/acschembio.7b00730. 20;
PLoS One. 2017 Aug. 21; 12(8):e0182921. doi: 10.1371/journal.pone.0182921. eCollection
2017; Biochem Biophys Res Commun. 2017 Sep. 30; 491(4):919-925. doi: 10.1016/j.bbrc.2017.07.136;
Biochem Pharmacol. 2017 Sep. 15; 140:41-52. doi: 10.1016/j.bcp.2017.06.112. 23;
Cancer Res Treat. 2018 April; 50(2):445-460. doi: 10.4143/crt.2016.572;
Cell Death Discov. 2017 Feb. 27; 3:17013. doi: 10.1038/cddiscovery.2017.13. eCollection 2017; Nat Nanotechnol. 2016 November; 11(11):977-985. doi: 10.1038/nnano.2016.164;
Biochem Biophys Res Commun. 2016 Nov. 25; 480(4):602-607. doi: 10.1016/j.bbrc.2016.10.099; Oncotarget. 2016 Nov. 15; 7(46):74630-74647. doi: 10.18632/oncotarget.11858;
Cancer Lett. 2016 Oct. 10; 381(1):165-75. doi: 10.1016/j.canlet.2016.07.033. 29;
Cell Death Dis. 2016 Jul. 21; 7:e2307. doi:10.1038/cddis.2016.208;
Oncol Rep. 2016 August; 36(2):968-76. doi: 10.3892/or.2016.4867. 31;
Biochem Biophys Res Commun. 2016 May 13; 473(4):775-780. doi: 10.1016/j.bbrc.2016.03.052; Mol Carcinog. 2017 January; 56(1):75-93. doi: 10.1002/mc.22474;
ACS Chem Biol. 2016 May 20; 11(5):1305-12. doi: 10.1021/acschembio.5b00900;
J Med Chem. 2016 Mar. 10; 59(5):2041-53. doi: 10.1021/acs.jmedchem.5b01641;
Phytomedicine. 2015 Oct. 15; 22(11):1045-54. doi: 10.1016/j.phymed.2015.08.002;
Biol Pharm Bull. 2015; 38(8):1234-9. doi: 10.1248/bpb.b15-00048;
Oncoscience. 2015 May 2; 2(5):517-32. eCollection 2015;
Pathol Oncol Res. 2015 September; 21(4):1115-21. doi: 10.1007/s12253-015-9946-3;
Anticancer Res. 2014 November; 34(11):6417-22; and
Int J Cancer. 2013 Oct. 1; 133(7):1732-42. doi: 10.1002/ijc.28159;
each of which is incorporated by reference herein in its entirety.

In one embodiment, an agent that induces iron-dependent cellular disassembly (e.g., ferroptosis) and is useful in the compositions and methods provided herein induces one or more desirable immune effects in a co-cultured cell, such as an immune cell. For example, in embodiments, the agent that induces iron-dependent cellular disassembly has one or more of the following characteristics:
(a) induces iron-dependent cellular disassembly of a target cell in vitro and activation of an immune response in a co-cultured cell;
(b) induces iron-dependent cellular disassembly of a target cell in vitro and activation of co-cultured macrophages, e.g., RAW264.7 macrophages;
(c) induces iron-dependent cellular disassembly of a target cell in vitro and activation of co-cultured monocytes, e.g., THP-1 monocytes;
(d) induces iron-dependent cellular disassembly of a target cell in vitro and activation of co-cultured bone marrow-derived dendritic cells (BMDCs);
(e) induces iron-dependent cellular disassembly of a target cell in vitro and increases levels or activity of NFkB, IRF and/or STING in a co-cultured cell;
(f) induces iron-dependent cellular disassembly of a target cell in vitro and increases levels or activity of a pro-immune cytokine in a co-cultured cell;
(g) induces iron-dependent cellular disassembly of a target cell in vitro and activation of co-cultured CD4+ cells, CD8+ cells and/or CD3+ cells; and
(h) induces iron-dependent cellular disassembly of a target cell in vitro and increases levels or activity of T cells.
   Numerous methods for determining an agent which, in addition to inducing iron-dependent cellular disassembly of a target cell, induces said immune effects in a co-cultured cell are known in the art and are provided and described in detail herein.

In certain aspects of the invention, it can be desirable to target or direct the delivery of the agent that induces iron-dependent cellular disassembly to a particular target cell, such as a cancer cell. Accordingly, in some embodiments, the agent that induces iron-dependent cellular disassembly is targeted to a cancer cell. Methods of targeting therapeutic agents to cancer cells are known in the art and are described, for example, in US2017/0151345, which is incorporated by reference herein in its entirety. For example, the agent that induces iron-dependent cellular disassembly may be targeted to a cancer cell by combining it, for example in a complex or as a conjugate, with a molecule that specifically binds to a cancer cell marker. As used herein, the term “cancer cell marker” refers to a polypeptide that is present on the surface of a cancer cell. For example, a cancer cell marker may be a cancer cell receptor, e.g., a polypeptide that binds specifically to a molecule in the extracellular environment. A cancer cell marker (e.g., receptor) can be a polypeptide displayed exclusively on cancer cells, a polypeptide displayed at a higher level on cancer cells than normal cells of the same or different tissue types, or a polypeptide displayed on both cancerous and normal cell types. In some embodiments, a cancer cell marker (e.g., receptor) can be a polypeptide that, in cancer cells, has altered (e.g. higher or lower than normal) expression and/or activity. In some embodiments, a cancer cell marker (e.g., receptor) can be a polypeptide that is implicated in the disease process of cancer. In some embodiments, a cancer cell marker (e.g., receptor) can be a polypeptide that is involved in the control of cell death and/or apoptosis. Non-limiting examples of cancer cell markers include, but are not limited to, EGFR, ER, PR, HER2, PDGFR, VEGFR, MET, c-MET, ALK, CD117, RET, DR4, DRS, and FasR. In some embodiments, the molecule that specifically binds to the cancer cell marker (e.g., receptor) is an antibody or cancer cell marker-binding fragment thereof. In some embodiments, the cancer cell marker is a receptor and the molecule that specifically binds to the cancer cell receptor is a ligand or a ligand mimetic of the receptor.

Accordingly, in some embodiments, it is envisaged that a composition of the invention comprises a complex or conjugate comprising the agent that induces iron-dependent cellular disassembly and a molecule that specifically binds to a cancer cell marker (e.g., receptor). In certain embodiments, the complex or conjugate comprises a pharmaceutically acceptable dendrimer, for example, a PAMAM dendrimer. In certain embodiments, the complex comprises a liposome. In certain embodiments, the complex comprises a microparticle or a nanoparticle.

### III. Methods of Increasing Immune Activity

The agents that induce iron-dependent cellular disassembly (e.g., ferroptosis) described herein may be used to increase immune activity in a cell, tissue or in a subject, for example, a subject who would benefit from increased immune activity. For example, in some aspects, the disclosure relates to a method of increasing immune activity in a cell, tissue or subject, the method comprising administering to the cell, tissue or subject an agent that induces iron-dependent cellular disassembly in an amount sufficient to increase the immune activity relative to a cell, tissue or subject that is not treated with the agent that induces iron-dependent cellular disassembly. In some aspects, the disclosure relates to a method of increasing immune activity in a tissue or subject, the method comprising administering to the tissue or subject an agent that induces iron-dependent cellular disassembly in an amount sufficient to increase the immune activity relative to a tissue or subject that is not treated with the agent that induces iron-dependent cellular disassembly.

In one embodiment, the subject is in need of an increased immune activity.

Administration of the agent that induces iron-dependent cellular disassembly results in the production of postcellular signaling factors that regulate immune activity. Immune activity may be regulated by interaction of the postcellular signaling factors with a broad range of immune cells, including mast cells, Natural Killer (NK) cells, basophils, neutrophils, monocytes, macrophages, dendritic cells, eosinophils, and lymphocytes (e.g. B-lymphocytes (B-cells)), and T-lymphocytes (T-cells)).

Mast cells are a type of granulocyte containing granules rich in histamine and heparin, an anti-coagulant. When activated, a mast cell releases inflammatory compounds from the granules into the local microenvironment. Mast cells play a role in allergy, anaphylaxis, wound healing, angiogenesis, immune tolerance, defense against pathogens, and blood-brain barrier function.

Natural Killer (NK) cells are cytotoxic lymphocytes that lyse certain tumor and virus infected cells without any prior stimulation or immunization. NK cells are also potent producers of various cytokines, e.g. IFN-gamma (IFNγ), TNF-alpha (TNFα), GM-CSF and IL-3. Therefore, NK cells are also believed to function as regulatory cells in the immune system, influencing other cells and responses. In humans, NK cells are broadly defined as CD56+CD3-lymphocytes. The cytotoxic activity of NK cells is tightly controlled by a balance between the activating and inhibitory signals from receptors on the cell surface. A main group of receptors that inhibits NK cell activation are the inhibitory killer immunoglobulin-like receptors (KIRs). Upon recognition of self MHC class I molecules on the target cells, these receptors deliver an inhibitory signal that stops the activating signaling cascade, keeping cells with normal MHC class I expression from NK cell lysis. Activating receptors include the natural cytotoxicity receptors (NCR) and NKG2D that push the balance towards cytolytic action through engagement with different ligands on the target cell surface. Thus, NK cell recognition of target cells is tightly regulated by processes involving the integration of signals delivered from multiple activating and inhibitory receptors.

Monocytes are bone marrow-derived mononuclear phagocyte cells that circulate in the blood for few hours/days before being recruited into tissues. See Wacleche et al., 2018, Viruses (10)2: 65. The expression of various chemokine receptors and cell adhesion molecules at their surface allows them to exit the bone marrow into the blood and to be subsequently recruited from the blood into tissues. Monocytes belong to the innate arm of the immune system providing responses against viral, bacterial, fungal or parasitic infections. Their functions include the killing of pathogens via phagocytosis, the production of reactive oxygen species (ROS), nitric oxide (NO), myeloperoxidase and inflammatory cytokines. Under specific conditions, monocytes can stimulate or inhibit T-cell responses during cancer as well as infectious and autoimmune diseases. They are also involved in tissue repair and neovascularization.

Macrophages engulf and digest substances such as cellular debris, foreign substances, microbes and cancer cells in a process called phagocytosis. Besides phagocytosis, macrophages play a critical role in nonspecific defense (innate immunity) and also help initiate specific defense mechanisms (adaptive immunity) by recruiting other immune cells such as lymphocytes. For example, macrophages are important as antigen presenters to T cells. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages.

Dendritic cells (DCs) play a critical role in stimulating immune responses against pathogens and maintaining immune homeostasis to harmless antigens. DCs represent a heterogeneous group of specialized antigen-sensing and antigen-presenting cells (APCs) that are essential for the induction and regulation of immune responses. In the peripheral blood, human DCs are characterized as cells lacking the T-cell (CD3, CD4, CD8), the B-cell (CD19, CD20) and the monocyte markers (CD14, CD16) but highly expressing HLA-DR and other DC lineage markers (e.g., CD1a, CD1c). See Murphy et al., Janeway's Immunobiology. 8th ed. Garland Science; New York, N.Y., USA: 2012. 868p.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g. to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Lymphocytes include B-lymphocytes (B-cells), which are precursors of antibody-secreting cells, and T-lymphocytes (T-cells).

B-Lymphocytes (B-cells)

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B-cell activation requires that the antigen express multiple copies of the epitope complementary to the binding site of the cell surface receptors, because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4⁺ T-cells. The CD4⁺ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4⁺ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

T-Lymphocytes (T-cells)

T-lymphocytes derived from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. While antibody and B-cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8⁺ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class I MHC protein. CD4⁺ T cells recognize only peptide/class II complexes while CD8⁺ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors. This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4th Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4⁺ T cells tend to differentiate into cells that principally secrete the cytokines IL-4, IL-5, IL-6, and IL-10 (T_{H}2 cells) or into cells that mainly produce IL-2, IFN-γ, and lymphotoxin (T_{H}1 cells). The T_{H}2 cells are very effective in helping B-cells develop into antibody-producing cells, whereas the T_{H}1 cells are effective inducers of cellular immune responses, involving enhancement of microbicidal activity of monocytes and macrophages, and consequent increased efficiency in lysing microorganisms in intracellular vesicular compartments. Although CD4⁺ T cells with the phenotype of T_{H}2 cells (i.e., IL-4, IL-5, IL-6 and IL-10) are efficient helper cells, T_{H}1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. T_{H1} cells are effective in enhancing the microbicidal action, because they produce IFN-γ. In contrast, two of the major cytokines produced by T_{H2} cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Schwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4⁺ T (Treg) cells (Reviewed in Kronenberg, M. et al., “Regulation of immunity by self-reactive T cells”, Nature, Vol. 435: 598-604 (2005)). CD4⁺ Tregs that constitutively express the IL-2 receptor alpha (IL-2Rα) chain (CD4⁺CD25⁺) are a naturally occurring T cell subset that are anergic and suppressive (Taams, L. S. et al., “Human anergic/suppressive CD4⁺CD25⁺ T cells: a highly differentiated and apoptosis-prone population”, Eur. J. Immunol. Vol. 31: 1122-1131 (2001)). Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development (Battaglia, M. et al., “Rapamycin promotes expansion of functional CD4⁺CD25⁺Foxp3⁺ regulator T cells of both healthy subjects and type 1 diabetic patients”, J. Immunol., Vol. 177: 8338-8347, (2006)). Accordingly, in some embodiments, an increase in immune response may be associated with a lack of activation or proliferation of regulatory T cells.

Cytotoxic T Lymphocytes

CD8⁺ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

T-Memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269rv1, (2015)).

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rv1, (2015)).

The agent that induces iron-dependent cellular disassembly may increase immune activity in a tissue or subject by inducing production of postcellular signaling factors that increase the level or activity of immune cells described herein, for example, macrophages, monocytes, dendritic cells, and CD4+, CD8+ or CD3+ cells (e.g. CD4+, CD8+ or CD3+ T cells). For example, in one embodiment, the agent that induces iron-dependent cellular disassembly is administered in an amount sufficient to increase in the tissue or subject one or more of: the level or activity of macrophages, the level or activity of monocytes, the level or activity of dendritic cells, the level or activity of T cells, and the level or activity of CD4+, CD8+ or CD3+ cells (e.g. CD4+, CD8+ or CD3+ T cells).

The agent that induces iron-dependent cellular disassembly may also increase immune activity in a cell, tissue or subject by inducing production of postcellular signaling factors that increase the level or activity of a pro-immune cytokine. For example, in some embodiments, the agent that induces iron-dependent cellular disassembly is administered in an amount sufficient to increase in a cell, tissue or subject the level or activity of a pro-immune cytokine. In one embodiment, the pro-immune cytokine is selected from IFN-α, IL-1, IL-12, IL-18, IL-2, IL-15, IL-4, IL-6, TNF-α, IL-17 and GMCSF.

The agent that induces iron-dependent cellular disassembly may also increase immune activity in a cell, tissue or subject by inducing production of postcellular signaling factors that increase the level or activity of positive regulators of the immune response such as nuclear factor kappa-light-chain-enhancer of activated B cells (NFkB), interferon regulatory factor (IRF), and stimulator of interferon genes (STING). For example, in some embodiments, the agent that induces iron-dependent cellular disassembly is administered in an amount sufficient to increase in a cell, tissue or subject the level or activity of NFkB, IRF and/or STING.

In some embodiments, the disclosure relates to a method of increasing immune activity of an immune cell, comprising: (i) contacting a target cell with an agent that induces iron-dependent cellular disassembly and (ii) exposing the immune cell to the target cell that has been contacted with the agent or postcellular signaling factors produced by the target cell that has been contacted with the agent, in an amount sufficient to increase immune activity of the immune cell relative to an immune cell in the absence of contacting the target cell with the agent that induces iron-dependent cellular disassembly.

In some embodiments, the disclosure relates to a method of increasing the level or activity of NFkB in an immune cell, comprising: (i) contacting a target cell with an agent that induces iron-dependent cellular disassembly and (ii) exposing the immune cell to the target cell that has been contacted with the agent or postcellular signaling factors produced by the target cell that has been contacted with the agent, in an amount sufficient to increase the level or activity of NFkB in the immune cell relative to an immune cell in the absence of contacting the target cell with the agent that induces iron-dependent cellular disassembly.

In some embodiments, the disclosure relates to a method of increasing the level or activity of interferon regulatory factor (IRF) or Stimulator of Interferon Genes (STING) in an immune cell, comprising: (i) contacting a target cell with an agent that induces iron-dependent cellular disassembly and (ii) exposing the immune cell to the target cell that has been contacted with the agent or postcellular signaling factors produced by the target cell that has been contacted with the agent, in an amount sufficient to increase the level or activity of IRF or STING in the immune cell relative to an immune cell in the absence of contacting the target cell with the agent that induces iron-dependent cellular disassembly. In some embodiments, the disclosure relates to a method of increasing the level or activity of a pro-immune cytokine in an immune cell, comprising: (i) contacting a target cell with an agent that induces iron-dependent cellular disassembly and (ii) exposing the immune cell to the target cell that has been contacted with the agent or postcellular signaling factors produced by the target cell that has been contacted with the agent, in an amount sufficient to increase the level or activity of the pro-immune cytokine in the immune cell relative to an immune cell in the absence of contacting the target cell with the agent that induces iron-dependent cellular disassembly.

In some embodiments, the method is carried out in vitro. In some embodiments, the method is carried out ex vivo. In some embodiments, the method is carried out in vivo. In some embodiments, step (i) is carried out in vitro and step (ii) is carried out in vivo.

In some embodiments, the immune cell is a macrophage, monocyte, dendritic cell, T cell, CD4+ cell, CD8+ cell, or CD3+ cell. In some embodiments, the immune cell is a THP-1 cell.

In some embodiments, the disclosure relates to a method of increasing immune activity of an immune cell, comprising contacting the immune cell with a target cell or postcellular signaling factors produced by the target cell, wherein the target cell has been previously contacted with an agent that induces iron-dependent cellular disassembly, in an amount sufficient to increase immune activity of the immune cell relative to an immune cell in the absence of contacting the target cell with the agent that induces iron-dependent cellular disassembly.

In some embodiments, the disclosure relates to a method of increasing the level or activity of NFkB in an immune cell, comprising contacting the immune cell with a target cell or postcellular signaling factors produced by the target cell, wherein the target cell has been previously contacted with an agent that induces iron-dependent cellular disassembly, in an amount sufficient to increase the level or activity of NFkB in the immune cell relative to an immune cell in the absence of contacting the target cell with the agent that induces iron-dependent cellular disassembly.

In some embodiments, the disclosure relates to a method of increasing the level or activity of interferon regulatory factor (IRF) or Stimulator of Interferon Genes (STING) in an immune cell, comprising contacting the immune cell with a target cell or postcellular signaling factors produced by the target cell, wherein the target cell has been previously contacted with an agent that induces iron-dependent cellular disassembly, in an amount sufficient to increase the level or activity of interferon regulatory factor (IRF) or Stimulator of Interferon Genes (STING) in the immune cell relative to an immune cell in the absence of contacting the target cell with the agent that induces iron-dependent cellular disassembly.

In some embodiments, the disclosure relates to a method of increasing the level or activity of a pro-immune cytokine in an immune cell, comprising contacting the immune cell with a target cell or postcellular signaling factors produced by the target cell, wherein the target cell has been previously contacted with an agent that induces iron-dependent cellular disassembly, in an amount sufficient to increase the level or activity of a pro-immune cytokine in the immune cell relative to an immune cell in the absence of contacting the target cell with the agent that induces iron-dependent cellular disassembly.

In some embodiments, the step of contacting the immune cell with the target cell is carried out in vitro. In some embodiments, the step of contacting the immune cell with the target cell is carried out ex vivo. In some embodiments, the step of contacting the immune cell with the target cell is carried out in vivo.

In some embodiments, the target cell was previously contacted with the agent in vitro. In some embodiments, the target cell was previously contacted with the agent ex vivo. In some embodiments, the target cell was previously contacted with the agent in vivo. In some embodiments, the disclosure relates to a method of increasing immune activity of an immune cell in a tissue or subject, comprising contacting a target cell in the tissue or subject with an agent that induces iron-dependent cellular disassembly in an amount sufficient to increase immune activity of the immune cell relative to an immune cell in a tissue or subject in which the target cell is not contacted with the agent that induces iron-dependent cellular disassembly.

In some embodiments, the disclosure relates to a method of increasing the level or activity of NFkB in an immune cell in a tissue or subject, comprising contacting a target cell in the tissue or subject with an agent that induces iron-dependent cellular disassembly in an amount sufficient to increase ithe level or activity of NFkB in the immune cell relative to an immune cell in a tissue or subject in which the target cell is not contacted with the agent that induces iron-dependent cellular disassembly.

In some embodiments, the disclosure relates to a method of increasing the level or activity of IRF or STING in an immune cell in a tissue or subject, comprising contacting a target cell in the tissue or subject with an agent that induces iron-dependent cellular disassembly in an amount sufficient to increase the level or activity of IRF or STING in the immune cell relative to an immune cell in a tissue or subject in which the target cell is not contacted with the agent that induces iron-dependent cellular disassembly.

In some embodiments, the disclosure relates to a method of increasing the level or activity of a pro-immune cytokine in an immune cell in a tissue or subject, comprising contacting a target cell in the tissue or subject with an agent that induces iron-dependent cellular disassembly in an amount sufficient to increase the level or activity of the pro-immune cytokine in the immune cell relative to an immune cell in a tissue or subject in which the target cell is not contacted with the agent that induces iron-dependent cellular disassembly. In some embodiments, the target cell and the immune cell are in close proximity or physical contact in the tissue or subject. In some embodiments, the target cell and the immune cell are present in the same tissue or organ in the subject.

In some aspects, the disclosure relates to a method of increasing the level or activity of NFkB in a cell, tissue or subject, comprising administering to the cell, tissue or subject an agent that induces iron-dependent cellular disassembly in an amount sufficient to increase the level or activity of NFkB relative to a cell, tissue or subject that is not treated with the agent that induces iron-dependent cellular disassembly.

In one embodiment, the subject is in need of an increased level or activity of NFkB. In one embodiment, the level or activity of NFkB is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In some aspects, the disclosure relates to a method of increasing the level or activity of IRF or STING in a cell, tissue or subject, comprising administering to the cell, tissue or subject an agent that induces iron-dependent cellular disassembly in an amount sufficient to increase the level or activity of IRF or STING relative to a cell, tissue or subject that is not treated with the agent that induces iron-dependent cellular disassembly.

In one embodiment, the subject is in need of an increased level or activity of IRF or STING.

In one embodiment, the level or activity of IRF or STING is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In some aspects, the disclosure relates to a method of increasing the level or activity of macrophages, monocytes, T cells and/or dendritic cells in a tissue or subject, comprising administering to the tissue or subject an agent that induces iron-dependent cellular disassembly in an amount sufficient to increase the level or activity of macrophages, monocytes, T cells and/or dendritic cells relative to a tissue or subject that is not treated with the agent that induces iron-dependent cellular disassembly.

In one embodiment, the subject is in need of an increased level or activity of macrophages, monocytes or dendritic cells.

In one embodiment, the level or activity of macrophages, monocytes, T cells or dendritic cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the agent that induces iron-dependent cellular disassembly.

In some aspects, the disclosure relates to a method of increasing the level or activity of CD4+, CD8+, or CD3+ cells in a tissue or subject, comprising administering to the subject an agent that induces iron-dependent cellular disassembly in an amount sufficient to increase the level or activity of CD4+, CD8+, or CD3+ cells relative to a tissue or subject that is not treated with the agent that induces iron-dependent cellular disassembly.

In one embodiment, the subject is in need of an increased level or activity of CD4+, CD8+, or CD3+ cells.

In one embodiment, the level or activity of CD4+, CD8+, or CD3+ cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the agent that induces iron-dependent cellular disassembly.

In some aspects, the disclosure relates to a method of increasing the level or activity of a pro-immune cytokine in a cell, tissue or subject, comprising administering to the cell, tissue or subject an agent that induces iron-dependent cellular disassembly in an amount sufficient to increase the level or activity of the pro-immune cytokine relative to a cell, tissue or subject that is not treated with the agent that induces iron-dependent cellular disassembly.

In one embodiment, the subject is in need of an increased level or activity of a pro-immune cytokine.

In one embodiment, the level or activity of the pro-immune cytokine is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In one embodiment, the pro-immune cytokine is selected from IFN-α, IL-1, IL-12, IL-18, IL-2, IL-15, IL-4, IL-6, TNF-α, IL-17 and GMCSF.

In some embodiments, the methods of the invention further include, before administration of the agent that induces iron-dependent cellular disassembly, evaluating the cell, tissue or subject for one or more of: the level or activity of NFkB; the level or activity of macrophages; the level or activity of monocytes; the level or activity of dendritic cells; the level or activity of CD4+ cells, CD8+ cells, or CD3+ cells; the level or activity of T cells; and the level or activity of a pro-immune cytokine.

In one embodiment, the methods of the invention further include, after administration of the agent that induces iron-dependent cellular disassembly, evaluating the cell, tissue or subject for one or more of: the level or activity of NFkB, IRF or STING; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine.

Methods of measuring the level or activity of NFkB, IRF or STING; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine are known in the art.

For example, the protein level or activity of NFkB, IRF or STING may be measured by suitable techniques known in the art including ELISA, Western blot or in situ hybridization. The level of a nucleic acid (e.g. an mRNA) encoding NFkB, IRF or STING may be measured using suitable techniques known in the art including polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, in situ hybridization, array analysis, deoxyribonucleic acid sequencing, restriction fragment length polymorphism analysis, and combinations or sub-combinations thereof.

Methods for measuring the level and activity of macrophages are described, for example, in Chitu et al., 2011, Curr Protoc Immunol 14: 1-33. The level and activity of monocytes may be measured by flow cytometry, as described, for example, in Henning et al., 2015, Journal of Immunological Methods 423: 78-84. The level and activity of dendritic cells may be measured by flow cytometry, as described, for example in Dixon et al., 2001, Infect Immun. 69(7): 4351-4357. Each of these references is incorporated by reference herein in its entirety.

The level or activity of T cells may be assessed using a human CD4+ T-cell-based proliferative assay. For example, cells are labeled with the fluorescent dye 5,6-carboxyfluorescein diacetate succinimidyl ester (CFSE). Those cells that proliferate show a reduction in CFSE fluorescence intensity, which is measured directly by flow cytometry. Alternatively, radioactive thymidine incorporation can be used to assess the rate of growth of the T cells.

In some embodiments, an increase in immune response may be associated with reduced activation of regulatory T cells (Tregs). Functional activity T regs may be assessed using an in vitro Treg suppression assay. Such an assay is described in Collinson and Vignali (Methods Mol Biol. 2011; 707: 21-37, incorporated by reference in its entirety herein).

The level or activity of a pro-immune cytokine may be quantified, for example, in CD8+ T cells. In embodiments, the pro-immune cytokine is selected from interferon alpha (IFN-α), interleukin-1 (IL-1), IL-12, IL-18, IL-2, IL-15, IL-4, IL-6, tumor necrosis factor alpha (TNF-α), IL-17, and granulocyte-macrophage colony-stimulating factor (GMCSF). Quantitation can be carried out using the ELISPOT (enzyme-linked immunospot) technique, that detects T cells that secrete a given cytokine (e.g. IFN-α) in response to an antigenic stimulation. T cells are cultured with antigen-presenting cells in wells which have been coated with, e.g., anti-IFN-α antibodies. The secreted IFN-α is captured by the coated antibody and then revealed with a second antibody coupled to a chromogenic substrate. Thus, locally secreted cytokine molecules form spots, with each spot corresponding to one IFN-α-secreting cell. The number of spots allows one to determine the frequency of IFN-α-secreting cells specific for a given antigen in the analyzed sample. The ELISPOT assay has also been described for the detection of TNF-α, interleukin-4 (IL-4), IL-6, IL-12, and GMCSF.

### IV. Methods of Treating Disorders

Applicants have shown that treatment of cells with agents that induce iron-dependent cellular disassembly results in the production and release of postcellular signaling factors that increase immune activity. Accordingly, agents that induce iron-dependent cellular disassembly and increase immune activity may be used in the treatment of disorders that may benefit from increased immune activity, such as cancer and infections.

### A. Infectious Diseases

As provided herein, an agent that induces iron-dependent cellular disassembly (e.g., ferroptosis) can activate immune cells (e.g., T cells, B cells, NK cells, etc.) and, therefore, can enhance immune cell functions such as inhibiting bacterial and/or viral infection, and/or restoring immune surveillance and immune memory function to treat infection. Accordingly, in some embodiments, the compositions of the invention, e.g., comprising agents that induce iron-dependent cellular disassembly (e.g., ferroptosis), are used to treat an infection or infectious disease in a subject, for example, a chronic infection.

As used herein, the term “infection” refers to any state in which cells or a tissue of an organism (i.e., a subject) is infected by an infectious agent (e.g., a subject has an intracellular pathogen infection, e.g., a chronic intracellular pathogen infection). As used herein, the term “infectious agent” refers to a foreign biological entity (i.e. a pathogen) in at least one cell of the infected organism. For example, infectious agents include, but are not limited to bacteria, viruses, protozoans, and fungi. Intracellular pathogens are of particular interest. Infectious diseases are disorders caused by infectious agents. Some infectious agents cause no recognizable symptoms or disease under certain conditions, but have the potential to cause symptoms or disease under changed conditions. The subject methods can be used in the treatment of chronic pathogen infections including, but not limited to, viral infections, e.g., retrovirus, lentivirus, hepadna virus, herpes viruses, pox viruses, or human papilloma viruses; intracellular bacterial infections, e.g., Mycobacterium, Chlamydophila, Ehrlichia, Rickettsia, Brucella, Legionella, Francisella, Listeria, Coxiella, Neisseria, Salmonella, Yersinia sp, or Helicobacter pylori; and intracellular protozoan pathogens, e.g., Plasmodium sp, Trypanosoma sp., Giardia sp., Toxoplasma sp., or Leishmania sp.

Infectious diseases that can be treated using the compositions described herein include but are not limited to: HIV, Influenza, Herpes, Giardia, Malaria, Leishmania, pathogenic infection by the virus Hepatitis (A, B, or C), herpes virus (e.g., VZV, HSV-I, HAV-6, HSV-II, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus, pathogenic infection by the bacteria chlamydia, rickettsial bacteria, mycobacteria, staphylococci, streptococci, pneumonococci, meningococci and conococci, klebsiella, proteus, serratia, pseudomonas, E. coli, legionella, diphtheria, salmonella, bacilli, cholera, tetanus, botulism, anthrax, plague, leptospirosis, and Lyme's disease bacteria, pathogenic infection by the fungi Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum, and pathogenic infection by the parasites Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and/or Nippostrongylus brasiliensis.

The term “chronic infection” refers to an infection lasting about one month or more, for example, for at least one month, two months, three months, four months, five months, or six months. In some embodiments, a chronic infection is associated with the increased production of anti-inflammatory chemokines in and/or around the infected area(s). Chronic infections include, but are not limited to, infections by HIV, HPV, Hepatitis B, Hepatitis C, EBV, CMV, M. tuberculosis, and intracellular bacteria and parasites. In some embodiments, the chronic infection is a bacterial infection. In some embodiments, the chronic infection is a viral infection.

### B. Cancer

As provided herein, an agent that induces iron-dependent cellular disassembly (e.g., ferroptosis) can activate immune cells (e.g., T cells, B cells, NK cells, etc.) and, therefore, can enhance immune cell functions such as, for example, that involved in immunotherapies. Accordingly, in certain aspects, the disclosure relates to a method of treating a subject diagnosed with cancer, comprising administering to the subject, in combination (a) an immunotherapeutic anti-neoplastic agent and (b) an agent that induces iron-dependent cellular disassembly, thereby treating the cancer in the subject.

The ability of cancer cells to harness a range of complex, overlapping mechanisms to prevent the immune system from distinguishing self from non-self represents the fundamental mechanism of cancers to evade immunesurveillance. Mechanism(s) include disruption of antigen presentation, disruption of regulatory pathways controlling T cell activation or inhibition (immune checkpoint regulation), recruitment of cells that contribute to immune suppression (Tregs, MDSC) or release of factors that influence immune activity (IDO, PGE2). (See Harris et al., 2013, J Immunotherapy Cancer 1:12; Chen et al., 2013, Immunity 39:1; Pardoll, et al., 2012, Nature Reviews: Cancer 12:252; and Sharma et al., 2015, Cell 161:205, each of which is incorporated by reference herein in its entirety.)

Immune Checkpoint Modulators

In some embodiments, the immunotherapeutic is an immune checkpoint modulator of an immune checkpoint molecule. Examples include LAG-3 (Triebel et al., 1990, J. Exp. Med. 171: 1393-1405), TIM-3 (Sakuishi et al., 2010, J. Exp. Med. 207: 2187-2194) and VISTA (Wang et al., 2011, J. Exp. Med. 208: 577-592). Examples of co-stimulatory molecules that improve immune responses include ICOS (Fan et al., 2014, J. Exp. Med. 211: 715-725), OX40 (Curti et al., 2013, Cancer Res. 73: 7189-7198) and 4-1BB (Melero et al., 1997, Nat. Med. 3: 682-685).

Immune checkpoints may be stimulatory immune checkpoints (i.e. molecules that stimulate the immune response) or inhibitory immune checkpoints (i.e. molecules that inhibit immune response). In some embodiments, the immune checkpoint modulator is an antagonist of an inhibitory immune checkpoint. In some embodiments, the immune checkpoint modulator is an agonist of a stimulatory immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In certain embodiments, the immune checkpoint modulator is capable of binding to, or modulating the activity of more than one immune checkpoint. Examples of stimulatory and inhibitory immune checkpoints, and molecules that modulate these immune checkpoints that may be used in the methods of the invention, are provided below.

i. Stimulatory Immune Checkpoint Molecules

CD27 supports antigen-specific expansion of naïve T cells and is vital for the generation of T cell memory (see, e.g., Hendriks et al. (2000) Nat. Immunol. 171 (5): 433-40). CD27 is also a memory marker of B cells (see, e.g., Agematsu et al. (2000) Histol. Histopathol. 15 (2): 573-6. CD27 activity is governed by the transient availability of its ligand, CD70, on lymphocytes and dendritic cells (see, e.g., Borst et al. (2005) Curr. Opin. Immunol. 17 (3): 275-81). Multiple immune checkpoint modulators specific for CD27 have been developed and may be used as disclosed herein. In some embodiments, the immune checkpoint modulator is an agent that modulates the activity and/or expression of CD27. In some embodiments, the immune checkpoint modulator is an agent that binds to CD27 (e.g., an anti-CD27 antibody). In some embodiments, the checkpoint modulator is a CD27 agonist. In some embodiments, the checkpoint modulator is a CD27 antagonist. In some embodiments, the immune checkpoint modulator is an CD27-binding protein (e.g., an antibody). In some embodiments, the immune checkpoint modulator is varlilumab (Celldex Therapeutics). Additional CD27-binding proteins (e.g., antibodies) are known in the art and are disclosed, e.g., in U.S. Pat. Nos. 9,248,183, 9,102,737, 9,169,325, 9,023,999, 8,481,029; U.S. Patent Application Publication Nos. 2016/0185870, 2015/0337047, 2015/0299330, 2014/0112942, 2013/0336976, 2013/0243795, 2013/0183316, 2012/0213771, 2012/0093805, 2011/0274685, 2010/0173324; and PCT Publication Nos. WO 2015/016718, WO 2014/140374, WO 2013/138586, WO 2012/004367, WO 2011/130434, WO 2010/001908, and WO 2008/051424, each of which is incorporated by reference herein.

CD28. Cluster of Differentiation 28 (CD28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). Binding with its two ligands, CD80 and CD86, expressed on dendritic cells, prompts T cell expansion (see, e.g., Prasad et al. (1994) Proc. Nat'l. Acad. Sci. USA 91(7): 2834-8). Multiple immune checkpoint modulators specific for CD28 have been developed and may be used as disclosed herein. In some embodiments, the immune checkpoint modulator is an agent that modulates the activity and/or expression of CD28. In some embodiments, the immune checkpoint modulator is an agent that binds to CD28 (e.g., an anti-CD28 antibody). In some embodiments, the checkpoint modulator is an CD28 agonist. In some embodiments, the checkpoint modulator is an CD28 antagonist. In some embodiments, the immune checkpoint modulator is an CD28-binding protein (e.g., an antibody). In some embodiments, the immune checkpoint modulator is selected from the group consisting of TABO8 (TheraMab LLC), lulizumab (also known as BMS-931699, Bristol-Myers Squibb), and FR104 (OSE Immunotherapeutics). Additional CD28-binding proteins (e.g., antibodies) are known in the art and are disclosed, e.g., in U.S. Pat. Nos. 9,119,840, 8,709,414, 9,085,629, 8,034,585, 7,939,638, 8,389,016, 7,585,960, 8,454,959, 8,168,759, 8,785,604, 7,723,482; U.S. Patent Application Publication Nos. 2016/0017039, 2015/0299321, 2015/0150968, 2015/0071916, 2015/0376278, 2013/0078257, 2013/0230540, 2013/0078236, 2013/0109846, 2013/0266577, 2012/0201814, 2012/0082683, 2012/0219553, 2011/0189735, 2011/0097339, 2010/0266605, 2010/0168400, 2009/0246204, 2008/0038273; and PCT Publication Nos. WO 2015198147, WO 2016/05421, WO 2014/1209168, WO 2011/101791, WO 2010/007376, WO 2010/009391, WO 2004/004768, WO 2002/030459, WO 2002/051871, and WO 2002/047721, each of which is incorporated by reference herein.

CD40. Cluster of Differentiation 40 (CD40, also known as TNFRSFS) is found on a variety of immune system cells including antigen presenting cells. CD40L, otherwise known as CD154, is the ligand of CD40 and is transiently expressed on the surface of activated CD4⁺ T cells. CD40 signaling is known to ‘license’ dendritic cells to mature and thereby trigger T-cell activation and differentiation (see, e.g., O'Sullivan et al. (2003) Crit. Rev. Immunol. 23 (1): 83-107. Multiple immune checkpoint modulators specific for CD40 have been developed and may be used as disclosed herein. In some embodiments, the immune checkpoint modulator is an agent that modulates the activity and/or expression of CD40. In some embodiments, the immune checkpoint modulator is an agent that binds to CD40 (e.g., an anti-CD40 antibody). In some embodiments, the checkpoint modulator is a CD40 agonist. In some embodiments, the checkpoint modulator is an CD40 antagonist. In some embodiments, the immune checkpoint modulator is a CD40-binding protein selected from the group consisting of dacetuzumab (Genentech/Seattle Genetics), CP-870,893 (Pfizer), bleselumab (Astellas Pharma), lucatumumab (Novartis), CFZ533 (Novartis; see, e.g., Cordoba et al. (2015) Am. J. Transplant. 15(11): 2825-36), RG7876 (Genentech Inc.), FFP104 (PanGenetics, B.V.), APX005 (Apexigen), BI 655064 (Boehringer Ingelheim), Chi Lob 7/4 (Cancer Research UK; see, e.g., Johnson et al. (2015) Clin. Cancer Res. 21(6): 1321-8), ADC-1013 (BioInvent International), SEA-CD40 (Seattle Genetics), XmAb 5485 (Xencor), PG120 (PanGenetics B.V.), teneliximab (Bristol-Myers Squibb; see, e.g., Thompson et al. (2011) Am. J. Transplant. 11(5): 947-57), and AKH3 (Biogen; see, e.g., International Publication No. WO 2016/028810). Additional CD40-binding proteins (e.g., antibodies) are known in the art and are disclosed, e.g., in U.S. Pat. Nos. 9,234,044, 9,266,956, 9,109,011, 9,090,696, 9,023,360, 9,023,361, 9,221,913, 8,945,564, 8,926,979, 8,828,396, 8,637,032, 8,277,810, 8,088,383, 7,820,170, 7,790,166, 7,445,780, 7,361,345, 8,961,991, 8,669,352, 8,957,193, 8,778,345, 8,591,900, 8,551,485, 8,492,531, 8,362,210, 8,388,971; U.S. Patent Application Publication Nos. 2016/0045597, 2016/0152713, 2016/0075792, 2015/0299329, 2015/0057437 2015/0315282, 2015/0307616, 2014/0099317, 2014/0179907, 2014/0349395, 2014/0234344, 2014/0348836, 2014/0193405, 2014/0120103, 2014/0105907, 2014/0248266, 2014/0093497, 2014/0010812, 2013/0024956, 2013/0023047, 2013/0315900, 2012/0087927, 2012/0263732, 2012/0301488, 2011/0027276, 2011/0104182, 2010/0234578, 2009/0304687, 2009/0181015, 2009/0130715, 2009/0311254, 2008/0199471, 2008/0085531, 2016/0152721, 2015/0110783, 2015/0086991, 2015/0086559, 2014/0341898, 2014/0205602, 2014/0004131, 2013/0011405, 2012/0121585, 2011/0033456, 2011/0002934, 2010/0172912, 2009/0081242, 2009/0130095, 2008/0254026, 2008/0075727, 2009/0304706, 2009/0202531, 2009/0117111, 2009/0041773, 2008/0274118, 2008/0057070, 2007/0098717, 2007/0218060, 2007/0098718, 2007/0110754; and PCT Publication Nos. WO 2016/069919, WO 2016/023960, WO 2016/023875, WO 2016/028810, WO 2015/134988, WO 2015/091853, WO 2015/091655, WO 2014/065403, WO 2014/070934, WO 2014/065402, WO 2014/207064, WO 2013/034904, WO 2012/125569, WO 2012/149356, WO 2012/111762, WO 2012/145673, WO 2011/123489, WO 2010/123012, WO 2010/104761, WO 2009/094391, WO 2008/091954, WO 2007/129895, WO 2006/128103, WO 2005/063289, WO 2005/063981, WO 2003/040170, WO 2002/011763, WO 2000/075348, WO 2013/164789, WO 2012/075111, WO 2012/065950, WO 2009/062054, WO 2007/124299, WO 2007/053661, WO 2007/053767, WO 2005/044294, WO 2005/044304, WO 2005/044306, WO 2005/044855, WO 2005/044854, WO 2005/044305, WO 2003/045978, WO 2003/029296, WO 2002/028481, WO 2002/028480, WO 2002/028904, WO 2002/028905, WO 2002/088186, and WO 2001/024823, each of which is incorporated by reference herein.

CD122. CD122 is the Interleukin-2 receptor beta sub-unit and is known to increase proliferation of CD8⁺ effector T cells. See, e.g., Boyman et al. (2012) Nat. Rev. Immunol. 12 (3): 180-190. Multiple immune checkpoint modulators specific for CD122 have been developed and may be used as disclosed herein. In some embodiments, the immune checkpoint modulator is an agent that modulates the activity and/or expression of CD122. In some embodiments, the immune checkpoint modulator is an agent that binds to CD122 (e.g., an anti-CD122 antibody). In some embodiments, the checkpoint modulator is an CD122 agonist. In some embodiments, the checkpoint modulator is an CD22 agonist. In some embodiments, the immune checkpoint modulator is humanized MiK-Beta-1 (Roche; see, e.g., Morris et al. (2006) Proc Nat'l. Acad. Sci. USA 103(2): 401-6, which is incorporated by reference). Additional CD122-binding proteins (e.g., antibodies) are known in the art and are disclosed, e.g., in U.S. Pat. No. 9,028,830, which is incorporated by reference herein.

OX40. The OX40 receptor (also known as CD134) promotes the expansion of effector and memory T cells. OX40 also suppresses the differentiation and activity of T-regulatory cells, and regulates cytokine production (see, e.g., Croft et al. (2009) Immunol. Rev. 229(1): 173-91). Multiple immune checkpoint modulators specific for OX40 have been developed and may be used as disclosed herein. In some embodiments, the immune checkpoint modulator is an agent that modulates the activity and/or expression of OX40. In some embodiments, the immune checkpoint modulator is an agent that binds to OX40 (e.g., an anti-OX40 antibody). In some embodiments, the checkpoint modulator is an OX40 agonist. In some embodiments, the checkpoint modulator is an OX40 antagonist. In some embodiments, the immune checkpoint modulator is a OX40-binding protein (e.g., an antibody) selected from the group consisting of MEDI6469 (AgonOx/Medimmune), pogalizumab (also known as MOXR0916 and RG7888; Genentech, Inc.), tavolixizumab (also known as MEDI0562; Medimmune), and GSK3174998 (GlaxoSmithKline). Additional OX-40-binding proteins (e.g., antibodies) are known in the art and are disclosed, e.g., in U.S. Pat. Nos. 9,163,085, 9,040,048, 9,006,396, 8,748,585, 8,614,295, 8,551,477, 8,283,450, 7,550,140; U.S. Patent Application Publication Nos. 2016/0068604, 2016/0031974, 2015/0315281, 2015/0132288, 2014/0308276, 2014/0377284, 2014/0044703, 2014/0294824, 2013/0330344, 2013/0280275, 2013/0243772, 2013/0183315, 2012/0269825, 2012/0244076, 2011/0008368, 2011/0123552, 2010/0254978, 2010/0196359, 2006/0281072; and PCT Publication Nos. WO 2014/148895, WO 2013/068563, WO 2013/038191, WO 2013/028231, WO 2010/096418, WO 2007/062245, and WO 2003/106498, each of which is incorporated by reference herein.

GITR. Glucocorticoid-induced TNFR family related gene (GITR) is a member of the tumor necrosis factor receptor (TNFR) superfamily that is constitutively or conditionally expressed on Treg, CD4, and CD8 T cells. GITR is rapidly upregulated on effector T cells following TCR ligation and activation. The human GITR ligand (GITRL) is constitutively expressed on APCs in secondary lymphoid organs and some nonlymphoid tissues. The downstream effect of GITR:GITRL interaction induces attenuation of Treg activity and enhances CD4⁺ T cell activity, resulting in a reversal of Treg-mediated immunosuppression and increased immune stimulation. Multiple immune checkpoint modulators specific for GITR have been developed and may be used as disclosed herein. In some embodiments, the immune checkpoint modulator is an agent that modulates the activity and/or expression of GITR. In some embodiments, the immune checkpoint modulator is an agent that binds to GITR (e.g., an anti-GITR antibody). In some embodiments, the checkpoint modulator is an GITR agonist. In some embodiments, the checkpoint modulator is an GITR antagonist. In some embodiments, the immune checkpoint modulator is a GITR-binding protein (e.g., an antibody) selected from the group consisting of TRX518 (Leap Therapeutics), MK-4166 (Merck & Co.), MEDI-1873 (MedImmune), INCAGN1876 (Agenus/Incyte), and FPA154 (Five Prime Therapeutics). Additional GITR-binding proteins (e.g., antibodies) are known in the art and are disclosed, e.g., in U.S. Pat. Nos. 9,309,321, 9,255,152, 9,255,151, 9,228,016, 9,028,823, 8,709,424, 8,388,967; U.S. Patent Application Publication Nos. 2016/0145342, 2015/0353637, 2015/0064204, 2014/0348841, 2014/0065152, 2014/0072566, 2014/0072565, 2013/0183321, 2013/0108641, 2012/0189639; and PCT Publication Nos. WO 2016/054638, WO 2016/057841, WO 2016/057846, WO 2015/187835, WO 2015/184099, WO 2015/031667, WO 2011/028683, and WO 2004/107618, each of which is incorporated by reference herein.

ICOS. Inducible T-cell costimulator (ICOS, also known as CD278) is expressed on activated T cells. Its ligand is ICOSL, which is expressed mainly on B cells and dendritic cells. ICOS is important in T cell effector function. ICOS expression is up-regulated upon T cell activation (see, e.g., Fan et al. (2014) J. Exp. Med. 211(4): 715-25). Multiple immune checkpoint modulators specific for ICOS have been developed and may be used as disclosed herein. In some embodiments, the immune checkpoint modulator is an agent that modulates the activity and/or expression of ICOS. In some embodiments, the immune checkpoint modulator is an agent that binds to ICOS (e.g., an anti-ICOS antibody). In some embodiments, the checkpoint modulator is an ICOS agonist. In some embodiments, the checkpoint modulator is an ICOS antagonist. In some embodiments, the immune checkpoint modulator is a ICOS-binding protein (e.g., an antibody) selected from the group consisting of MEDI-570 (also known as JMab-136, Medimmune), GSK3359609 (GlaxoSmithKline/INSERM), and JTX-2011 (Jounce Therapeutics). Additional ICOS-binding proteins (e.g., antibodies) are known in the art and are disclosed, e.g., in U.S. Pat. Nos. 9,376,493, 7,998,478, 7,465,445, 7,465,444; U.S. Patent Application Publication Nos. 2015/0239978, 2012/0039874, 2008/0199466, 2008/0279851; and PCT Publication No. WO 2001/087981, each of which is incorporated by reference herein.

4-1BB. 4-1BB (also known as CD137) is a member of the tumor necrosis factor (TNF) receptor superfamily. 4-1BB (CD137) is a type II transmembrane glycoprotein that is inducibly expressed on primed CD4⁺ and CD8⁺ T cells, activated NK cells, DCs, and neutrophils, and acts as a T cell costimulatory molecule when bound to the 4-1BB ligand (4-1BBL) found on activated macrophages, B cells, and DCs. Ligation of the 4-1BB receptor leads to activation of the NF-κB, c-Jun and p38 signaling pathways and has been shown to promote survival of CD8⁺ T cells, specifically, by upregulating expression of the antiapoptotic genes BcL-x(L) and Bfl-1. In this manner, 4-1BB serves to boost or even salvage a suboptimal immune response. Multiple immune checkpoint modulators specific for 4-1BB have been developed and may be used as disclosed herein. In some embodiments, the immune checkpoint modulator is an agent that modulates the activity and/or expression of 4-1BB. In some embodiments, the immune checkpoint modulator is an agent that binds to 4-1BB (e.g., an anti-4-1BB antibody). In some embodiments, the checkpoint modulator is an 4-1BB agonist. In some embodiments, the checkpoint modulator is an 4-1BB antagonist. In some embodiments, the immune checkpoint modulator is a 4-1BB-binding protein is urelumab (also known as BMS-663513; Bristol-Myers Squibb) or utomilumab (Pfizer). In some embodiments, the immune checkpoint modulator is a 4-1BB-binding protein (e.g., an antibody). 4-1BB-binding proteins (e.g., antibodies) are known in the art and are disclosed, e.g., in U.S. Pat. Nos. 9,382,328, 8,716,452, 8,475,790, 8,137,667, 7,829,088, 7,659,384; U.S. Patent Application Publication Nos. 2016/0083474, 2016/0152722, 2014/0193422, 2014/0178368, 2013/0149301, 2012/0237498, 2012/0141494, 2012/0076722, 2011/0177104, 2011/0189189, 2010/0183621, 2009/0068192, 2009/0041763, 2008/0305113, 2008/0008716; and PCT Publication Nos. WO 2016/029073, WO 2015/188047, WO 2015/179236, WO 2015/119923, WO 2012/032433, WO 2012/145183, WO 2011/031063, WO 2010/132389, WO 2010/042433, WO 2006/126835, WO 2005/035584, WO 2004/010947; and Martinez-Forero et al. (2013) J. Immunol. 190(12): 6694-706, and Dubrot et al. (2010) Cancer Immunol. Immunother. 59(8): 1223-33, each of which is incorporated by reference herein.

ii. Inhibitory Immune Checkpoint Molecules

ADORA2A. The adenosine A2A receptor (A2A4) is a member of the G protein-coupled receptor (GPCR) family which possess seven transmembrane alpha helices, and is regarded as an important checkpoint in cancer therapy. A2A receptor can negatively regulate overreactive immune cells (see, e.g., Ohta et al. (2001) Nature 414(6866): 916-20). Multiple immune checkpoint modulators specific for ADORA2A have been developed and may be used as disclosed herein. In some embodiments, the immune checkpoint modulator is an agent that modulates the activity and/or expression of ADORA2A. In some embodiments, the immune checkpoint modulator is an agent that binds to ADORA2A (e.g., an anti-ADORA2A antibody). In some embodiments, the immune checkpoint modulator is a ADORA2A-binding protein (e.g., an antibody). In some embodiments, the checkpoint modulator is an ADORA2A agonist. In some embodiments, the checkpoint modulator is an ADORA2A antagonist. ADORA2A-binding proteins (e.g., antibodies) are known in the art and are disclosed, e.g., in U.S. Patent Application Publication No. 2014/0322236, which is incorporated by reference herein.

B7-H3. B7-H3 (also known as CD276) belongs to the B7 superfamily, a group of molecules that costimulate or down-modulate T-cell responses. B7-H3 potently and consistently down-modulates human T-cell responses (see, e.g., Leitner et al. (2009) Eur. J. Immunol. 39(7): 1754-64). Multiple immune checkpoint modulators specific for B7-H3 have been developed and may be used as disclosed herein. In some embodiments, the immune checkpoint modulator is an agent that modulates the activity and/or expression of B7-H3. In some embodiments, the immune checkpoint modulator is an agent that binds to B7-H3 (e.g., an anti-B7-H3 antibody). In some embodiments, the checkpoint modulator is an B7-H3 agonist. In some embodiments, the checkpoint modulator is an B7-H3 antagonist. In some embodiments, the immune checkpoint modulator is an anti-B7-H3-binding protein selected from the group consisting of DS-5573 (Daiichi Sankyo, Inc.), enoblituzumab (MacroGenics, Inc.), and 8H9 (Sloan Kettering Institute for Cancer Research; see, e.g., Ahmed et al. (2015) J. Biol. Chem. 290(50): 30018-29). In some embodiments, the immune checkpoint modulator is a B7-H3-binding protein (e.g., an antibody). B7-H3-binding proteins (e.g., antibodies) are known in the art and are disclosed, e.g., in U.S. Pat. Nos. 9,371,395, 9,150,656, 9,062,110, 8,802,091, 8,501,471, 8,414,892; U.S. Patent Application Publication Nos. 2015/0352224, 2015/0297748, 2015/0259434, 2015/0274838, 2014/032875, 2014/0161814, 2013/0287798, 2013/0078234, 2013/0149236, 2012/02947960, 2010/0143245, 2002/0102264; PCT Publication Nos. WO 2016/106004, WO 2016/033225, WO 2015/181267, WO 2014/057687, WO 2012/147713, WO 2011/109400, WO 2008/116219, WO 2003/075846, WO 2002/032375; and Shi et al. (2016) Mol. Med. Rep. 14(1): 943-8, each of which is incorporated by reference herein.

B7-H4. B7-H4 (also known as O8E, OV064, and V-set domain-containing T-cell activation inhibitor (VTCN1)), belongs to the B7 superfamily. By arresting cell cycle, B7-H4 ligation of T cells has a profound inhibitory effect on the growth, cytokine secretion, and development of cytotoxicity. Administration of B7-H4Ig into mice impairs antigen-specific T cell responses, whereas blockade of endogenous B7-H4 by specific monoclonal antibody promotes T cell responses (see, e.g., Sica et al. (2003) Immunity 18(6): 849-61). Multiple immune checkpoint modulators specific for B7-H4 have been developed and may be used as disclosed herein. In some embodiments, the immune checkpoint modulator is an agent that modulates the activity and/or expression of B7-H4. In some embodiments, the immune checkpoint modulator is an agent that binds to B7-H4 (e.g., an anti-B7-H4 antibody). In some embodiments, the immune checkpoint modulator is a B7-H4-binding protein (e.g., an antibody). In some embodiments, the checkpoint modulator is an B7-H4 agonist. In some embodiments, the checkpoint modulator is an B7-H4 antagonist. B7-H4-binding proteins (e.g., antibodies) are known in the art and are disclosed, e.g., in U.S. Pat. Nos. 9,296,822, 8,609,816, 8,759,490, 8,323,645; U.S. Patent Application Publication Nos. 2016/0159910, 2016/0017040, 2016/0168249, 2015/0315275, 2014/0134180, 2014/0322129, 2014/0356364, 2014/0328751, 2014/0294861, 2014/0308259, 2013/0058864, 2011/0085970, 2009/0074660, 2009/0208489; and PCT Publication Nos. WO 2016/040724, WO 2016/070001, WO 2014/159835, WO 2014/100483, WO 2014/100439, WO 2013/067492, WO 2013/025779, WO 2009/073533, WO 2007/067991, and WO 2006/104677, each of which is incorporated by reference herein.

BTLA. B and T Lymphocyte Attenuator (BTLA), also known as CD272, has HVEM (Herpesvirus Entry Mediator) as its ligand. Surface expression of BTLA is gradually downregulated during differentiation of human CD8⁺ T cells from the naive to effector cell phenotype, however tumor-specific human CD8⁺ T cells express high levels of BTLA (see, e.g., Derre et al. (2010) J. Clin. Invest. 120 (1): 157-67). Multiple immune checkpoint modulators specific for BTLA have been developed and may be used as disclosed herein. In some embodiments, the immune checkpoint modulator is an agent that modulates the activity and/or expression of BTLA. In some embodiments, the immune checkpoint modulator is an agent that binds to BTLA (e.g., an anti-BTLA antibody). In some embodiments, the immune checkpoint modulator is a BTLA-binding protein (e.g., an antibody). In some embodiments, the checkpoint modulator is an BTLA agonist. In some embodiments, the checkpoint modulator is an BTLA antagonist. BTLA-binding proteins (e.g., antibodies) are known in the art and are disclosed, e.g., in U.S. Pat. Nos. 9,346,882, 8,580,259, 8,563,694, 8,247,537; U.S. Patent Application Publication Nos. 2014/0017255, 2012/0288500, 2012/0183565, 2010/0172900; and PCT Publication Nos. WO 2011/014438, and WO 2008/076560, each of which is incorporated by reference herein.

CTLA-4. Cytotoxic T lymphocyte antigen-4 (CTLA-4) is a member of the immune regulatory CD28-B7 immunoglobulin superfamily and acts on naïve and resting T lymphocytes to promote immunosuppression through both B7-dependent and B7-independent pathways (see, e.g., Kim et al. (2016) J. Immunol. Res., Article ID 4683607, 14 pp.). CTLA-4 is also known as called CD152. CTLA-4 modulates the threshold for T cell activation. See, e.g., Gajewski et al. (2001) J. Immunol. 166(6): 3900-7. Multiple immune checkpoint modulators specific for CTLA-4 have been developed and may be used as disclosed herein. In some embodiments, the immune checkpoint modulator is an agent that modulates the activity and/or expression of CTLA-4. In some embodiments, the immune checkpoint modulator is an agent that binds to CTLA-4 (e.g., an anti-CTLA-4 antibody). In some embodiments, the checkpoint modulator is an CTLA-4 agonist. In some embodiments, the checkpoint modulator is an CTLA-4 antagonist. In some embodiments, the immune checkpoint modulator is a CTLA-4-binding protein (e.g., an antibody) selected from the group consisting of ipilimumab (Yervoy; Medarex/Bristol-Myers Squibb), tremelimumab (formerly ticilimumab; Pfizer/AstraZeneca), JMW-3B3 (University of Aberdeen), and AGEN1884 (Agenus). Additional CTLA-4 binding proteins (e.g., antibodies) are known in the art and are disclosed, e.g., in U.S. Pat. No. 8,697,845; U.S. Patent Application Publication Nos. 2014/0105914, 2013/0267688, 2012/0107320, 2009/0123477; and PCT Publication Nos. WO 2014/207064, WO 2012/120125, WO 2016/015675, WO 2010/097597, WO 2006/066568, and WO 2001/054732, each of which is incorporated by reference herein.

IDO. Indoleamine 2,3-dioxygenase (IDO) is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumor angiogenesis. Prendergast et al., 2014, Cancer Immunol Immunother. 63 (7): 721-35, which is incorporated by reference herein.

Multiple immune checkpoint modulators specific for IDO have been developed and may be used as disclosed herein. In some embodiments, the immune checkpoint modulator is an agent that modulates the activity and/or expression of IDO. In some embodiments, the immune checkpoint modulator is an agent that binds to IDO (e.g., an IDO binding protein, such as an anti-IDO antibody). In some embodiments, the checkpoint modulator is an IDO agonist. In some embodiments, the checkpoint modulator is an IDO antagonist. In some embodiments, the immune checkpoint modulator is selected from the group consisting of Norharmane, Rosmarinic acid, COX-2 inhibitors, alpha-methyl-tryptophan, and Epacadostat. In one embodiment, the modulator is Epacadostat.

KIR. Killer immunoglobulin-like receptors (KIRs) comprise a diverse repertoire of MHCI binding molecules that negatively regulate natural killer (NK) cell function to protect cells from NK-mediated cell lysis. KIRs are generally expressed on NK cells but have also been detected on tumor specific CTLs. Multiple immune checkpoint modulators specific for KIR have been developed and may be used as disclosed herein. In some embodiments, the immune checkpoint modulator is an agent that modulates the activity and/or expression of KIR. In some embodiments, the immune checkpoint modulator is an agent that binds to KIR (e.g., an anti-KIR antibody). In some embodiments, the immune checkpoint modulator is a KIR-binding protein (e.g., an antibody). In some embodiments, the checkpoint modulator is an KIR agonist. In some embodiments, the checkpoint modulator is an KIR antagonist. In some embodiments the immune checkpoint modulator is lirilumab (also known as BMS-986015; Bristol-Myers Squibb). Additional KIR binding proteins (e.g., antibodies) are known in the art and are disclosed, e.g., in U.S. Pat. Nos. 8,981,065, 9,018,366, 9,067,997, 8,709,411, 8,637,258, 8,614,307, 8,551,483, 8,388,970, 8,119,775; U.S. Patent Application Publication Nos. 2015/0344576, 2015/0376275, 2016/0046712, 2015/0191547, 2015/0290316, 2015/0283234, 2015/0197569, 2014/0193430, 2013/0143269, 2013/0287770, 2012/0208237, 2011/0293627, 2009/0081240, 2010/0189723; and PCT Publication Nos. WO 2016/069589, WO 2015/069785, WO 2014/066532, WO 2014/055648, WO 2012/160448, WO 2012/071411, WO 2010/065939, WO 2008/084106, WO 2006/072625, WO 2006/072626, and WO 2006/003179, each of which is incorporated by reference herein.

LAG-3, Lymphocyte-activation gene 3 (LAG-3, also known as CD223) is a CD4-related transmembrane protein that competitively binds MHC II and acts as a co-inhibitory checkpoint for T cell activation (see, e.g., Goldberg and Drake (2011) Curr. Top. Microbiol. Immunol. 344: 269-78). Multiple immune checkpoint modulators specific for LAG-3 have been developed and may be used as disclosed herein. In some embodiments, the immune checkpoint modulator is an agent that modulates the activity and/or expression of LAG-3. In some embodiments, the immune checkpoint modulator is an agent that binds to LAG-3 (e.g., an anti-PD-1 antibody). In some embodiments, the checkpoint modulator is an LAG-3 agonist. In some embodiments, the checkpoint modulator is an LAG-3 antagonist. In some embodiments, the immune checkpoint modulator is a LAG-3-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-1. Programmed cell death protein 1 (PD-1, also known as CD279 and PDCD1) is an inhibitory receptor that negatively regulates the immune system. In contrast to CTLA-4 which mainly affects naïve T cells, PD-1 is more broadly expressed on immune cells and regulates mature T cell activity in peripheral tissues and in the tumor microenvironment. PD-1 inhibits T cell responses by interfering with T cell receptor signaling. PD-1 has two ligands, PD-L1 and PD-L2. Multiple immune checkpoint modulators specific for PD-1 have been developed and may be used as disclosed herein. In some embodiments, the immune checkpoint modulator is an agent that modulates the activity and/or expression of PD-1. In some embodiments, the immune checkpoint modulator is an agent that binds to PD-1 (e.g., an anti-PD-1 antibody). In some embodiments, the checkpoint modulator is an PD-1 agonist. In some embodiments, the checkpoint modulator is an PD-1 antagonist. In some embodiments, the immune checkpoint modulator is a PD-1-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-L1/PD-L2. PD ligand 1 (PD-L1, also knows as B7-H1) and PD ligand 2 (PD-L2, also known as PDCD1LG2, CD273, and B7-DC) bind to the PD-1 receptor. Both ligands belong to the same B7 family as the B7-1 and B7-2 proteins that interact with CD28 and CTLA-4. PD-L1 can be expressed on many cell types including, for example, epithelial cells, endothelial cells, and immune cells. Ligation of PDL-1 decreases IFNγ, TNFα, and IL-2 production and stimulates production of IL10, an anti-inflammatory cytokine associated with decreased T cell reactivity and proliferation as well as antigen-specific T cell anergy. PDL-2 is predominantly expressed on antigen presenting cells (APCs). PDL2 ligation also results in T cell suppression, but where PDL-1-PD-1 interactions inhibits proliferation via cell cycle arrest in the G1/G2 phase, PDL2-PD-1 engagement has been shown to inhibit TCR-mediated signaling by blocking B7:CD28 signals at low antigen concentrations and reducing cytokine production at high antigen concentrations. Multiple immune checkpoint modulators specific for PD-L1 and PD-L2 have been developed and may be used as disclosed herein.

In some embodiments, the immune checkpoint modulator is an agent that modulates the activity and/or expression of PD-L1. In some embodiments, the immune checkpoint modulator is an agent that binds to PD-L1 (e.g., an anti-PD-L1 antibody). In some embodiments, the checkpoint modulator is an PD-L1 agonist. In some embodiments, the checkpoint modulator is an PD-L1 antagonist. In some embodiments, the immune checkpoint modulator is a PD-L1-binding protein (e.g., an antibody or a Fc-fusion protein) selected from the group consisting of durvalumab (also known as MEDI-4736; AstraZeneca/Celgene Corp./Medimmune), atezolizumab (Tecentriq; also known as MPDL3280A and RG7446; Genetech Inc.), avelumab (also known as MSB0010718C; Merck Serono/AstraZeneca); MDX-1105 (Medarex/Bristol-Meyers Squibb), AMP-224 (Amplimmune, GlaxoSmithKline), LY3300054 (Eli Lilly and Co.). Additional PD-L1-binding proteins are known in the art and are disclosed, e.g., in U.S. Patent Application Publication Nos. 2016/0084839, 2015/0355184, 2016/0175397, and PCT Publication Nos. WO 2014/100079, WO 2016/030350, WO2013181634, each of which is incorporated by reference herein.

In some embodiments, the immune checkpoint modulator is an agent that modulates the activity and/or expression of PD-L2. In some embodiments, the immune checkpoint modulator is an agent that binds to PD-L2 (e.g., an anti-PD-L2 antibody). In some embodiments, the checkpoint modulator is an PD-L2 agonist. In some embodiments, the checkpoint modulator is an PD-L2 antagonist. PD-L2-binding proteins (e.g., antibodies) are known in the art and are disclosed, e.g., in U.S. Pat. Nos. 9,255,147, 8,188,238; U.S. Patent Application Publication Nos. 2016/0122431, 2013/0243752, 2010/0278816, 2016/0137731, 2015/0197571, 2013/0291136, 2011/0271358; and PCT Publication Nos. WO 2014/022758, and WO 2010/036959, each of which is incorporated by reference herein.

TIM-3. T cell immunoglobulin mucin 3 (TIM-3, also known as Hepatitis A virus cellular receptor (HAVCR2)) is a A type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Tim-3 was originally identified as being selectively expressed on IFN-γ-secreting Th1 and Tc1 cells (Monney et al. (2002) Nature 415: 536-41). Binding of Gal-9 by the TIM-3 receptor triggers downstream signaling to negatively regulate T cell survival and function. Multiple immune checkpoint modulators specific for TIM-3 have been developed and may be used as disclosed herein. In some embodiments, the immune checkpoint modulator is an agent that modulates the activity and/or expression of TIM-3. In some embodiments, the immune checkpoint modulator is an agent that binds to TIM-3 (e.g., an anti-TIM-3 antibody). In some embodiments, the checkpoint modulator is an TIM-3 agonist. In some embodiments, the checkpoint modulator is an TIM-3 antagonist. In some embodiments, the immune checkpoint modulator is an anti-TIM-3 antibody selected from the group consisting of TSR-022 (AnaptysBio/Tesaro, Inc.) and MGB453 (Novartis). Additional TIM-3 binding proteins (e.g., antibodies) are known in the art and are disclosed, e.g., in U.S. Pat. Nos. 9,103,832, 8,552,156, 8,647,623, 8,841,418; U.S. Patent Application Publication Nos. 2016/0200815, 2015/0284468, 2014/0134639, 2014/0044728, 2012/0189617, 2015/0086574, 2013/0022623; and PCT Publication Nos. WO 2016/068802, WO 2016/068803, WO 2016/071448, WO 2011/155607, and WO 2013/006490, each of which is incorporated by reference herein.

VISTA. V-domain Ig suppressor of T cell activation (VISTA, also known as Platelet receptor Gi24) is an Ig super-family ligand that negatively regulates T cell responses. See, e.g., Wang et al., 2011, J. Exp. Med. 208: 577-92. VISTA expressed on APCs directly suppresses CD4⁺ and CD8⁺ T cell proliferation and cytokine production (Wang et al. (2010) J Exp Med. 208(3): 577-92). Multiple immune checkpoint modulators specific for VISTA have been developed and may be used as disclosed herein. In some embodiments, the immune checkpoint modulator is an agent that modulates the activity and/or expression of VISTA. In some embodiments, the immune checkpoint modulator is an agent that binds to VISTA (e.g., an anti-VISTA antibody). In some embodiments, the checkpoint modulator is an VISTA agonist. In some embodiments, the checkpoint modulator is an VISTA antagonist. In some embodiments, the immune checkpoint modulator is a VISTA-binding protein (e.g., an antibody) selected from the group consisting of TSR-022 (AnaptysBio/Tesaro, Inc.) and MGB453 (Novartis). VISTA-binding proteins (e.g., antibodies) are known in the art and are disclosed, e.g., in U.S. Patent Application Publication Nos. 2016/0096891, 2016/0096891; and PCT Publication Nos. WO 2014/190356, WO 2014/197849, WO 2014/190356 and WO 2016/094837, each of which is incorporated by reference herein.

Additional immunotherapeutics that may be used in the methods disclosed herein include, but are not limited to, Toll-like receptor (TLR) agonists, cell-based therapies, cytokines and cancer vaccines.

TLR Agonists

TLRs are single membrane-spanning non-catalytic receptors that recognize structurally conserved molecules derived from microbes. TLRs together with the Interleukin-1 receptor form a receptor superfamily, known as the “Interleukin-1 Receptor/Toll-Like Receptor Superfamily.” Members of this family are characterized structurally by an extracellular leucine-rich repeat (LRR) domain, a conserved pattern of juxtamembrane cysteine residues, and an intracytoplasmic signaling domain that forms a platform for downstream signaling by recruiting TIR domain-containing adapters including MyD88, TIR domain-containing adaptor (TRAP), and TIR domain-containing adaptor inducing IFNβ (TRIF) (O'Neill et al., 2007, Nat Rev Immunol 7, 353).

The TLRs include TLR1, TLR2, TLR3, TLR4, TLR5, TLR6, TLR7, TLR8, TLR9, and TLR10. TLR2 mediates cellular responses to a large number of microbial products including peptidoglycan, bacterial lipopeptides, lipoteichoic acid, mycobacterial lipoarabinomannan and yeast cell wall components. TLR4 is a transmembrane protein which belongs to the pattern recognition receptor (PRR) family. Its activation leads to an intracellular signaling pathway NF-κB and inflammatory cytokine production which is responsible for activating the innate immune system. TLR5 is known to recognize bacterial flagellin from invading mobile bacteria, and has been shown to be involved in the onset of many diseases, including inflammatory bowel disease.

TLR agonists are known in the art and are described, for example, in US2014/0030294, which is incorporated by reference herein in its entirety. Exemplary TLR2 agonists include mycobacterial cell wall glycolipids, lipoarabinomannan (LAM) and mannosylated phosphatidylinositol (PIIM), MALP-2 and Pam3Cys and synthetic variants thereof. Exemplary TLR4 agonists include lipopolysaccharide or synthetic variants thereof (e.g., MPL and RC529) and lipid A or synthetic variants thereof (e.g., aminoalkyl glucosaminide 4-phosphates). See, e.g., Cluff et al., 2005, Infection and Immunity, p. 3044-3052:73; Lembo et al., 2008, The Journal of Immunology180, 7574-7581; and Evans et al., 2003, Expert Rev Vaccines 2:219-29. Exemplary TLR5 agonists include flagellin or synthetic variants thereof (e.g., A pharmacologically optimized TLR5 agonist with reduced immunogenicity (such as CBLB502) made by deleting portions of flagellin that are non-essential for TLR5 activation).

Additional TLR agonists include Coley's toxin and Bacille Calmette-Guérin (BCG). Coley's toxin is a mixture consisting of killed bacteria of species Streptococcus pyogenes and Serratia marcescens. See Taniguchi et al., 2006, Anticancer Res. 26 (6A): 3997-4002. BCG is prepared from a strain of the attenuated live bovine tuberculosis bacillus, Mycobacterium bovis. See Venkataswamy et al., 2012, Vaccine. 30 (6): 1038-1049.

Cell Based Therapies

Cell-based therapies for the treatment of cancer include administration of immune cells (e.g. T cells, tumor-infiltrating lymphocytes (TILs), Natural Killer cells, and dendritic cells) to a subject. In autologous cell-based therapy, the immune cells are derived from the same subject to which they are administered. In allogeneic cell-based therapy, the immune cells are derived from one subject and administered to a different subject. The immune cells may be activated, for example, by treatment with a cytokine, before administration to the subject. In some embodiments, the immune cells are genetically modified before administration to the subject, for example, as in chimeric antigen receptor (CAR) T cell immunotherapy.

In some embodiments, the cell-based therapy include an adoptive cell transfer (ACT). ACT typically consists of three parts: lympho-depletion, cell administration, and therapy with high doses of IL-2. Types of cells that may be administered in ACT include tumor infiltrating lymphocytes (TILs), T cell receptor (TCR)-transduced T cells, and chimeric antigen receptor (CAR) T cells.

Tumor-infiltrating lymphocytes are immune cells that have been observed in many solid tumors, including breast cancer. They are a population of cells comprising a mixture of cytotoxic T cells and helper T cells, as well as B cells, macrophages, natural killer cells, and dendritic cells. The general procedure for autologous TIL therapy is as follows: (1) a resected tumor is digested into fragments; (2) each fragment is grown in IL-2 and the lymphocytes proliferate destroying the tumor; (3) after a pure population of lymphocytes exists, these lymphocytes are expanded; and (4) after expansion up to 10¹¹ cells, lymphocytes are infused into the patient. See Rosenberg et al., 2015, Science 348(6230):62-68, which is incorporated by reference herein in its entirety.

TCR-transduced T cells are generated via genetic induction of tumor-specific TCRs. This is often done by cloning the particular antigen-specific TCR into a retroviral backbone. Blood is drawn from patients and peripheral blood mononuclear cells (PBMCs) are extracted. PBMCs are stimulated with CD3 in the presence of IL-2 and then transduced with the retrovirus encoding the antigen-specific TCR. These transduced PBMCs are expanded further in vitro and infused back into patients. See Robbins et al., 2015, Clinical Cancer Research 21(5):1019-1027, which is incorporated by reference herein in its entirety.

Chimeric antigen receptors (CARs) are recombinant receptors containing an extracellular antigen recognition domain, a transmembrane domain, and a cytoplasmic signaling domain (such as CD3, CD28, and 4-1BB). CARs possess both antigen-binding and T-cell-activating functions. Therefore, T cells expressing CARs can recognize a wide range of cell surface antigens, including glycolipids, carbohydrates, and proteins, and can attack malignant cells expressing these antigens through the activation of cytoplasmic costimulation. See Pang et al., 2018, Mol Cancer 17: 91, which is incorporated by reference herein in its entirety.

In some embodiments, the cell-based therapy is a Natural Killer (NK) cell-based therapy. NK cells are large, granular lymphocytes that have the ability to kill tumor cells without any prior sensitization or restriction of major histocompatibility complex (MHC) molecule expression. See Uppendahl et al., 2017, Frontiers in Immunology 8: 1825. Adoptive transfer of autologous lymphokine-activated killer (LAK) cells with high-dose IL-2 therapy have been evaluated in human clinical trials. Similar to LAK immunotherapy, cytokine-induced killer (CIK) cells arise from peripheral blood mononuclear cell cultures with stimulation of anti-CD3 mAb, IFN-γ, and IL-2. CIK cells are characterized by a mixed T-NK phenotype (CD3+CD56+) and demonstrate enhanced cytotoxic activity compared to LAK cells against ovarian and cervical cancer. Human clinical trials investigating adoptive transfer of autologous CIK cells following primary debulking surgery and adjuvant carboplatin/paclitaxel chemotherapy have also been conducted. See Liu et al., 2014, J Immunother 37(2): 116-122.

In some embodiments, the cell-based therapy is a dendritic cell-based immunotherapy. Vaccination with dendritic cells (DC)s treated with tumor lysates has been shown to increase therapeutic antitumor immune responses both in vitro and in vivo. See Jung et al., 2018, Translational Oncology 11(3): 686-690. DCs capture and process antigens, migrate into lymphoid organs, express lymphocyte costimulatory molecules, and secrete cytokines that initiate immune responses. They also stimulate immunological effector cells (T cells) that express receptors specific for tumor-associated antigens and reduce the number of immune repressors such as CD4+CD25+Foxp3+ regulatory T (Treg) cells. For example, a DC vaccination strategy for renal cell carcinoma (RCC), which is based on a tumor cell lysate-DC hybrid, showed therapeutic potential in preclinical and clinical trials. See Lim et al., 2007, Cancer Immunol Immunother 56: 1817-1829.

Cytokines

Several cytokines including IL-2, IL-12, IL-15, IL-18, and IL-21 have been used in the treatment of cancer for activation of immune cells such as NK cells and T cells. IL-2 was one of the first cytokines used clinically, with hopes of inducing antitumor immunity. As a single agent at high dose IL-2 induces remissions in some patients with renal cell carcinoma (RCC) and metastatic melanoma. Low dose IL-2 has also been investigated and aimed at selectively ligating the IL-2 αβγ receptor (IL-2Rαβγ) in an effort to reduce toxicity while maintaining biological activity. See Romee et al., 2014, Scientifica, Volume 2014, Article ID 205796, 18 pages, which is incorporated by reference herein in its entirety.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). Recombinant IL-15 has been evaluated for treatment of solid tumors (e.g. melanoma, renal cell carcinoma) and to support NK cells after adoptive transfer in cancer patients. See Romee et al., cited above.

IL-12 is a heterodimeric cytokine composed of p35 and p40 subunits (IL-12α and β chains), originally identified as “NK cell stimulatory factor (NKSF)” based on its ability to enhance NK cell cytotoxicity. Upon encounter with pathogens, IL-12 is released by activated dendritic cells and macrophages and binds to its cognate receptor, which is primarily expressed on activated T and NK cells. Numerous preclinical studies have suggested that IL-12 has antitumor potential. See Romee et al., cited above.

IL-18 is a member of the proinflammatory IL-1 family and, like IL-12, is secreted by activated phagocytes. IL-18 has demonstrated significant antitumor activity in preclinical animal models, and has been evaluated in human clinical trials. See Robertson et al., 2006, Clinical Cancer Research 12: 4265-4273.

IL-21 has been used for antitumor immunotherapy due to its ability to stimulate NK cells and CD8+ T cells. For ex vivo NK cell expansion, membrane bound IL-21 has been expressed in K562 stimulator cells, with effective results. See Denman et al., 2012, PLoS One 7(1)e30264. Recombinant human IL-21 was also shown to increase soluble CD25 and induce expression of perforin and granzyme B on CD8+ cells. IL-21 has been evaluated in several clinical trials for treatment of solid tumors. See Romee et al., cited above.

Cancer Vaccines

Therapeutic cancer vaccines eliminate cancer cells by strengthening a patients' own immune responses to the cancer, particularly CD8+ T cell mediated responses, with the assistance of suitable adjuvants. The therapeutic efficacy of cancer vaccines is dependent on the differential expression of tumor associated antigens (TAAs) by tumor cells relative to normal cells. TAAs derive from cellular proteins and should be mainly or selectively expressed on cancer cells to avoid either immune tolerance or autoimmunity effects. See Circelli et al., 2015, Vaccines 3(3): 544-555. Cancer vaccines include, for example, dendritic cell (DC) based vaccines, peptide/protein vaccines, genetic vaccines, and tumor cell vaccines. See Ye et al., 2018, J Cancer 9(2): 263-268.

Combination Therapies Comprising Agents that Induce Iron-Dependent Cellular Disassembly and Immuno Therapeutics

Methods are provided for the treatment of oncological disorders by administering an agent that induces iron-dependent cellular disassembly in combination with at least one immune checkpoint modulator to a subject. In certain embodiments, the immune checkpoint modulator stimulates the immune response of the subject. For example, in some embodiments, the immune checkpoint modulator stimulates or increases the expression or activity of a stimulatory immune checkpoint (e.g. CD27, CD28, CD40, CD122, OX40, GITR, ICOS, or 4-1BB). In some embodiments, the immune checkpoint modulator inhibits or decreases the expression or activity of an inhibitory immune checkpoint (e.g. A2A4, B7-H3, B7-H4, BTLA, CTLA-4, IDO, KIR, LAG3, PD-1, PD-L1, PD-L2, TIM-3 or VISTA).

In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, CTLA-4, IDO, KIR, LAG3, PD-1, PD-L1, PD-L2, TIM-3 and VISTA. In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, IDO, KIR, LAG3, PD-1, PD-L1, PD-L2, TIM-3 and VISTA. In a particular embodiment, the immune checkpoint modulator targets an immune checkpoint molecule selected from the group consisting of CTLA-4, PD-L1 and PD-1. In a further particular embodiment the immune checkpoint modulator targets an immune checkpoint molecule selected from PD-L1 and PD-1.

In some embodiments, more than one (e.g. 2, 3, 4, 5 or more) immune checkpoint modulator is administered to the subject. Where more than one immune checkpoint modulator is administered, the modulators may each target a stimulatory immune checkpoint molecule, or each target an inhibitory immune checkpoint molecule. In other embodiments, the immune checkpoint modulators include at least one modulator targeting a stimulatory immune checkpoint and at least one immune checkpoint modulator targeting an inhibitory immune checkpoint molecule. In certain embodiments, the immune checkpoint modulator is a binding protein, for example, an antibody. The term “binding protein”, as used herein, refers to a protein or polypeptide that can specifically bind to a target molecule, e.g. an immune checkpoint molecule. In some embodiments the binding protein is an antibody or antigen binding portion thereof, and the target molecule is an immune checkpoint molecule. In some embodiments the binding protein is a protein or polypeptide that specifically binds to a target molecule (e.g., an immune checkpoint molecule). In some embodiments the binding protein is a ligand. In some embodiments, the binding protein is a fusion protein. In some embodiments, the binding protein is a receptor. Examples of binding proteins that may be used in the methods of the invention include, but are not limited to, a humanized antibody, an antibody Fab fragment, a divalent antibody, an antibody drug conjugate, a scFv, a fusion protein, a bivalent antibody, and a tetravalent antibody.

The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In other embodiments, the antibody is a chimeric antibody. Chimeric and humanized antibodies may be prepared by methods well known to those of skill in the art including CDR grafting approaches (see, e.g., U.S. Pat. Nos. 5,843,708; 6,180,370; 5,693,762; 5,585,089; and 5,530,101), chain shuffling strategies (see, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) NATURE 341: 544-546; and WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) SCIENCE 242:423-426; and Huston et al. (1988) PROC. NAT'L. ACAD. SCI. USA 85:5879-5883). Such single chain antibodies are also intended to be encompassed within the term “antigen-binding portion” of an antibody. Other forms of single chain antibodies, such as diabodies are also encompassed. Antigen binding portions can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262(5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “humanized antibody”, as used herein refers to non-human (e.g., murine) antibodies that are chimeric immunoglobulins, immunoglobulin chains, or fragments thereof (such as Fv, Fab, Fab′, F(ab′)2 or other antigen-binding subsequences of antibodies) which contain minimal sequence derived from a non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al. (1986) NATURE 321: 522-525; Reichmann et al. (1988) NATURE 332: 323-329; and Presta (1992) CURR. OP. STRUCT. BIOL. 2: 593-596, each of which is incorporated by reference herein in its entirety.

The term “immunoconjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

A “bivalent antibody” refers to an antibody or antigen-binding fragment thereof that comprises two antigen-binding sites. The two antigen binding sites may bind to the same antigen, or they may each bind to a different antigen, in which case the antibody or antigen-binding fragment is characterized as “bispecific.” A “tetravalent antibody” refers to an antibody or antigen-binding fragment thereof that comprises four antigen-binding sites. In certain embodiments, the tetravalent antibody is bispecific. In certain embodiments, the tetravalent antibody is multispecific, i.e. binding to more than two different antigens.

Fab (fragment antigen binding) antibody fragments are immunoreactive polypeptides comprising monovalent antigen-binding domains of an antibody composed of a polypeptide consisting of a heavy chain variable region (V_{H}) and heavy chain constant region 1 (C_{H1}) portion and a poly peptide consisting of a light chain variable (V_{L}) and light chain constant (C_{L}) portion, in which the C_{L} and C_{H1} portions are bound together, preferably by a disulfide bond between Cys residues.

Immune checkpoint modulator antibodies include, but are not limited to, at least 4 major categories: i) antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, and KIR), ii) antibodies that activate stimulatory pathways directly on T cells or NK cells (e.g., antibodies targeting OX40, GITR, and 4-1BB), iii) antibodies that block a suppressive pathway on immune cells or relies on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, and Ly6G), and iv) antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, and MUC1). Examples of checkpoint inhibitors include, e.g., an inhibitor of CTLA-4, such as ipilimumab or tremelimumab; an inhibitor of the PD-1 pathway such as an anti-PD-1, anti-PD-L1 or anti-PD-L2 antibody. Exemplary anti-PD-1 antibodies are described in WO 2006/121168, WO 2008/156712, WO 2012/145493, WO 2009/014708 and WO 2009/114335. Exemplary anti-PD-L1 antibodies are described in WO 2007/005874, WO 2010/077634 and WO 2011/066389, and exemplary anti-PD-L2 antibodies are described in WO 2004/007679.

In a particular embodiment, the immune checkpoint modulator is a fusion protein, for example, a fusion protein that modulates the activity of an immune checkpoint modulator.

In one embodiment, the immune checkpoint modulator is a therapeutic nucleic acid molecule, for example a nucleic acid that modulates the expression of an immune checkpoint protein or mRNA. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. In certain embodiments, the nucleic acid therapeutic is targeted against a nucleic acid sequence encoding an immune checkpoint protein.

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses include, for example: U.S. Pat. No. 5,898,031 related to chemically modified RNA-containing therapeutic compounds; U.S. Pat. No. 6,107,094 related methods of using these compounds as therapeutic agents; U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality of RNA nucleosides and at least one chemical modification. The entire contents of each of the patents listed in this paragraph are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in WO/2012/037254, and WO 2009/073809, the entire contents of each of which are incorporated herein by reference.

Immune checkpoint modulators may be administered at appropriate dosages to treat the oncological disorder, for example, by using standard dosages. One skilled in the art would be able, by routine experimentation, to determine what an effective, non-toxic amount of an immune checkpoint modulator would be for the purpose of treating oncological disorders. Standard dosages of immune checkpoint modulators are known to a person skilled in the art and may be obtained, for example, from the product insert provided by the manufacturer of the immune checkpoint modulator. Examples of standard dosages of immune checkpoint modulators are provided in Table 3 below. In other embodiments, the immune checkpoint modulator is administered at a dosage that is different (e.g. lower) than the standard dosages of the immune checkpoint modulator used to treat the oncological disorder under the standard of care for treatment for a particular oncological disorder.

TABLE 3
Exemplary Standard Dosages of Immune Checkpoint Modulators

| | Immune | |
|---|---|---|
| | Check- | |
| Immune | point | |
| Checkpoint | Molecule | |
| Modulator | Targeted | Exemplary Standard Dosage |
| Ipilimumab | CTLA-4 | 3 mg/kg administered intravenously over 90 |
| (Yervoy ™) | | minutes every 3 weeks for a total of 4 doses |
| Pembrolizumab | PD-1 | 2 mg/kg administered as an intravenous |
| (Keytruda ™) | | infusion over 30 minutes every 3 weeks until |
| | | disease progression or unacceptable toxicity |
| Atezolizumab | PD-Ll | 1200 mg administered as an intravenous |
| (Tecentriq ™) | | infusion over 60 minutes every 3 weeks |

In certain embodiments, the administered dosage of the immune checkpoint modulator is 5%, 10%, 20%, 30%, 40%, 50%, 60%, 70%, 80%, or 90% lower than the standard dosage of the immune checkpoint modulator for a particular oncological disorder. In certain embodiments, the dosage administered of the immune checkpoint modulator is 95%, 90%, 85%, 80%, 75%, 70%, 65%, 60%, 55%, 50%, 45%, 40%, 35%, 30%, 25%, 20%, 15%, 10% or 5% of the standard dosage of the immune checkpoint modulator for a particular oncological disorder. In one embodiment, where a combination of immune checkpoint modulators are administered, at least one of the immune checkpoint modulators is administered at a dose that is lower than the standard dosage of the immune checkpoint modulator for a particular oncological disorder. In one embodiment, where a combination of immune checkpoint modulators are administered, at least two of the immune checkpoint modulators are administered at a dose that is lower than the standard dosage of the immune checkpoint modulators for a particular oncological disorder. In one embodiment, where a combination of immune checkpoint modulators are administered, at least three of the immune checkpoint modulators are administered at a dose that is lower than the standard dosage of the immune checkpoint modulators for a particular oncological disorder. In one embodiment, where a combination of immune checkpoint modulators are administered, all of the immune checkpoint modulators are administered at a dose that is lower than the standard dosage of the immune checkpoint modulators for a particular oncological disorder.

Co-Administration of an Agent that Induces Iron-Dependent Cellular Disassembly and Immune Checkpoint Modulators

As used herein, the terms “administering in combination”, “co-administering” or “co-administration” refer to administration of the agent that induces iron-dependent cellular disassembly prior to, concurrently or substantially concurrently with, subsequently to, or intermittently with the administration of the immune checkpoint modulator. In certain embodiments, that agent that induces iron-dependent cellular disassembly is administered prior to administration of the immune checkpoint modulator. In certain embodiments, the agent that induce iron-dependent cellular disassembly is administered concurrently with the immune checkpoint modulator. In certain embodiments, the agent that induces iron-dependent cellular disassembly is administered after administration of the immune checkpoint modulator.

The agent that induces iron-dependent cellular disassembly and the immune checkpoint modulator can act additively or synergistically. In one embodiment, the agent that induces iron-dependent cellular disassembly and the immune checkpoint modulator act synergistically. In some embodiments the synergistic effects are in the treatment of the oncological disorder. For example, in one embodiment, the combination of the agent that induces iron-dependent cellular disassembly and the immune checkpoint modulator improves the durability, i.e. extends the duration, of the immune response against the cancer that is targeted by the immune checkpoint modulator. In some embodiments, the agent that induces iron-dependent cellular disassembly and the immune checkpoint modulator act additively.

The combination therapies of the present invention may be utilized for the treatment of oncological disorders. In some embodiments, the combination therapy of the agent that induces iron-dependent cellular disassembly and the immune checkpoint modulator inhibits tumor cell growth. Accordingly, the invention further provides methods of inhibiting tumor cell growth in a subject, comprising administering an agent that induces iron-dependent cellular disassembly and at least one immune checkpoint modulator to the subject, such that tumor cell growth is inhibited. In certain embodiments, treating cancer comprises extending survival or extending time to tumor progression as compared to a control. In some embodiments, the control is a subject that is treated with the immune checkpoint modulator, but is not treated with the agent that induces iron-dependent cellular disassembly. In some embodiments, the control is a subject that is treated with the agent that induces iron-dependent cellular disassembly, but is not treated with the immune checkpoint modulator. In some embodiments, the control is a subject that is not treated with the immune checkpoint modulator or the agent that induces iron-dependent cellular disassembly. In certain embodiments, the subject is a human subject. In preferred embodiments, the subject is identified as having a tumor prior to administration of the first dose of the agent that induces iron-dependent cellular disassembly or the first dose of the immune checkpoint modulator. In certain embodiments, the subject has a tumor at the time of the first administration of the agent that induces iron-dependent cellular disassembly or at the time of first administration of the immune checkpoint modulator.

In certain embodiments, at least 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

It should be noted that more than one immune checkpoint modulator e.g., 2, 3, 4, 5, or more immune checkpoint modulators, may be administered in combination with the agent that induces iron-dependent cellular disassembly.

In one embodiment, administration of the agent that induces iron-dependent cellular disassembly and the immune checkpoint modulator as described herein results in one or more of, reducing tumor size, weight or volume, increasing time to progression, inhibiting tumor growth and/or prolonging the survival time of a subject having an oncological disorder. In certain embodiments, administration of the agent that induces iron-dependent cellular disassembly and the immune checkpoint modulator reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is administered the agent that induces iron-dependent cellular disassembly alone or the immune checkpoint modulator alone. In certain embodiments, administration of the agent that induces iron-dependent cellular disassembly and the immune checkpoint modulator reduces tumor size, weight or volume, increases time to progression, inhibits tumor growth and/or prolongs the survival time of a population of subjects afflicted with an oncological disorder by at least 1%, 2%, 3%, 4%, 5%, 10%, 20%, 30%, 40%, 50%, 60%, 70%, 80%, 90%, 100%, 200%, 300%, 400% or 500% relative to a corresponding population of control subjects afflicted with the oncological disorder that is administered the agent that induces iron-dependent cellular disassembly alone or the immune checkpoint modulator alone. In other embodiments, administration of the agent that induces iron-dependent cellular disassembly and the immune checkpoint modulator stabilizes the oncological disorder in a subject with a progressive oncological disorder prior to treatment.

In certain embodiments, treatment with the agent that induces iron-dependent cellular disassembly and the at least one immune checkpoint modulator is combined with an additional anti-neoplastic agent such as the standard of care for treatment of the particular cancer to be treated, for example by administering a standard dosage of one or more antineoplastic (e.g. chemotherapeutic) agents. The standard of care for a particular cancer type can be determined by one of skill in the art based on, for example, the type and severity of the cancer, the age, weight, gender, and/or medical history of the subject, and the success or failure of prior treatments. In certain embodiments of the invention, the standard of care includes any one of or a combination of surgery, radiation, hormone therapy, antibody therapy, therapy with growth factors, cytokines, and chemotherapy. In one embodiment, the additional anti-neoplastic agent is not an agent that induces iron-dependent cellular disassembly and/or an immune checkpoint modulator.

Additional anti-neoplastic agents suitable for use in the methods disclosed herein include, but are not limited to, chemotherapeutic agents (e.g., alkylating agents, such as Altretamine, Busulfan, Carboplatin, Carmustine, Chlorambucil, Cisplatin, Cyclophosphamide, Dacarbazine, Lomustine, Melphalan, Oxaliplatin, Temozolomide, Thiotepa; antimetabolites, such as 5-fluorouracil (5-FU), 6-mercaptopurine (6-MP); Capecitabine (Xeloda®), Cytarabine (Ara-C®), Floxuridine, Fludarabine, Gemcitabine (Gemzar®), Hydroxyurea, Methotrexate, Pemetrexed (Alimta®); anti-tumor antibiotics such as anthracyclines (e.g., Daunorubicin, Doxorubicin (Adriamycin®), Epirubicin, Idarubicin), Actinomycin-D, Bleomycin, Mitomycin-C, Mitoxantrone (also acts as a topoisomerase II inhibitor); topoisomerase inhibitors, such as Topotecan, Irinotecan (CPT-11), Etoposide (VP-16), Teniposide, Mitoxantrone (also acts as an anti-tumor antibiotic); mitotic inhibitors such as Docetaxel, Estramustine, Ixabepilone, Paclitaxel, Vinblastine, Vincristine, Vinorelbine; corticosteroids such as Prednisone, Methylprednisolone (Solumedrol®), Dexamethasone (Decadron®); enzymes such as L-asparaginase, and bortezomib (Velcade®)). Anti-neoplastic agents also include biologic anti-cancer agents, e.g., anti-TNF antibodies, e.g., adalimumab or infliximab; anti-CD20 antibodies, such as rituximab, anti-VEGF antibodies, such as bevacizumab; anti-HER2 antibodies, such as trastuzumab; anti-RSV, such as palivizumab. Cancers for treatment using the methods of the invention include, for example, all types of cancer or neoplasm or malignant tumors found in mammals, including, but not limited to: sarcomas, melanomas, carcinomas, leukemias, and lymphomas.

The term “sarcoma” generally refers to a tumor which is made up of a substance like the embryonic connective tissue and is generally composed of closely packed cells embedded in a fibrillar or homogeneous substance. Examples of sarcomas which can be treated with the methods of the invention include, for example, a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, uterine sarcoma, myxoid liposarcoma, leiomyosarcoma, spindle cell sarcoma, desmoplastic sarcoma, and telangiectaltic sarcoma.

The term “melanoma” is taken to mean a tumor arising from the melanocytic system of the skin and other organs. Melanomas which can be treated with the methods of the invention include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with the methods of the invention, as described herein, include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, colon adenocarcinoma of colon, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, merkel cell carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, cervical squamous cell carcinoma, tonsil squamous cell carcinoma, and carcinoma villosum. In a particular embodiment, the cancer is renal cell carcinoma.

The term “leukemia” refers to a type of cancer of the blood or bone marrow characterized by an abnormal increase of immature white blood cells called “blasts”. Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia. In certain embodiments, leukemias include acute leukemias. In certain embodiments, leukemias include chronic leukemias.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

In some embodiments, the compositions are used for treatment of various types of solid tumors, for example breast cancer (e.g. triple negative breast cancer), bladder cancer, genitourinary tract cancer, colon cancer, rectal cancer, endometrial cancer, kidney (renal cell) cancer, pancreatic cancer, prostate cancer, thyroid cancer (e.g. papillary thyroid cancer), skin cancer, bone cancer, brain cancer, cervical cancer, liver cancer, stomach cancer, mouth and oral cancers, esophageal cancer, adenoid cystic cancer, neuroblastoma, testicular cancer, uterine cancer, thyroid cancer, head and neck cancer, kidney cancer, lung cancer (e.g. small cell lung cancer, non-small cell lung cancer), mesothelioma, ovarian cancer, sarcoma, stomach cancer, uterine cancer, cervical cancer, medulloblastoma, and vulvar cancer. In certain embodiments, skin cancer includes melanoma, squamous cell carcinoma, and cutaneous T-cell lymphoma (CTCL).

Additional cancers which can be treated with the compositions of the invention include, for example, multiple myeloma, primary thrombocytosis, primary macroglobulinemia, malignant pancreatic insulanoma, malignant carcinoid, malignant hypercalcemia, endometrial cancer, adrenal cortical cancer, and malignant fibrous histiocytoma.

In some embodiments, the combination therapies described herein may be administered to a subject that has previously failed treatment for a cancer with another anti-neoplastic (e.g. chemotherapeutic) regimen. A “subject who has failed an anti-neoplastic regimen” is a subject with cancer that does not respond, or ceases to respond to treatment with a anti-neoplastic regimen per RECIST 1.1 criteria, i.e., does not achieve a complete response, partial response, or stable disease in the target lesion; or does not achieve complete response or non-CR/non-PD of non-target lesions, either during or after completion of the anti-neoplastic regimen, either alone or in conjunction with surgery and/or radiation therapy which, when possible, are often clinically indicated in conjunction with anti-neoplastic therapy. The RECIST 1.1 criteria are described, for example, in Eisenhauer et al., 2009, Eur. J. Cancer 45:228-24 (which is incorporated herein by reference in its entirety), and discussed in greater detail below. A failed anti-neoplastic regimen results in, e.g., tumor growth, increased tumor burden, and/or tumor metastasis. A failed anti-neoplastic regimen as used herein includes a treatment regimen that was terminated due to a dose limiting toxicity, e.g., a grade III or a grade IV toxicity that cannot be resolved to allow continuation or resumption of treatment with the anti-neoplastic agent or regimen that caused the toxicity. In one embodiment, the subject has failed treatment with a anti-neoplastic regimen comprising administration of one or more anti-angiogenic agents.

A failed anti-neoplastic regimen includes a treatment regimen that does not result in at least stable disease for all target and non-target lesions for an extended period, e.g., at least 1 month, at least 2 months, at least 3 months, at least 4 months, at least 5 months, at least 6 months, at least 12 months, at least 18 months, or any time period less than a clinically defined cure. A failed anti-neoplastic regimen includes a treatment regimen that results in progressive disease of at least one target lesion during treatment with the anti-neoplastic agent, or results in progressive disease less than 2 weeks, less than 1 month, less than two months, less than 3 months, less than 4 months, less than 5 months, less than 6 months, less than 12 months, or less than 18 months after the conclusion of the treatment regimen, or less than any time period less than a clinically defined cure.

A failed anti-neoplastic regimen does not include a treatment regimen wherein the subject treated for a cancer achieves a clinically defined cure, e.g., 5 years of complete response after the end of the treatment regimen, and wherein the subject is subsequently diagnosed with a distinct cancer, e.g., more than 5 years, more than 6 years, more than 7 years, more than 8 years, more than 9 years, more than 10 years, more than 11 years, more than 12 years, more than 13 years, more than 14 years, or more than 15 years after the end of the treatment regimen.

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al., 2009, Eur. J. Cancer 45:228-24, which is incorporated herein by reference. Response criteria for target lesions include:

Complete Response (CR): Disappearance of all target lesions. Any pathological lymph nodes (whether target or non-target) must have a reduction in short axis to <10 mm.

Partial Response (PR): At least a 30% decrease in the sum of diameters of target lesion, taking as a reference the baseline sum diameters.

Progressive Diseases (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)

Stable Disease (SD): Neither sufficient shrinkage to qualify for PR nor sufficient increase to qualify for PD, taking as a reference the smallest sum diameters while on study.

RECIST 1.1 criteria also consider non-target lesions which are defined as lesions that may be measureable, but need not be measured, and should only be assessed qualitatively at the desired time points. Response criteria for non-target lesions include:

Complete Response (CR): Disappearance of all non-target lesions and normalization of tumor marker levels. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

In some embodiments, the combination therapies described herein may be administered to a subject having a refractory cancer. A “refractory cancer” is a malignancy for which surgery is ineffective, which is either initially unresponsive to chemo- or radiation therapy, or which becomes unresponsive to chemo- or radiation therapy over time.

### V. Pharmaceutical Compositions and Modes of Administration

The pharmaceutical compositions described herein may be administered to a subject in any suitable formulation. These include, for example, liquid, semi-solid, and solid dosage forms, The preferred form depends on the intended mode of administration and therapeutic application.

In certain embodiments the composition is suitable for oral administration. In certain embodiments, the formulation is suitable for parenteral administration, including topical administration and intravenous, intraperitoneal, intramuscular, and subcutaneous, injections. In a particular embodiment, the composition is suitable for intravenous administration. Pharmaceutical compositions for parenteral administration include aqueous solutions of the active compounds in water-soluble form. For intravenous administration, the formulation may be an aqueous solution. The aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. The aqueous solution may contain substances which increase the viscosity of the solution, such as sodium carboxymethyl cellulose, sorbitol, or dextran. In some embodiments, the formulation includes a phosphate buffer saline solution which contains sodium phosphate dibasic, potassium phosphate monobasic, potassium chloride, sodium chloride and water for injection.

Formulations suitable for topical administration include liquid or semi-liquid preparations suitable for penetration through the skin, such as liniments, lotions, creams, ointments or pastes, and drops suitable for administration to the eye, ear, or nose. Formulations suitable for oral administration include preparations containing an inert diluent or an assimilable edible carrier. The formulation for oral administration may be enclosed in hard or soft shell gelatin capsule, or it may be compressed into tablets, or it may be incorporated directly with the food of the diet. When the dosage unit form is a capsule, it may contain, in addition to materials of the above type, a liquid carrier. Various other materials may be present as coatings or to otherwise modify the physical form of the dosage unit. Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically.

As will be readily apparent to one skilled in the art, the useful in vivo dosage to be administered and the particular mode of administration will vary depending upon the age, body weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, animal models, and in vitro studies.

In certain embodiments, the composition is delivered orally. In certain embodiments, the composition is administered parenterally. In certain embodiments, the compositions is delivered by injection or infusion. In certain embodiments, the composition is delivered topically including transmucosally. In certain embodiments, the composition is delivered by inhalation. In one embodiment, the compositions provided herein may be administered by injecting directly to a tumor. In some embodiments, the compositions may be administered by intravenous injection or intravenous infusion. In certain embodiments, administration is systemic. In certain embodiments, administration is local.

### VI. Methods for Identification of Immunostimulating Agents that Induce Iron-Dependent Cellular Disassembly

In addition to the agents that induce iron-dependent cellular disassembly known in the art and described herein, the disclosure further relates to methods for identifying other compounds that induce iron-dependent cellular disassembly and stimulate immune activity.

For example, in certain aspects, the disclosure relates to a method of screening for an immunostimulatory agent, the method comprising:

(a) providing a plurality of test agents (e.g., a library of test agents);

(b) evaluating each of the plurality of test agents for the ability to induce iron-dependent cellular disassembly (e.g., ferroptosis);

(c) selecting as a candidate immunostimulatory agent a test agent that increases iron-dependent cellular disassembly (e.g., ferroptosis); and

(d) evaluating the candidate immunostimulatory agent for the ability to increase an immune response.

In some embodiments, evaluating the test agents for the ability to induce iron-dependent cellular disassembly (e.g., ferroptosis) comprises contacting cells or tissue with each of the plurality of test agents.

Several methods are known in the art and may be employed for identifying cells undergoing iron-dependent cellular disassembly (e.g., ferroptosis) and distinguishing from other types of cellular disassembly and/or cell death through detection of particular markers. (See, for example, Stockwell et al., 2017, Cell 171: 273-285, incorporated by reference herein in its entirety). For example, because iron-dependent cellular disassembly may result from lethal lipid peroxidation, measuring lipid peroxidation provides one method of identifying cells undergoing iron-dependent cellular disassembly. C11-BODIPY and Liperfluo are lipophilic ROS sensors that provide a rapid, indirect means to detect lipid ROS (Dixon et al., 2012, Cell 149: 1060-1072). Liquid chromatography (LC)/tandem mass spectrometry (MS) analysis can also be used to detect specific oxidized lipids directly (Friedmann Angeli et al., 2014, Nat. Cell Biol. 16: 1180-1191; Kagan et al., 2017, Nat. Chem. Biol. 13: 81-90). Isoprostanes and malondialdehyde (MDA) may also be used to measure lipid peroxidation (Milne et al., 2007, Nat. Protoc. 2: 221-226; Wang et al., 2017, Hepatology 66(2): 449-465). Kits for measuring MDA are commercially available (Beyotime, Haimen, China).

Other useful assays for studying iron-dependent cellular disassembly include measuring iron abundance and GPX4 activity. Iron abundance can be measured using inductively coupled plasma-MS or calcein AM quenching, as well as other specific iron probes (Hirayama and Nagasawa, 2017, J. Clin. Biochem. Nutr. 60: 39-48; Spangler et al., 2016, Nat. Chem. Biol. 12: 680-685), while GPX4 activity can be detected using phosphatidylcholine hydroperoxide reduction in cell lysates using LC-MS (Yang et al., 2014, Cell 156: 317-331). In addition, iron-dependent cellular disassembly may be evaluated by measuring glutathione (GSH) content. GSH may be measured, for example, by using the commercially available GSH-Glo Glutathione Assay (Promega, Madison, Wis.).

Iron-dependent cellular disassembly may also be evaluated by measuring the expression of one or more marker proteins. Suitable marker proteins include, but are not limited to, glutathione peroxidase 4 (GPX4), prostaglandin-endoperoxide synthase 2 (PTGS2), and cyclooxygenase-2 (COX-2). The level of expression of the marker protein or a nucleic acid encoding the marker protein may be determined using suitable techniques known in the art including, but not limited to polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, Western blot analysis, in situ hybridization, array analysis, deoxyribonucleic acid sequencing, restriction fragment length polymorphism analysis, and combinations or sub-combinations thereof.

In some embodiments, evaluating the test agents for the ability to induce iron-dependent cellular disassembly comprises measuring the level or activity of a ferroptosis marker, e.g., a marker selected from the group consisting of lipid peroxidation, reactive oxygen species (ROS), isoprostanes, malondialdehyde (MDA), iron, glutathione peroxidase 4 (GPX4), prostaglandin-endoperoxide synthase 2 (PTGS2), cyclooxygenase-2 (COX-2) and glutathione (GSH), in the cells or tissue contacted with the test agent.

In some embodiments, evaluating the test agents for the ability to induce iron-dependent cellular disassembly comprises comparing the level or activity of the marker in the cells or tissue contacted with the test agent to the level or activity of the marker in a control cell or tissue that has not been contacted with the test agent.

In one embodiment, an increase in the level or activity of a marker selected from the group consisting of lipid peroxidation, isoprostanes, reactive oxygen species (ROS), iron, PTGS2 and COX-2, or a decrease in the level or activity of a marker selected from the group consisting of GPX4, MDA and GSH indicates that the test agent is an agent that induces iron-dependent cellular disassembly.

In one embodiment, evaluating the test agents for the ability to induce iron-dependent cellular disassembly comprises measuring lipid peroxides in the cells or tissue contacted with the test agent.

In one embodiment, an increase in the level of lipid peroxides in the cells or tissue contacted with the test agent indicates that the test agent is an agent that induces iron-dependent cellular disassembly.

In one embodiment, evaluating the test agents for the ability to induce iron-dependent cellular disassembly further includes evaluating whether one or more activities (e.g., modulation of a ferroptosis marker, such as lipid peroxidation, and/or immunostimulatory activity) of the test agent is inhibited by a known ferroptosis inhibitor (e.g., ferrostatin, B-Mercaptoethanol or an iron chelator).

In one embodiment, evaluating the candidate immunostimulatory agent for the ability to increase an immune response comprises evaluating the test agent that induces iron-dependent cellular disassembly for immunostimulatory activity. Any of the methods described herein for evaluating immune response may be used for evaluating candidate immunostimulatory agents.

In one embodiment, evaluating the candidate immunostimulatory agent comprises culturing an immune cell together with cells contacted with the selected candidate immunostimulatory agent or exposing an immune cell to postcellular signaling factors produced by cells contacted with the selected candidate immunostimulatory agent and measuring the level or activity of NFκB, IRF or STING in the immune cell.

In one embodiment, the immune cell is a THP-1 cell. For example, NFKB and IRF activity may be measured in commercially available THP1-Dual cells (InvivoGen, San Diego, Calif.). THP1-Dual cells are human monocyte cells that induce reporter proteins upon activation of either NFKB or IRF pathways. The THP-1 cells may be cultured with cells contacted with the selected candidate immunostimulatory agent or exposed to postcellular signaling factors produced by cells contacted with the selected candidate immunostimulatory agent and then mixed with either 200 μl QuantiBlue (InvivoGen, San Diego, Calif.) or 50 μl QuantiLuc for detection of NFKB and IRF activity. NFKB and IRF activity may be quantified by measuring absorbance or luminescence on a Molecular Devices plate reader.

In one embodiment, evaluating the candidate immunostimulatory agent comprises culturing T cells together with cells contacted with the selected candidate immunostimulatory agent or exposing T cells to postcellular signaling factors produced by cells contacted with the selected candidate immunostimulatory agent and measuring the activation and proliferation of the T cells.

In one embodiment, the immune cell is a macrophage. For example, NFKB and IRF activity may be measured in commercially available Raw-Dual™ and J774-Dual™ macrophage cells (InvivoGen, San Diego, Calif.). Raw-Dual™ and J774-Dual™ cells are mouse macrophage cell lines that induce reporter proteins upon activation of either NFKB or IRF pathways. The macrophage cells may be cultured with cells contacted with the selected candidate immunostimulatory agent or exposed to postcellular signaling factors produced by cells contacted with the selected candidate immunostimulatory agent and then mixed with either 200 μl QuantiBlue (InvivoGen, San Diego, Calif.) or 50 μl QuantiLuc for detection of NFKB and IRF activity. NFKB and IRF activity may be quantified by measuring absorbance or luminescence on a Molecular Devices plate reader.

In one embodiment, the immune cell is a Dendritic Cell. For example, co-stimulatory markers (e.g. CD80, CD86) or markers of enhanced antigen presentation (e.g. MHCII) can be measured in dendritic cells by flow cytometry. The dendritic cells may be cultured with cells contacted with the selected candidate immunostimulatory agent or exposed to compounds produced by cells contacted with the selected candidate immunostimulatory agent and then stained with antibodies specific to cell surface markers indicative of activation status. Subsequently, the expression level of these markers is determined by flow cytometry.

Candidate immunostimulatory agents may also be evaluated by measuring pro-immune cytokine levels in macrophages and/or dendritic cells. For example, in some embodiments, evaluating candidate immunostimulatory agents comprises culturing macrophage cells and/or dendritic cells with cells contacted with the selected candidate immunostimulatory agent or contacting macrophage cells and/or dendritic cells with postcellular signaling factors produced by cells contacted with the selected candidate immunostimulatory agent and measuring levels of pro-immune cytokines (e.g. IFN-α, IL-1, IL-12, IL-18, IL-2, IL-15, IL-4, IL-6, TNF-α, IL-17 and GMCSF). Pro-immune cytokine levels may be determined by methods known in the art, such as ELISA.

### VII. Methods for Identification of Postcellular Immunostimulatory Agents Produced by Iron-Dependent Cellular Disassembly

Applicants have shown that treatment of cells with agents that induce iron-dependent cellular disassembly results in the production and release of postcellular signaling factors that increase immune activity. Accordingly, agents that induce iron-dependent cellular disassembly and increase immune activity may be used in the treatment of disorders that may benefit from increased immune activity, such as cancer and infections. In an alternative approach, the postcellular signaling factors produced by the disassembling cell may be isolated and screened for immune activity. In this way, postcellular signaling factors or “effectors” that increase immune activity may be identified for use in the treatment of disorders.

For example, in certain aspects, the disclosure relates to a method of identifying an immunostimulatory agent, the method comprising:

(a) contacting a cell with an agent that induces iron-dependent cellular disassembly in an amount sufficient to induce iron-dependent cellular disassembly in the cell;
(b) isolating one or more postcellular signaling factors produced by the cell after contact with the agent that induces iron-dependent cellular disassembly; and
(c) assaying the one or more postcellular signaling factors for the ability to modulate, e.g., increase or induce, immune response.

The one or more postcellular signaling factors produced by the cell may be isolated, for example, by separating the cell from the medium in which it is grown (e.g. by centrifugation) and subjecting this conditioned medium to further analysis. For example, in some embodiments, the conditioned medium is extracted with organic solvent followed by HPLC fractionation. In other embodiments, the conditioned medium is subjected to size exclusion chromatography and different fractions are collected. For example, conditioned medium may be applied to a size exclusion column and fractionated on FPLC.

The ability of the postcellular signaling factors to modulate immune response may be assayed by contacting the postcellular signaling factors with an immune cell and evaluating immune activity. Any of the methods described herein for measuring immune response such as measuring the level or activity of NFkB, IRF and/or STING, the level or activity of macrophages, the level or activity of monocytes, the level or activity of dendritic cells, the level or activity of CD4+, CD8+ or CD3+ cells, the level or activity of T cells, and the level or activity of a pro-immune cytokine may be used to measure the ability of the postcellular signaling factors to modulate immune response. For example, in some embodiments, collected fractions containing the postcellular signaling factors are applied to THP-1 Dual cells and NFKB and/or IRF1 reporter activity is assessed. Positive hit fractions are confirmed by their ability to induce NFKB or IRF activity in THP1 Dual cells. Positive hit fractions may be further characterized by mass spectrometry (large molecules) or NMR (small molecules) to identify particular compounds with immune activity. The immune activity of the individual compounds or species may be tested by the addition of synthetic or recombinant forms of such compounds or species to THP1 Dual Cells followed by measurement of NFKB or IRF activity, as described above.

The immune activity of the postcellular signaling factors may be determined by applying the postcellular signaling factors to macrophages, monocytes, dendritic cells, CD4+, CD8+ or CD3+ cells, and/or T cells and measuring the level or activity of the cells. For example, in one embodiment, the assaying comprises treating an immune cell with the one or more postcellular signaling factors and measuring the level or activity of NFKB activity in the immune cell. In one embodiment, the assaying comprises treating T cells with the one or more postcellular signaling factors and measuring the activation or proliferation of the T cells. In one embodiment, the assaying comprises contacting an immune cell with the one or more postcellular signaling factors and measuring the level or activity of NFκB, IRF or STING in the immune cell. In one embodiment, the immune cell is a THP-1 cell.

The immunostimulatory activity of the postcellular signaling factors may also be evaluated in animal models, e.g. an animal cancer model. For example, in some embodiments, a postcellular signaling factor is administered to an animal and an immune response is measured in the animal, for example, by measuring changes in the level or activity of NFkB, IRF and/or STING, the level or activity of macrophages, the level or activity of monocytes, the level or activity of dendritic cells, the level or activity of CD4+, CD8+ or CD3+ cells, the level or activity of T cells, and the level or activity of a pro-immune cytokine after administration of the postcellular signaling factor.

In one embodiment, the method further comprises selecting a postcellular signaling factor that stimulates immune response.

In one embodiment, the method further comprises detecting a marker of iron-dependent cellular disassembly (e.g., ferroptosis) in the cell.

Postcellular signaling factors that are produced at higher levels in iron-dependent cellular disassembly (e.g., ferroptosis) relative to cells that are not undergoing cellular disassembly may be identified by comparing levels of postcellular signaling factors in treated and untreated cells. For example, in one embodiment, the method further comprises:

i) measuring the level of the one or more postcellular signaling factors produced by the cell after contact with the agent that induces iron-dependent cellular disassembly;

ii) comparing the level of the one or more postcellular signaling factors produced by the cell after contact with the agent that induces iron-dependent cellular disassembly to the level of the one or more postcellular signaling factors in a control cell that is not treated with the agent that induces iron-dependent cellular disassembly; and

iii) selecting postcellular signaling factors that exhibit increased levels in the cell contacted with the agent that induces iron-dependent cellular disassembly relative to the control cell to generate the one or more postcellular signaling factors for assaying in step (c).

Postcellular signaling factors that are specific to iron-dependent cellular disassembly (e.g., ferroptosis) or produced at higher levels in iron-dependent cellular disassembly (e.g., ferroptosis) relative to other cell death processes may also be identified by comparing postcellular signaling factor levels in cells undergoing different cell death processes.

For example, in one embodiment of the methods described above, the control cell is treated with an agent that induces cellular disassembly that is not iron-dependent cellular disassembly, for example a cell death process that is not ferroptosis, e.g. apoptosis, necroptosis or pyroptosis.

### EXAMPLES

### Example 1: Activation/Stimulation of Human Monocytes by HT1080 Fibrosarcoma Cells Treated with Erastin

Agent/Therapeutic Design:

HT1080 fibrosarcoma cells were treated with either control (DMSO) or various doses of Erastin, piperazine erastin (PE), or imidazole ketoerastin (IKE) for 24 hours prior to co-culture with THP1-Dual cells for an additional 24 hours. Erastin was purchased from Selleckchem (Houston, Tex.) and dissolved in DMSO. Subsequently, the THP1 supernatants were assessed for nuclear factor kappa-light-chain-enhancer of activated B cells (NFKB) or interferon regulatory factor (IRF) reporter activity.

Materials/Methods:

HT1080 cells were acquired from ATCC and THP1-Dual cells were acquired from InvivoGen (San Diego, Calif.). THP1-Dual cells are human monocyte cells that induce reporter proteins upon activation of either NFKB or IRF pathways. Both cell types were cultured in 96-well plates for the duration of the assay. HT1080 cells were cultured in DMEM with 10% FBS, and THP1-Dual cells were cultured in RPMI with 10% FBS. 7,500 HT1080 cells were plated 24 hours prior to dosing with Erastin, PE, or IKE, with a final DMSO concentration of 0.5%. 24 hours post-treatment, THP1-Dual cells (25,000 cells/well) were added to the HT1080 cells. 24 hours later, 30 μl of supernatant was mixed with either 200 μl QuantiBlue (InvivoGen, San Diego, Calif.) (for NFKB reporter activity) or 50 μl QuantiLuc (for IRF reporter activity) and absorbance or luminescence was recorded on a Molecular Devices plate reader.

Conclusion:

As shown in FIG. 1A, Erastin treatment negatively affected the viability of HT1080 cells.

FIG. 1B shows that HT1080 cells treated with Erastin, but not the vehicle control DMSO, elicited NFKB signaling in THP1 monocytes. The erastin analogs PE and IKE also negatively affected the viability of HT1080 cells (FIG. 1C) and elicited NFKB signaling in THP1 monocytes (FIG. 1D).

### Example 2: Activation/Stimulation of Human Monocytes by PANC1 Pancreatic Cancer Cells Treated with Erastin

Agent/Therapeutic Design:

PANC1 pancreatic cancer cells were treated with either control (DMSO) or various doses of Erastin, for 24 hours prior to co-culture with THP1-Dual cells for an additional 24 hours. Erastin was purchased from Selleckchem (Houston, Tex.) and dissolved in DMSO. Subsequently, the THP1 supernatants were assessed for NFKB or IRF reporter activity.

Materials/Methods:

PANC1 cells were acquired from ATCC and THP1-Dual cells were acquired from InvivoGen (San Diego, Calif.). Both cell types were cultured in 96-well plates for the duration of the assay. 7,500 PANC-1 cells were plated 24 hours prior to dosing with Erastin, with a final DMSO concentration of 0.5%. 24 hours post-treatment, THP1-Dual cells (25,000 cells/well) were added to the PANC-1 cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

Conclusion:

As shown in FIG. 2A, Erastin treatment negatively affected the viability of PANC1 cells. FIG. 1B shows that PANC1 cells treated with Erastin, but not the vehicle control DMSO, elicited NFKB signaling in THP1 monocytes.

### Example 3: Activation/Stimulation of Human Monocytes by Caki-1 Renal Cell Carcinoma Cells Treated with Erastin

Agent/Therapeutic Design:

Caki-1 renal cell carcinoma cells were treated with either control (DMSO) or various doses of Erastin, for 24 hours prior to co-culture with THP1-Dual cells for an additional 24 hours. Erastin was purchased from Selleckchem (Houston, Tex.) and dissolved in DMSO. Subsequently, the THP1 supernatants were assessed for NFKB or IRF reporter activity.

Materials/Methods:

Caki-1 cells were acquired from ATCC and THP1-Dual cells acquired from InvivoGen (San Diego, Calif.). Both cell types were cultured in 96-well plates for the duration of the assay. 7,500 Caki-1 cells were plated 24 hours prior to dosing with Erastin, with a final DMSO concentration of 0.5%. 24 hours post-treatment, THP1-Dual cells (25,000 cells/well) were added to the Caki-1 cells. 24 hours later, 30 μl of supernatant was mixed with either 200 μl QuantiBlue (InvivoGen) or 50 μl QuantiLuc and absorbance or luminescence was recorded on a Molecular Devices plate reader.

Conclusion:

As shown in FIG. 3A, Erastin treatment negatively affected the viability of Caki-1 cells. FIG. 3B shows that Caki-1 cells treated with Erastin, but not the vehicle control DMSO, elicited NFKB signaling in THP1 monocytes.

### Example 4: Activation/Stimulation of Human Monocytes by Caki-1 Renal Cell Carcinoma Cells Treated with RSL3

Agent/Therapeutic Design:

Caki-1 renal cell carcinoma cells were treated with either control (DMSO) or various doses of RSL3, for 24 hours prior to co-culture with THP1-Dual cells for an additional 24 hours. RSL3 was purchased from Selleckchem and dissolved in DMSO. Subsequently, the THP1 supernatants were assessed for NFKB or IRF reporter activity.

Materials/Methods:

Caki-1 cells were acquired from ATCC and THP1-Dual cells acquired from InvivoGen (San Diego, Calif.). Both cell types were cultured in 96-well plates for the duration of the assay. 7,500 Caki-1 cells were plated 24 hours prior to dosing with RSL3, with a final DMSO concentration of 0.5%. 24 hours post-treatment, THP1-Dual cells (25,000 cells/well) were added to the Caki-1 cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

Conclusion:

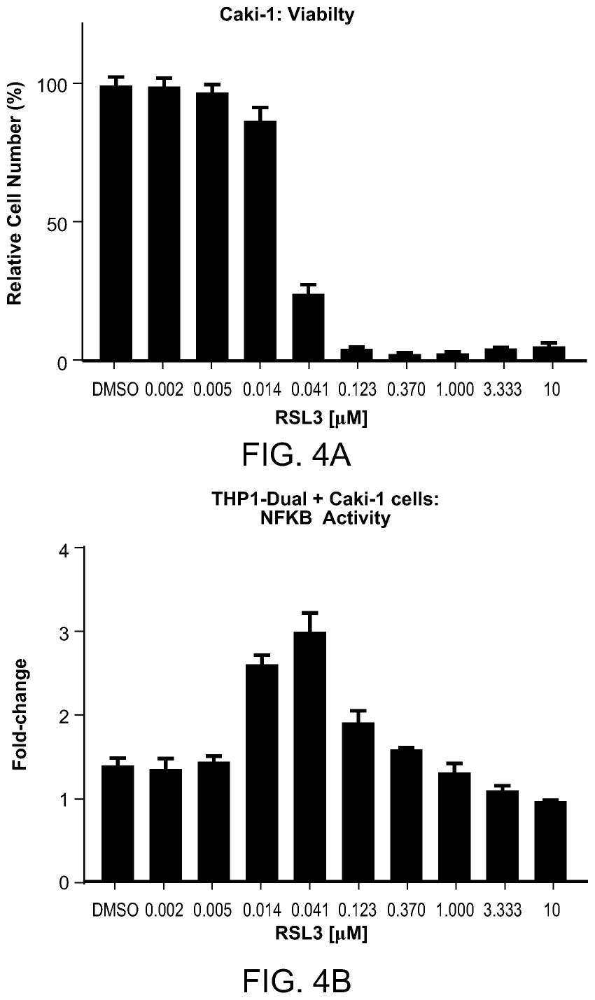

As shown in FIG. 4A, RSL3 treatment negatively affected the viability of Caki-1 cells. FIG. 4B shows that Caki-1 cells treated with RSL3, but not the vehicle control DMSO, elicited NFKB signaling in THP1 monocytes.

### Example 5: Activation/Stimulation of Human Monocytes by Jurkat T Cell Leukemia Cells Treated with RSL3

Agent/Therapeutic Design:

Jurkat T cell leukemia cells were treated with either control (DMSO) or various doses of RSL3, for 24 hours prior to co-culture with THP1-Dual cells for an additional 24 hours. RSL3 was purchased from Selleckchem (Houston, Tex.) and dissolved in DMSO. Subsequently, the THP1 supernatants were assessed for NFKB or IRF reporter activity.

Materials/Methods:

Jurkat cells were acquired from ATCC and THP1-Dual cells acquired from InvivoGen (San Diego, Calif.). Both cell types were cultured in 96-well plates for the duration of the assay. 100,000 Jurkat cells were plated 24 hours prior to dosing with RSL3, with a final DMSO concentration of 0.5%. 24 hours post-treatment, THP1-Dual cells (25,000 cells/well) were added to the Jurkat cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

Conclusion:

As shown in FIG. 5A, RSL3 treatment negatively affected the viability of Jurkat T cell leukemia cells. FIG. 5B shows that Jurkat cells treated with RSL3, but not the vehicle control DMSO, elicited NFKB signaling in THP1 monocytes.

### Example 6: Activation/Stimulation of Human Monocytes by A20 B-Cell Leukemia Cells Treated with RSL3

Agent/Therapeutic Design:

A20 B-cell leukemia cells were treated with either control (DMSO) or various doses of RSL3, for 24 hours prior to co-culture with THP1-Dual cells for an additional 24 hours. RSL3 was purchased from Selleckchem (Houston, Tex.) and dissolved in DMSO. Subsequently, the THP1 supernatants were assessed for NFKB or IRF reporter activity.

Materials/Methods:

A20 cells were acquired from ATCC and THP1-Dual cells acquired from InvivoGen (San Diego, Calif.). Both cell types were cultured in 96-well plates for the duration of the assay. 50,000 A20 cells were plated 24 hours prior to dosing with RSL3, with a final DMSO concentration of 0.5%. 24 hours post-treatment, THP1-Dual cells (25,000 cells/well) were added to the A20 cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

Conclusion:

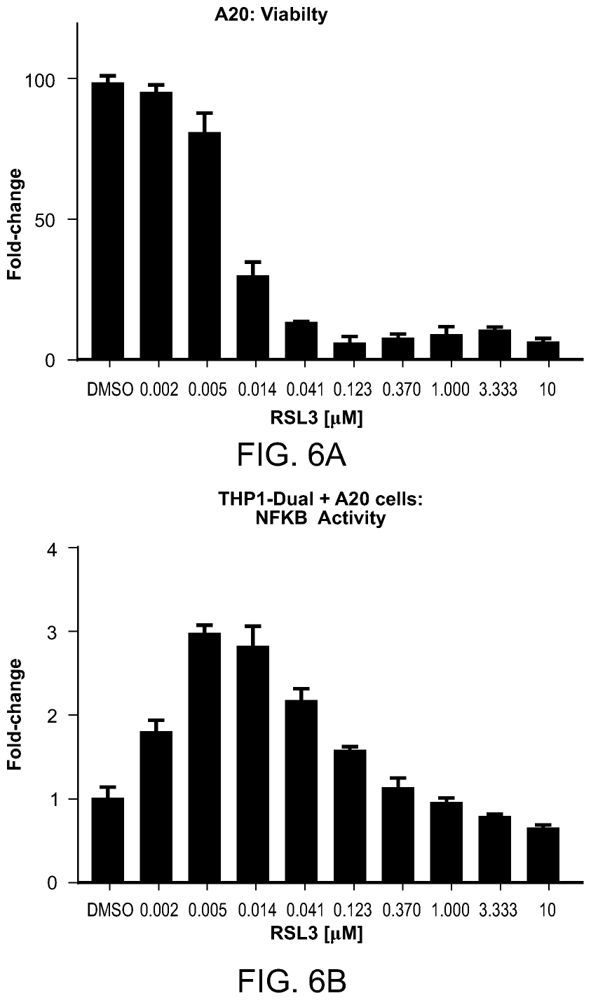

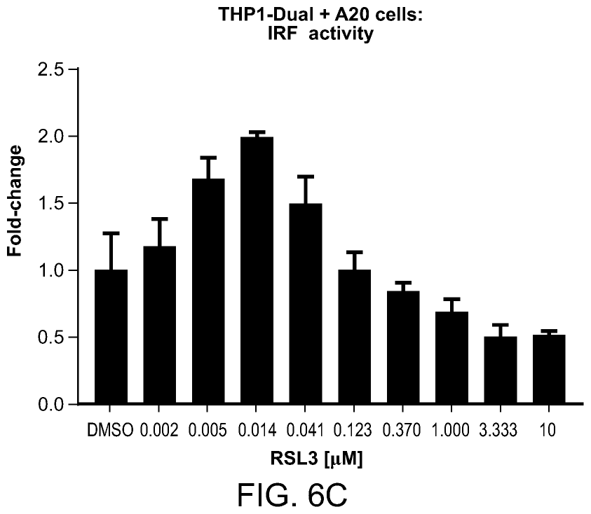

As shown in FIG. 6A, RSL3-treatment negatively affected the viability of A20 B cell leukemia cells. A20 cells treated with RSL3, but not the vehicle control DMSO, elicited NFKB (FIG. 6B) and IRF (FIG. 6C) signaling in THP1 monocytes.

### Example 7: Specificity of Pro-Inflammatory Signaling Elicited by HT1080 Fibrosarcoma Cells Treated with Erastin

Agent/Therapeutic Design:

HT1080 fibrosarcoma cells are treated with either control (DMSO) or various doses of Erastin (e.g. 0.098, 0.195, 0.391, 0.781, 1.563, 3.125, 6.25, 12.5 and 25 μM) in the presence or absence of 1 μM Ferrostatin for 24 hours prior to co-culture with THP1-Dual cells for an additional 24 hours. Ferrostatin is purchased from Selleckchem (Houston, Tex.) and dissolved in DMSO. Subsequently, the THP1 supernatant is assessed for NFKB or IRF reporter activity. HT1080 cells are acquired from ATCC and THP1-Dual cells acquired from InvivoGen (San Diego, Calif.). Both cell types are cultured in 96-well plates for the duration of the assay. The specificity of induction of NFKB signaling elicited by Erastin-treated HT1080 cells is assessed by its reversal by concomitant ferrostatin treatment of HT1080 cells.

### Example 8: Specificity of Pro-Inflammatory Signaling Elicited by Caki-1 Cells Treated with Erastin

Agent/Therapeutic Design:

Caki-1 renal carcinoma cells are treated with either control (DMSO) or various doses of Erastin (e.g. 0.098, 0.195, 0.391, 0.781, 1.563, 3.125, 6.25, 12.5 and 25 μM) in the presence or absence of 1 μM Ferrostatin (Selleckchem; Houston, Tex.) for 24 hours prior to co-culture with THP1-Dual cells for an additional 24 hours. Subsequently, the THP1 supernatant is assessed for NFKB or IRF reporter activity. Caki-1 cells are acquired from ATCC and THP1-Dual cells acquired from InvivoGen (San Diego, Calif.). Both cell types are cultured in 96-well plates for the duration of the assay. The specificity of induction of NFKB signaling elicited by Erastin-treated Caki-1 cells is assessed by its reversal by concomitant ferrostatin treatment of Caki-1 cells.

### Example 9: Specificity of Pro-Inflammatory Signaling Elicited by Caki-1 Renal Carcinoma Cells Treated with RSL3

Agent/Therapeutic Design:

Caki-1 renal carcinoma cells are treated with either control (DMSO) or various doses of RSL3 (e.g. 0.002, 0.005, 0.014, 0.041, 0.123, 0.370, 1.111, 3.333 and 10 μM) in the presence or absence of 1 μM Ferrostatin for 24 hours prior to co-culture with THP1-Dual cells for an additional 24 hours. Subsequently, the THP1 supernatant is assessed for NFKB or IRF reporter activity. Caki-1 cells are acquired from ATCC and THP1-Dual cells acquired from InvivoGen (San Diego, Calif.). Both cell types are cultured in 96-well plates for the duration of the assay. The specificity of induction of NFKB signaling elicited by RSL3-treated Caki-1 cells is assessed by its reversal by concomitant ferrostatin treatment of Caki-1 cells.

### Example 10: Specificity of Pro-Inflammatory Signaling Elicited by A20 Cells Treated with RSL3

Agent/Therapeutic Design:

A20 lymphoma cells are treated with either control (DMSO) or various doses of RSL3 (e.g. 0.002, 0.005, 0.014, 0.041, 0.123, 0.370, 1.111, 3.333 and 10 μM) in the presence or absence of 1 μM Ferrostatin for 24 hours prior to co-culture with THP1-Dual cells for an additional 24 hours. Subsequently, the THP1 supernatant is assessed for NFKB or IRF reporter activity. A20 cells are acquired from ATCC and THP1-Dual cells are acquired from InvivoGen (San Diego, Calif.). Both cell types are cultured in 96-well plates for the duration of the assay. The specificity of induction of NFKB signaling elicited by RSL3-treated A20 cells is assessed by its reversal by concomitant ferrostatin treatment of A20 cells.

### Example 11: Induction of an Anti-Tumor/Pro-Inflammatory Response In Vivo by RSL3 Treatment of A20 Lymphoma Tumor Xenografts

Agent/Therapeutic Design:

BALB/C mice are subcutaneously injected with 5×10⁶ A20 lymphoma cells. Mice are dosed intratumorally by injection with Vehicle or RSL3 when tumors reach a median size of 150-200 mm³. 48 hours later immunophenotyping is performed on tumor infiltrating cells, lymphocytes, and splenocytes to characterize the recruitment and activation status of myeloid and lymphoid cells. Immunophenotyping is performed either by immunohistochemistry/immunofluorescence staining of tumor sections, or by first dissociating the tumor into single cell suspensions and then subjecting the cells to flow cytometry (J Vis Exp., 2015, (98): 52657; J Natl Cancer Inst. 2015 Feb. 3; 107(3); Cancer Discov. 2012 July; 2(7):608-23.). Compared to vehicle, an induction of a pro-inflammatory response by RSL3 treatment is assessed by an increased recruitment of monocytes, macrophages and T cells into the tumor microenvironment. Further, anti-tumor immune responses are assessed by determining increases in activation markers in macrophages, (MHCII and CD80) CD11+CD103+Dendritic cells (MHCII) and in both CD4 and CD8 T cells (Ki67 and CD69) without concomitant activation of CD4+FoxP3+T-regulatory cells. In addition, the inhibition of tumor growth by RSL3 is assessed by measurements of tumor size over a three-week period or until tumors reach a maximum size of 2000 mm³.

### Example 12: Induction of a Systemic Anti-Tumor/Pro-Inflammatory Response In Vivo by Local RSL3 Treatment of A20 Lymphoma Tumor Xenografts

Agent/Therapeutic Design:

BALB/C mice are subcutaneously injected with 5×10⁶ A20 lymphoma cells at two different sites within the body. Mice are dosed intratumorally with Vehicle or RSL3 at one tumor site when tumors reach a median size of 150-200 mm³. The inhibition of tumor growth by RSL3 is assessed by measurements of tumor size of both the treated and non-treated (contralateral) tumor over a three-week period or until tumors reach a maximum size of 2000 mm³. In addition, engagement of systemic adaptive immune responses is assessed by analyzing the tumor infiltrating lymphocytes (TILs) within the contralateral tumor site. Therapeutically relevant adaptive immune responses in the contralateral tumor are assessed by either quantitative increases in T effector cell number (FoxP3-CD4+ T cells, CD8+ T cells) or by increased activation status of T cells (CD69, ki67), macrophages (MHCII and CD80) or CD11+CD103+Dendritic cells (MHCII).

### Example 13: Use of RSL3 to Treat Renal Cell Carcinoma in a Human Clinical Trial

Agent/Therapeutic Design:

RSL3 induces cell death of renal cell carcinoma cells in a manner that causes pro-inflammatory signaling. Using methods of the present disclosure that involve using RSL3, the present Example is expected to provide evidence of the therapeutic effects of RSL3.

A randomized controlled trial (RCT) is conducted to evaluate the safety and efficacy of RSL3 following multiple infusions of RSL3 alone or in combination with nivolumab, compared to multiple infusions of nivolumab, in the treatment of renal carcinoma patients that have failed 1 or 2 anti-angiogenic therapy regimens.

One hundred patients with advanced RCC that failed anti-angiogenic therapies are randomized to receive either RSL3 alone (10 mg daily), nivolumab alone (3 mg/kg every 2 weeks) or RSL3 in combination with nivolumab. Patients are required to have a Karnosky Performance Score (KPS) of at least 70%, have no evidence of brain metastases, have not received prior treatment with nivolumab and do not have active autoimmune disease or medical conditions requiring systemic immunosuppression. The KPS ranking runs from 100 to 0, where 100 is no evidence of disease, and 0 is death, and is used to evaluate a patient's ability to survive chemotherapy.

Tumor assessments begin on week 8 following commencement of therapy and continue every 8 weeks thereafter for the first year and every 12 weeks until progression or treatment discontinuation. RSL3 efficacy is evaluated by assessment of overall survival rates.

### Example 14: Induction of Anti-Tumor Immune Response In Vivo by a Combination of Piperazine Erastin and Anti-CTLA4 Antibody (9D9) Treatment of B16.BL6 Melanoma Tumor Xenografts

Agent/Therapeutic Design:

C57/BL6 mice are subcutaneously injected with 1×10⁵ B16.BL6 melanoma cells. Mice are dosed intratumorally with Vehicle, Piperazine Erastin (40 mg/kg, i.p.), anti-CTLA4 antibody 9D9 (10 mg/kg, i.p.), or a combination of Piperazine Erastin and 9D9. Mice are dosed when tumors reach a median size of 150-200 mm³. 48 hours later immunophenotyping is performed on tumor infiltrating cells, lymphocytes and splenocytes to characterize the recruitment and activation status of myeloid and lymphoid cells. Compared to vehicle, an induction of a maximal pro-inflammatory response by the combination of Piperazine Erastin and 9D9 treatment is assessed by an increased recruitment of CD3+ T cells into the tumor microenvironment compared to either treatment alone. In addition, the maximal inhibition of tumor growth by the combination therapy compared to either treatment alone is assessed by measurements of tumor size over a three-week period or until tumors reach a maximum size of 2000 mm³.

### Example 15: A Method of Screening for Compounds that Induce Pro-Inflammatory Ferroptosis

Agent/Therapeutic Design:

Caki-1 renal carcinoma cells in 384-well format are exposed to test compounds from a chemical screening library in the absence or presence of a ferroptosis inhibitor (ferrostatin, B-Mercaptoethanol or an iron chelator) for 24-48 hours. Subsequently, THP1 dual cells are co-cultured with the treated Caki-1 cells. 24 hours after addition of THP1-Dual cells, supernatants are assessed for NFKB or IRF reporter activity. Compounds that induce NFKB or IRF reporter activity in the absence of a ferroptosis inhibitor, but do not induce NFKB or IRF reporter activity in the presence of a ferroptosis inhibitor are selected as pro-inflammatory compounds.

### Example 16: A Method of Testing the Inflammatory Nature of Materials Originating from Cells Treated with Ferroptosis Inducers

Agent/Therapeutic Design:

Caki-1 renal carcinoma cells are exposed to Erastin or RSL3 (or other ferroptosis inducers) and conditioned medium is collected 24-48 hours later. Subsequently, conditioned medium is extracted with organic solvent followed by HPLC fractionation. Specifically, conditioned medium is extracted using ethyl acetate, concentrated, and fractionated by polarity. Alternatively, conditioned medium is subjected to size exclusion chromatography with collection of fractions. Specifically, conditioned medium is applied to a size exclusion column and fractionated on FPLC. Collected fractions are applied to THP1-Dual cells for 24 hours with subsequent assessment of reporter activity. Positive hit fractions are confirmed by their ability to induce NFKB or IRF activity in THP1 Dual cells. Positive hit fractions are further characterized by mass spectrometry (large molecules) or NMR (small molecules) to identify particular compounds with inflammatory activity. The inflammatory nature of the individual compounds or species are tested by the addition of synthetic or recombinant forms of such compounds or species to THP1 Dual Cells followed by measurement of NFKB or IRF activity, as described above.

### Example 17: Specificity of Pro-Inflammatory Signaling Elicited by HT1080 Fibrosarcoma Cells Treated with Erastin

Agent/Therapeutic Design:

HT1080 fibrosarcoma cells were treated with various doses of Erastin (e.g. 0.8, 0.16, 0.31, 0.63, 1.25, 2.5, 5, 10 or 20 μM) alone or in combination with a ferroptosis inhibitor (1 μM Ferrostatin-1, 1 μM Liproxstatin-1, 100 μM Trolox, 25 μM β-Mercaptoethanol or 100 μM Deferoxamine) for 24 hours prior to co-culture with THP1-Dual cells for an additional 24 hours. Ferrostatin-1 and Liproxstatin-1 were purchased from Selleckchem (Houston, Tex.) and dissolved in DMSO. Trolox was purchased from Cayman Chemical Company Inc and resuspended in DMSO. Deferoxamine mesylate was purchased from Sigma-Aldrich and resuspended in water. β-Mercaptoethanol was purchased from Life Technologies. Subsequently, the THP1 supernatant was assessed for NFKB activity. HT1080 cells were acquired from ATCC and THP1-Dual cells were acquired from InvivoGen (San Diego, Calif.). Both cell types were cultured in 96-well plates for the duration of the assay.

Results:

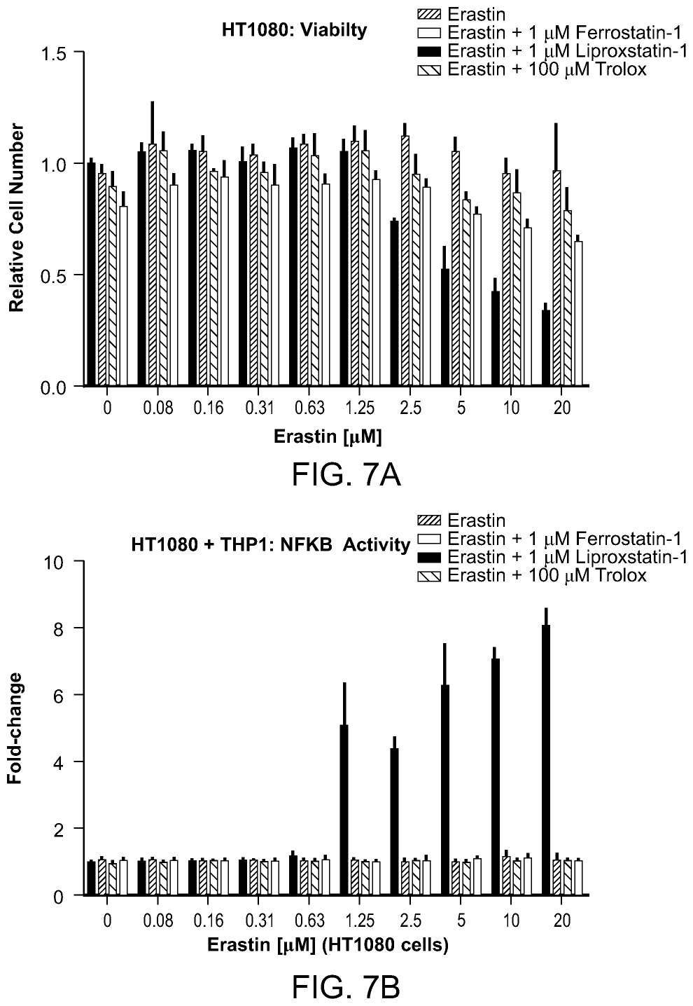

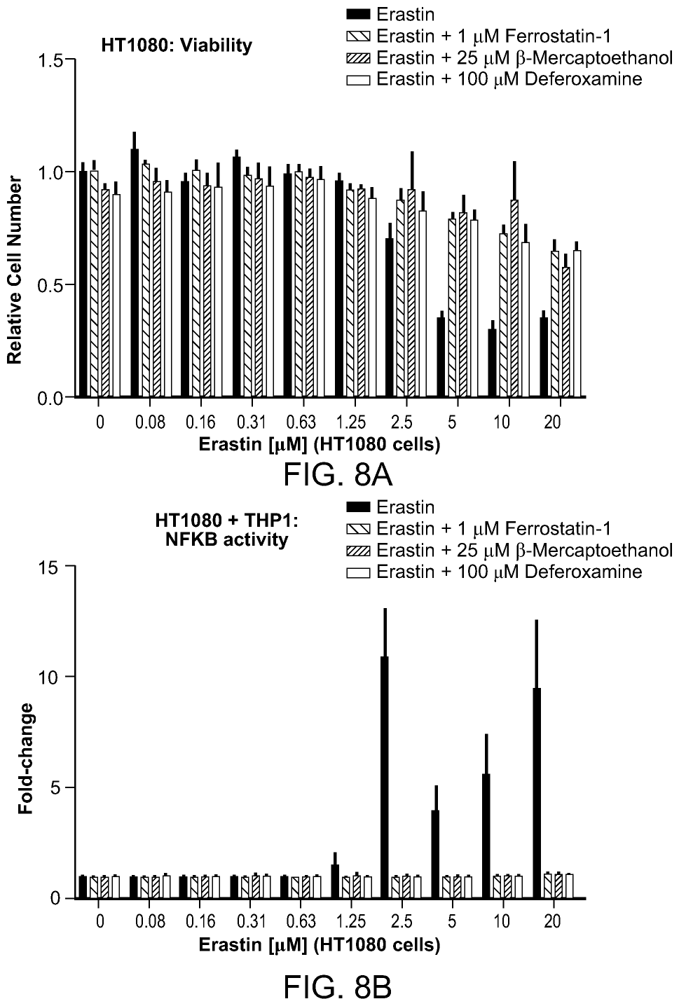

As shown in FIGS. 7A and 8A, Erastin treatment of HT1080 fibrosarcoma cells decreased viability of the cells in a dose dependent manner, and this decreased viability was attenuated by each of the ferroptosis inhibitors. As shown in FIGS. 7B and 8B, Erastin treatment of HT1080 fibrosarcoma cells increased NFKB activity in THP1 cells in a dose dependent manner, and this increased NFKB activity was abrogated by each of the ferroptosis inhibitors. These results demonstrate that cell death plays a role in the induction of NFKB signaling elicited by Erastin-treated HT1080 cells.

### Example 18: Knockdown of ACSL4 and CARS Genes Inhibits Erastin-Mediated Cell Death in HT1080 Fibrosarcoma Cells

Agent/Therapeutic Design:

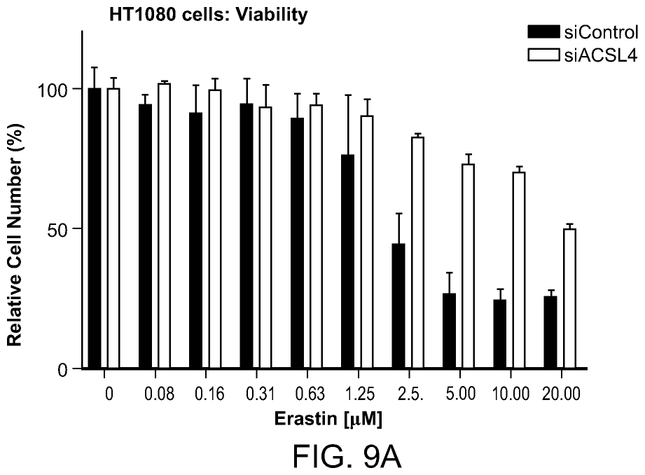

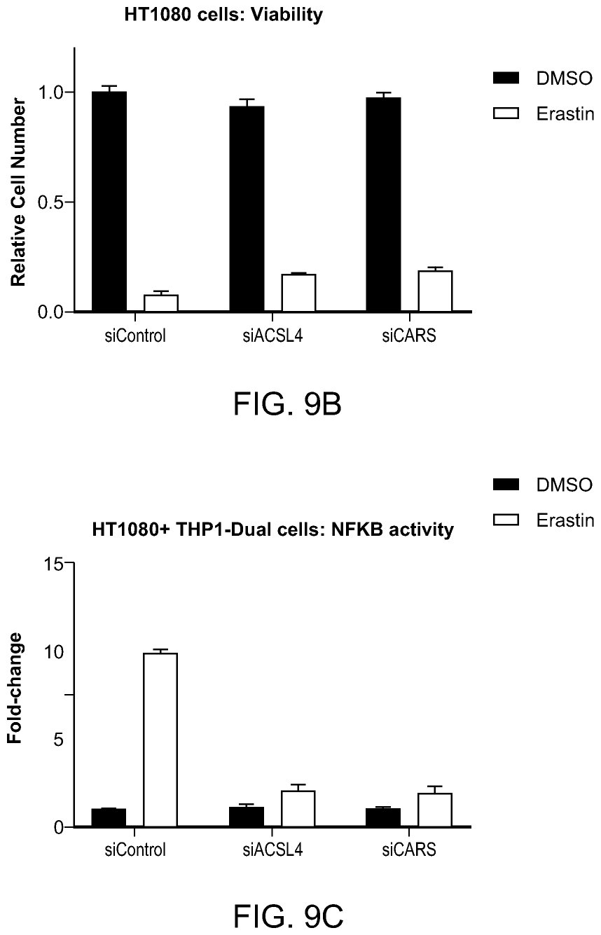

HT1080 cells (5,000 cells/well) were reverse transfected in 96-well format using DharmaFECT I transfection reagent (Catalog #T-2001) and control siRNA (Dharmacon Catalog #D-001810-10-05) or siRNA [37.5 nM] targeting ACSL4 (FIG. 9A, Dharmacon Catalog #L-009364-00-005) or a pool of siRNAs (FIG. 9B/C) against ACSL4 (Thermo Fisher Silencer Select Catalog #'s: s5001, s5001, s5002) or CARS (Thermo Fisher Silencer Select Catalog #'s: 52404, s2405, s2406). 48 hours post-transfection, cell culture medium was replaced by fresh medium containing variours concentrations of Erastin (FIG. 9A) or a fixed concentration of Erastin (10 μM, FIG. 9B/C). In addition, 50,000 reporter THP1-dual cells were added to some plates (FIG. 9C). 24 hours later, HT1080 cell viability was measured (FIG. 9A/B) or THP1 superntatant was assessed for NFKB activity (FIG. 9C). HT1080 cells were acquired from ATCC and THP1-Dual cells were acquired from InvivoGen (San Diego, Calif.).

Results:

The ACSL4 gene encodes Long-chain-fatty-acid-CoA ligase 4, an acyl-CoA synthetase that controls the level of arachidonic acid in cells, and is involved in the regulation of cell death. The CARS gene encodes cysteinyl-tRNA synthetase. Knockdown of CARS has been shown to inhibit erastin-induced ferroptosis by preventing the induction of lipid reactive oxygen species. See Hayano et al., 2016, Cell Death Differ. 23(2): 270-278. As shown in FIGS. 9A and 9B, genetic knockdown of either ACLS4 or CARS in HT1080 cells partially rescues viability of HT1080 cells cultured in the presence of Erastin. In addition, genetic knockdown of either ACLS4 or CARS in HT1080 cells abrogates NFKB activity in monocytes co-cultured with Erastin-treated HT1080 cells (FIG. 9C). These results demonstrate that cell death plays a role in the induction of NFKB signaling elicited by Erastin-treated HT1080 cells and that the absence of specific intracellular proteins reduces the pro-inflammatory nature of ferroptosis.

### Example 19: Specificity of Pro-Inflammatory Signaling Elicited by A20 Cells Treated with a GPX4 Inhibitor (RSL3, ML162 or ML210)

Agent/Therapeutic Design:

A20 lymphoma cells were treated with various doses (e.g. 0.002, 0.005, 0.014, 0.041, 0.123, 0.370, 1.111, 3.333 and 10 μM) of a GPX4 inhibitor (RSL3, ML162 or ML210) in the presence or absence of 1 μM Ferrostatin-1 for 24 hours. ML162 was purchased from Cayman Chemical Company Inc and resuspended in DMSO. ML210 was purchased from Sigma-Aldrich and resuspended in DMSO. A20 lymphoma cells were also treated with DMSO as a negative control. After treatment with DMSO or a GPX4 inhibitor for 24 hours, the A20 lymphoma cells were co-cultured with THP1-Dual cells for an additional 24 hours. Subsequently, the THP1 supernatant was assessed for NFKB reporter activity. A20 cells were acquired from ATCC and THP1-Dual cells were acquired from InvivoGen (San Diego, Calif.). Both cell types were cultured in 96-well plates for the duration of the assay.

Results:

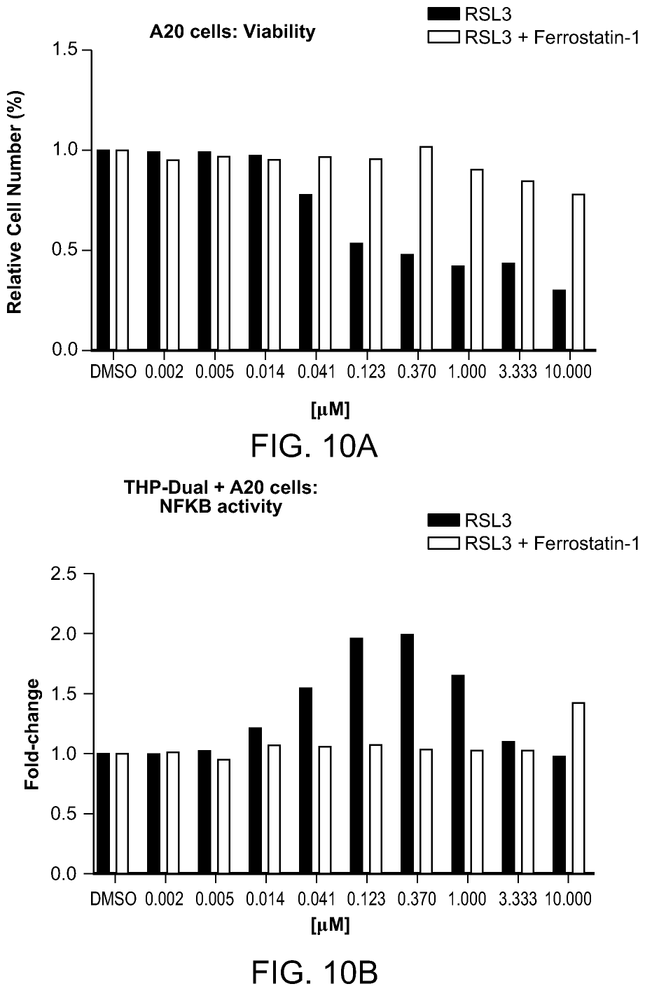

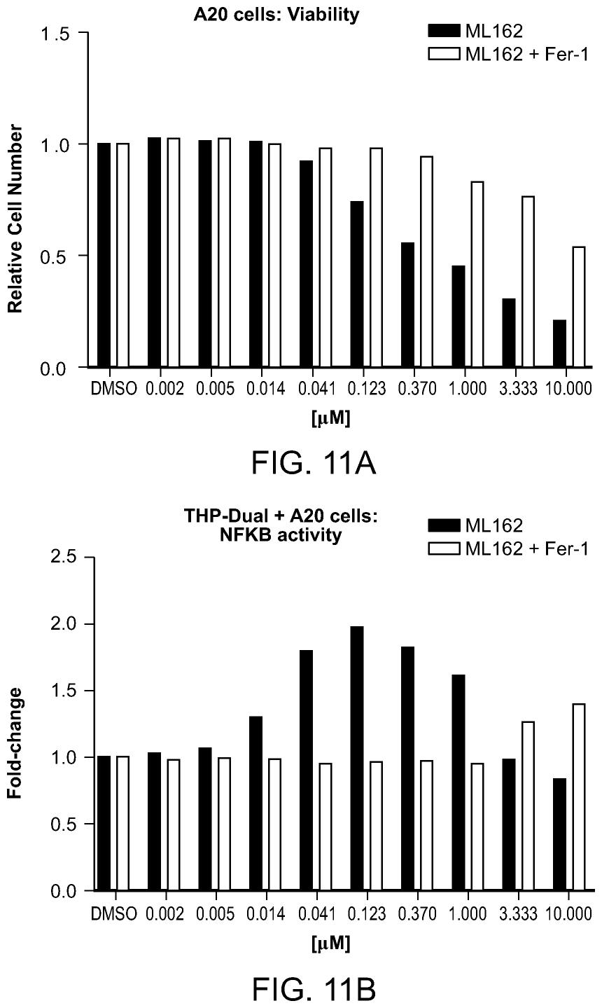

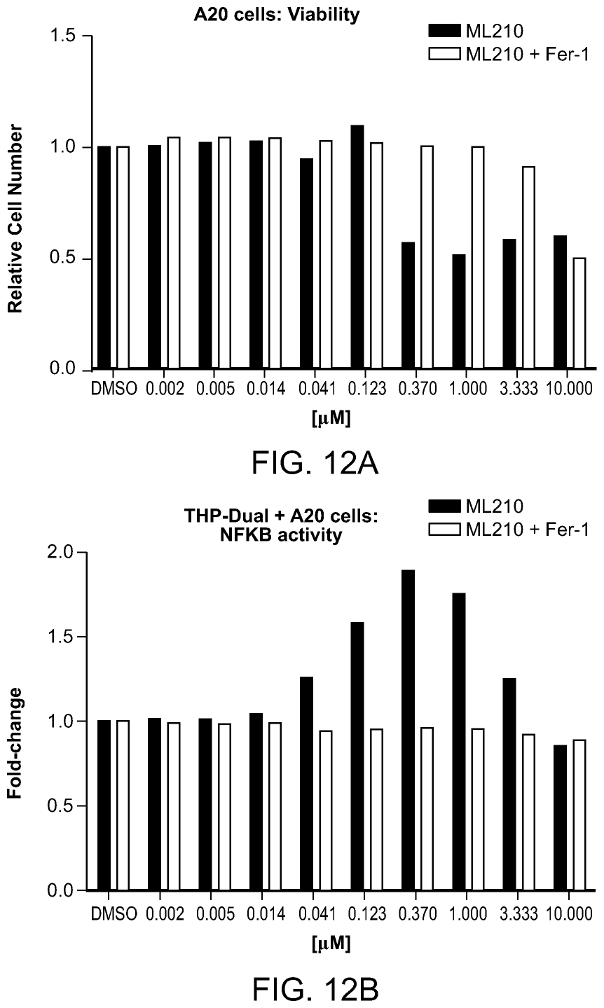

As shown in FIGS. 10A, 11A and 12A, treatment of A20 lymphoma cells with each of the GPX4 inhibitors (RSL3, ML162 or ML210) decreased viability of the cells in a dose dependent manner, and this decreased viability was attenuated by the ferroptosis inhibitor Ferrostatin-1. As shown in FIGS. 10B, 11B and 12B, GPX4 inhibitor treatment of A20 lymphoma cells increased NFKB activity in THP1 cells in a dose dependent manner, and this increased NFKB activity was attenuated by the ferroptosis inhibitor Ferrostatin-1. These results demonstrate that cell death plays a role in the induction of NFKB signaling elicited by GPX-4 inhibitor-treated A20 lymphoma cells.

### Example 20: Specificity of Pro-Inflammatory Signaling Elicited by Caki-1 Renal Carcinoma Cells Treated with a GPX4 Inhibitor (RSL3 or ML162)

Agent/Therapeutic Design:

Caki-1 renal carcinoma cells were treated with either control (DMSO) or various doses (e.g. 0.002, 0.005, 0.014, 0.041, 0.123, 0.370, 1.111, 3.333 and 10 μM) of a GPX4 inhibitor (RSL3 or ML162) in the presence or absence of 1 μM Ferrostatin for 24 hours prior to co-culture with THP1-Dual cells for an additional 24 hours. Subsequently, the THP1 supernatant was assessed for NFKB reporter activity. Caki-1 cells were acquired from ATCC and THP1-Dual cells acquired from InvivoGen (San Diego, Calif.). Both cell types were cultured in 96-well plates for the duration of the assay.

Results:

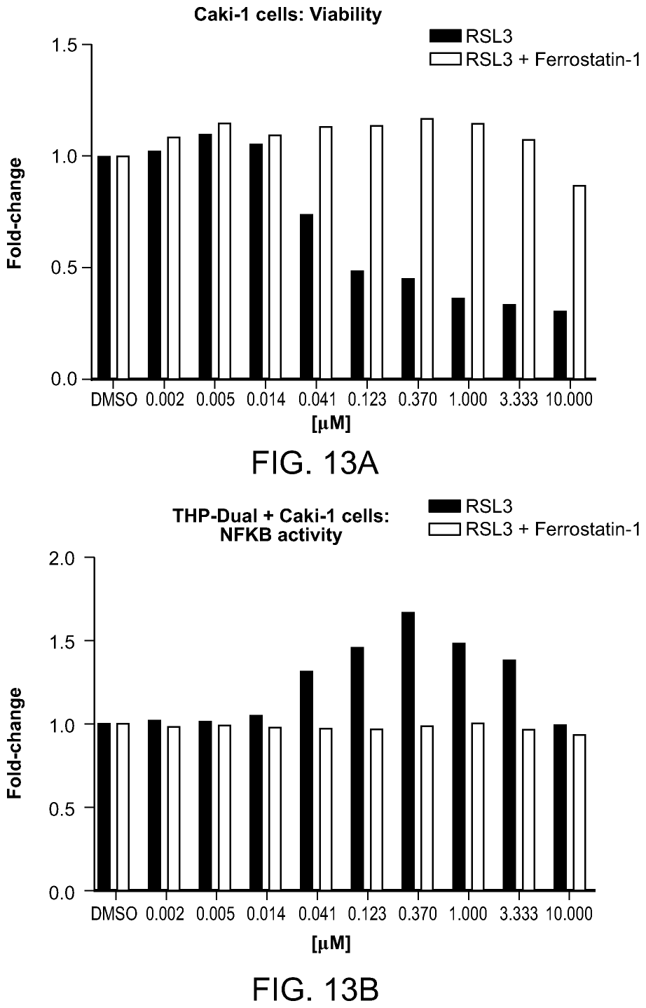

As shown in FIGS. 13A and 14A, treatment of Caki-1 renal carcinoma cells with a GPX4 inhibitor (RSL3 or ML162) decreased viability of the cells in a dose dependent manner, and this decreased viability was attenuated by the ferroptosis inhibitor Ferrostatin-1. As shown in FIGS. 13B and 14B, treatment of Caki-1 renal carcinoma cells with RSL3 or ML162 increased NFKB activity in THP1 cells in a dose dependent manner, and this increased NFKB activity was attenuated by the ferroptosis inhibitor Ferrostatin-1. These results demonstrate that cell death plays a role in the induction of NFKB signaling elicited by GPX-4 inhibitor-treated Caki-1 renal carcinoma cells.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

Each reference, patent, and patent application referred to in the instant application is hereby incorporated by reference in its entirety as if each reference were noted to be incorporated individually.

1. A method of increasing immune activity in an immune cell, comprising:
(i) contacting a target cell with an agent that induces iron-dependent cellular disassembly and
(ii) exposing the immune cell to the target cell that has been contacted with the agent or to postcellular signaling factors produced by the target cell that has been contacted with the agent, in an amount sufficient to increase the immune activity in the immune cell relative to an immune cell in the absence of contacting the target cell with the agent, wherein the agent that induces iron-dependent cellular disassembly is selected from the group consisting of an inhibitor of antiporter system Xc⁻, an inhibitor of GPX4, and a statin.

2. A method of increasing the level or activity of NFkB in an immune cell, comprising:
(i) contacting a target cell with an agent that induces iron-dependent cellular disassembly and
(ii) exposing the immune cell to the target cell that has been contacted with the agent or to postcellular signaling factors produced by the target cell that has been contacted with the agent, in an amount sufficient to increase the level or activity of NFkB in the immune cell relative to an immune cell in the absence of contacting the target cell with the agent, wherein the agent that induces iron-dependent cellular disassembly is selected from the group consisting of an inhibitor of antiporter system Xc⁻, an inhibitor of GPX4, and a statin.

3. A method of increasing the level or activity of interferon regulatory factor (IRF) or Stimulator of Interferon Genes (STING) in an immune cell, comprising: (i) contacting a target cell with an agent that induces iron-dependent cellular disassembly and (ii) exposing the immune cell to the target cell that has been contacted with the agent or to postcellular signaling factors produced by the target cell that has been contacted with the agent, in an amount sufficient to increase the level or activity of IRF or STING in the immune cell relative to an immune cell in the absence of contacting the target cell with the agent, wherein the agent that induces iron-dependent cellular disassembly is selected from the group consisting of an inhibitor of antiporter system Xc⁻, an inhibitor of GPX4, and a statin.

4. A method of increasing the level or activity of a pro-immune cytokine in an immune cell, comprising: (i) contacting a target cell with an agent that induces iron-dependent cellular disassembly and (ii) exposing the immune cell to the target cell that has been contacted with the agent or to postcellular signaling factors produced by the target cell that has been contacted with the agent, in an amount sufficient to increase the level or activity of the pro-immune cytokine in the immune cell relative to an immune cell in the absence of contacting the target cell with the agent, wherein the agent that induces iron-dependent cellular disassembly is selected from the group consisting of an inhibitor of antiporter system Xc⁻, an inhibitor of GPX4, and a statin.

5. The method of any one of claims 1 to 4, wherein the iron-dependent cellular disassembly is ferroptosis.

6. The method of any one of claims 1 to 4, wherein the inhibitor of antiporter system Xc⁻is erastin or a derivative or analog thereof.

7. The method of claim 6, wherein the erastin or derivative or analog thereof has the following formula: 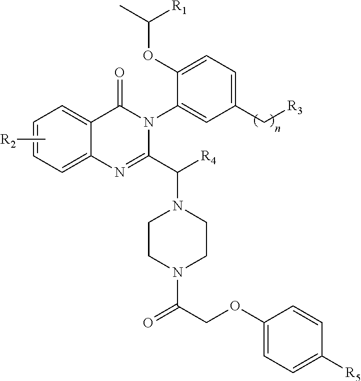
or pharmaceutically acceptable salts or esters thereof, wherein
R₁ is selected from the group consisting of H, C₁₋₄ alkyl, C₁₋₄ alkoxy, hydroxy, and halogen;
R₂ is selected from the group consisting of H, halo, and C₁₋₄ alkyl;
R₃ is selected from the group consisting of H, C₁₋₄ alkyl, C₁₋₄ alkoxy, 5-7 membered heterocycloalkyl, and 5-6 membered heteroaryl;
R₄ is selected from the group consisting of H and C₁₋₄ alkyl;
R₅ is halo; 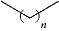
is optionally substituted with ═O; and
n is an integer from 0-4.

8. The method of claim 6, wherein the analog of erastin is PE or IKE.

9. The method of any one of claims 1 to 4, wherein the inhibitor of GPX4 is selected from the group consisting of (1S,3R)-RSL3 or a derivative or analog thereof, ML162, DPI compound 7, DPI compound 10, DPI compound 12, DPI compound 13, DPI compound 17, DPI compound 18, DPI compound 19, FIN56, and FINO2.

10. The method of claim 9, wherein the RSL3 derivative or analog is a compound represented by Structural Formula (I): 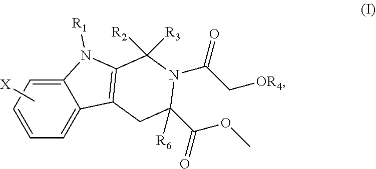
or an enantiomer, optical isomer, diastereomer, N-oxide, crystalline form, hydrate, or pharmaceutically acceptable salt thereof, wherein
R₁, R₂, R₃, and R₆ are independently selected from H, C₁₋₈alkyl, C₁₋₈alkoxy, C₁₋₈aralkyl, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkoxy, aralkyl, carbocyclic, heterocyclic, aryl, heteroaryl, acyl, alkylsulfonyl, and arylsulfonyl is optionally substituted with at least one substituent;
R₄ and R₅ are independently selected from H₁ C₁₋₈alkyl, C₁₋₈alkoxy, 3- to 8-membered carbocyclic, 3- to 8-membered heterocyclic, 3- to 8-membered aryl, or 3-to 8-membered heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether is optionally substituted with at least one substituent;
R⁷ is selected from H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R⁸ is selected from H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; and
X is 0-4 substituents on the ring to which it is attached.

11. The method of claim 9, wherein the RSL3 derivative or analog is a compound represented by Structural Formula (II): 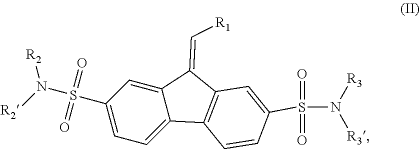
or an N-oxide, crystalline form, hydrate, or pharmaceutically acceptable salt thereof; wherein:
R₁ is selected from the group consisting of H, OH, and —(OCH₂CH₂)ₓOH;
X is an integer from 1 to 6; and
R₂, R₂′, R₃, and R₃′ independently are selected from the group consisting of H, C₃₋₈cycloalkyl, and combinations thereof, or R₂ and R₂′ may be joined together to form a pyridinyl or pyranyl and R₃ and R₃′ may be joined together to form a pyridinyl or pyranyl.

12. The method of claim 9, wherein the RSL3 derivative or analog is a compound represented by Structural Formula (III): 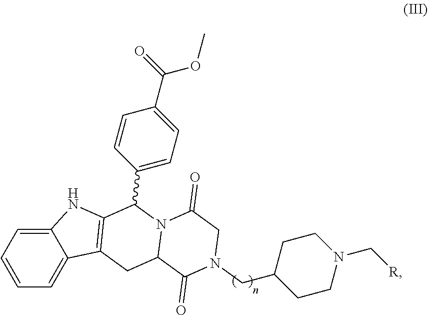
or a stereoisomer thereof, or a pharmaceutically acceptable salt thereof; wherein: n is 2, 3 or 4; and R is a substituted or unsubstituted C₁-C₆ alkyl group, a substituted or unsubstituted C₃-C₁₀ cycloalkyl group, a substituted or unsubstituted C₂-C₈ heterocycloalkyl group, a substituted or unsubstituted C₆-C₁₀ aromatic ring group, or a substituted or unsubstituted C₃-C₈ heteroaryl ring group; wherein the substitution means that one or more hydrogen atoms in each group are substituted by the following groups selected from the group consisting of: halogen, cyano, nitro, hydroxy, C₁-C₆ alkyl, halogenated C₁-C₆ alkyl, C₁-C₆ alkoxy, halogenated C₁-C₆ alkoxy, COOH (carboxy), COOC₁-C₆ alkyl, OCOC₁-C₆ alkyl.

13. The method of any one of claims 1 to 4, wherein the statin is selected from the group consisting of atorvastatin, fluvastatin, lovastatin, pitavastatin, pravastatin, rosuvastatin, cerivastatin and simvastatin.

14. The method of any one of claims 1 to 4, wherein the immune cell is a macrophage, monocyte, dendritic cell, T cell, CD4+ cell, CD8+ cell, or CD3+ cell.

15. The method of any one of claims 1 to 4, wherein the immune cell is a THP-1 cell.

16. The method of any one of claims 1 to 15, wherein the method is carried out in vitro or ex vivo.

17. The method of any one of claims 1 to 15, wherein the method is carried out in vivo.

18. The method of any one of claims 1 to 15, wherein step (i) is carried out in vitro and step (ii) is carried out in vivo.

19. A method of increasing immune activity in a cell, tissue or subject, the method comprising administering to the cell, tissue or subject an agent that induces iron-dependent cellular disassembly in an amount sufficient to increase the immune activity relative to a cell, tissue or subject that is not treated with the agent that induces iron-dependent cellular disassembly.

20. The method of claim 19, wherein the subject is in need of an increased immune activity.

21. The method of claim 19 or 20, wherein the agent that induces iron-dependent cellular disassembly is administered in an amount sufficient to increase in the cell, tissue or subject one or more of: the level or activity of NFkB, the level or activity of interferon regulatory factor (IRF) or Stimulator of Interferon Genes (STING), the level or activity of macrophages, the level or activity of monocytes, the level or activity of dendritic cells, the level or activity of T cells, the level or activity of CD4+, CD8+ or CD3+ cells, and the level or activity of a pro-immune cytokine.

22. A method of increasing the level or activity of NFkB in a cell, tissue or subject, comprising administering to the cell, tissue or subject an agent that induces iron-dependent cellular disassembly in an amount sufficient to increase the level or activity of NFkB relative to a cell, tissue or subject that is not treated with the agent that induces iron-dependent cellular disassembly.

23. The method of claim 22, wherein the subject is in need of an increased level or activity of NFkB.

24. The method of claim 22 or 23, wherein the level or activity of NFkB is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

25. A method of increasing the level or activity of interferon regulatory factor (IRF) or Stimulator of Interferon Genes (STING) in a cell, tissue or subject, comprising administering to the cell, tissue or subject an agent that induces iron-dependent cellular disassembly in an amount sufficient to increase the level or activity of IRF or STING relative to a cell, tissue or subject that is not treated with the agent that induces iron-dependent cellular disassembly.

26. The method of claim 25, wherein the subject is in need of an increased level or activity of IRF or STING.

27. The method of claim 25 or 26, wherein the level or activity of IRF or STING is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

28. A method of increasing the level or activity of macrophages, monocytes, dendritic cells or T cells in a tissue or subject, comprising administering to the tissue or subject an agent that induces iron-dependent cellular disassembly in an amount sufficient to increase the level or activity of macrophages, monocytes, dendritic cells or T cells relative to a tissue or subject that is not treated with the agent that induces iron-dependent cellular disassembly.

29. The method of claim 28, wherein the subject is in need of an increased level or activity of macrophages, monocytes, dendritic cells or T cells.

30. The method of claim 28, wherein the level or activity of macrophages, monocytes, dendritic cells, or T cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the agent that induces iron-dependent cellular disassembly.

31. A method of increasing the level or activity of CD4+, CD8+, or CD3+ cells in a tissue or subject, comprising administering to the tissue or subject an agent that induces iron-dependent cellular disassembly in an amount sufficient to increase the level or activity of CD4+, CD8+, or CD3+ cells relative to a tissue or subject that is not treated with the agent that induces iron-dependent cellular disassembly.

32. The method of claim 31, wherein the subject is in need of an increased level or activity of CD4+, CD8+, or CD3+ cells.

33. The method of claim 31 or 32, wherein the level or activity of CD4+, CD8+, or CD3+ cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the agent that induces iron-dependent cellular disassembly.

34. A method of increasing the level or activity of a pro-immune cytokine in a cell, tissue or subject, comprising administering to the cell, tissue or subject an agent that induces iron-dependent cellular disassembly in an amount sufficient to increase the level or activity of the pro-immune cytokine relative to a cell, tissue or subject that is not treated with the agent that induces iron-dependent cellular disassembly.

35. The method of claim 34, wherein the subject is in need of an increased level or activity of a pro-immune cytokine.

36. The method of claim 34 or 35, wherein the level or activity of the pro-immune cytokine is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

37. The method of any one of claims 1 to 36, further comprising, before the administering, evaluating the cell, tissue or subject for one or more of: the level or activity of NFkB; the level or activity of macrophages; the level or activity of monocytes; the level or activity of dendritic cells; the level or activity of CD4+ cells, CD8+ cells, or CD3+ cells; the level or activity of T cells; and the level or activity of a pro-immune cytokine.

38. The method of any one of claims 1 to 36, further comprising, after the administering, evaluating the cell, tissue or subject for one or more of: the level or activity of NFkB; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine.

39. The method of any one of claims 4 and 34 to 38, wherein the pro-immune cytokine is selected from IFN-α, IL-1, IL-12, IL-18, IL-2, IL-15, IL-4, IL-6, TNF-α, IL-17 and GMCSF.

40. A method of treating a subject in need of increased immune activity, the method comprising administering to the subject an agent that induces iron-dependent cellular disassembly in an amount sufficient to increase the immune activity in the subject.

41. The method of any one of claims 19 to 40, wherein the subject has an infection.

42. The method of claim 41, wherein the infection is a chronic infection.

43. The method of claim 42, wherein the chronic infection is selected from HIV infection, HCV infection, HBV infection, HPV infection, Hepatitis B infection, Hepatitis C infection, EBV infection, CMV infection, TB infection, and infection with a parasite.

44. The method of any one of claims 1 to 39, wherein the cell or tissue is a cancer cell or cancerous tissue.

45. The method of any one of claims 19 to 40, wherein the subject has cancer.

46. The method of claim 45, wherein the cancer is selected from melanoma, renal cell carcinoma, non-small cell lung cancer, non-squamous cell lung cancer, urothelial carcinoma, Hodgkin's lymphoma, head and neck squamous cell carcinoma, hepatocellular carcinoma, colorectal cancer, gastric adenocarcinoma, gastric esophageal junction adenocarcinoma, and Merkel cell carcinoma.

47. The method of any one of claims 1 to 46, wherein the iron-dependent cellular disassembly is ferroptosis.

48. A method of treating a cancer in a subject in need thereof, comprising administering to the subject, in combination (a) an immunotherapeutic and (b) an agent that induces iron-dependent cellular disassembly, thereby treating the cancer in the subject.

49. The method of claim 48, wherein the agent that induces iron-dependent cellular disassembly is administered to the subject in an amount effective to increase immune response in the subject.

50. The method of claim 48, wherein the immunotherapeutic is selected from the group consisting of a Toll-like receptor (TLR) agonist, a cell-based therapy, a cytokine, a cancer vaccine, and an immune checkpoint modulator of an immune checkpoint molecule.

51. The method of claim 50, wherein the TLR agonist is selected from Coley's toxin and Bacille Calmette-Guérin (BCG).

52. The method of 50, wherein the immune checkpoint molecule is selected from CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA.

53. The method of claim 50, wherein the immune checkpoint molecule is a stimulatory immune checkpoint molecule and the immune checkpoint modulator is an agonist of the stimulatory immune checkpoint molecule.

54. The method of claim 50, wherein the immune checkpoint molecule is an inhibitory immune checkpoint molecule and the immune checkpoint modulator is an antagonist of the inhibitory immune checkpoint molecule.

55. The method of any one of claims 50 to 54, wherein the immune checkpoint modulator is selected from a small molecule, an inhibitory RNA, an antisense molecule, and an immune checkpoint molecule binding protein.

56. The method of claim 50, wherein the immune checkpoint molecule is PD-1 and the immune checkpoint modulator is a PD-1 inhibitor.

57. The method of claim 56, wherein the PD-1 inhibitor is selected from pembrolizumab, nivolumab, pidilizumab, SHR-1210, MEDI0680R01, BBg-A317, TSR-042, REGN2810 and PF-06801591.

58. The method of claim 50, wherein the immune checkpoint molecule is PD-L1 and the immune checkpoint modulator is a PD-L1 inhibitor.

59. The method of claim 58, wherein the PD-L1 inhibitor is selected from durvalumab, atezolizumab, avelumab, MDX-1105, AMP-224 and LY3300054.

60. The method of claim 50, wherein the immune checkpoint molecule is CTLA-4 and the immune checkpoint modulator is a CTLA-4 inhibitor.

61. The method of claim 60, wherein the CTLA-4 inhibitor is selected from ipilimumab, tremelimumab, JMW-3B3 and AGEN1884.

62. The method of any one of claims 48 to 61, wherein the agent that induces iron-dependent cellular disassembly is administered before or concurrently with administration of the immunotherapeutic.

63. The method of any one of claims 48 to 61, wherein the agent that induces iron-dependent cellular disassembly is administered after administration of the immunotherapeutic.

64. The method of any one of claims 48 to 63, wherein a response of the cancer to treatment is improved relative to a treatment with the immunotherapeutic alone.

65. The method of claim 64, wherein the response is improved, by at least 5%, at least 10%, at least 15%, at least 20%, at least 30%, at least 40%, at least 50%, at least 60%, at least 70%, or at least 80% relative to treatment with the immunotherapeutic alone.

66. The method of claim 64 or 65, wherein the response comprises any one or more of reduction in tumor burden, reduction in tumor size, inhibition of tumor growth, achievement of stable cancer in a subject with a progressive cancer prior to treatment, increased time to progression of the cancer, and increased time of survival.

67. The method of any one of claims 48 to 66, wherein the agent that induces iron-dependent cellular disassembly and the immunotherapeutic act synergistically.

68. The method of any one of claims 48 to 67, wherein the cancer is a cancer responsive to an immune checkpoint therapy.

69. The method of any one of claims 48 to 68, wherein the cancer is selected from a carcinoma, sarcoma, lymphoma, melanoma, and leukemia.

70. The method of any one of claims 48 to 68, wherein the cancer is selected from melanoma, renal cell carcinoma, non-small cell lung cancer, non-squamous cell lung cancer, urothelial carcinoma, Hodgkin's lymphoma, head and neck squamous cell carcinoma, hepatocellular carcinoma, colorectal cancer, gastric adenocarcinoma, gastric esophageal junction adenocarcinoma, and Merkel cell carcinoma.

71. The method of any one of claims 48 to 68, wherein the cancer is renal cell carcinoma.

72. The method of any one of claims 19 to 71, wherein the subject is human.

73. The method of any one of claims 1 to 72, wherein the agent that induces iron-dependent cellular disassembly is selected from the group consisting of an inhibitor of antiporter system Xc⁻, an inhibitor of GPX4, and a statin.

74. The method of claim 73, wherein the inhibitor of antiporter system Xc⁻ is erastin or a derivative or analog thereof.

75. The method of claim 74, wherein the erastin or derivative or analog thereof has the following formula: 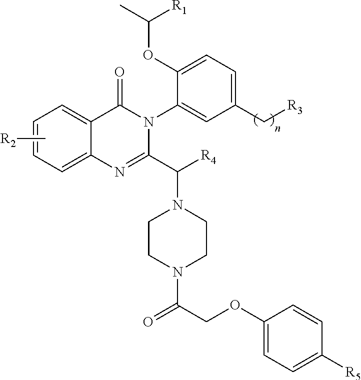
or pharmaceutically acceptable salts or esters thereof, wherein
R₁ is selected from the group consisting of H, C₁₋₄ alkyl, C₁₋₄ alkoxy, hydroxy, and halogen;
R₂ is selected from the group consisting of H, halo, and C₁₋₄ alkyl;
R₃ is selected from the group consisting of H, C₁₋₄ alkyl, C₁₋₄ alkoxy, 5-7 membered heterocycloalkyl, and 5-6 membered heteroaryl;
R₄ is selected from the group consisting of H and C₁₋₄ alkyl;
R₅ is halo; 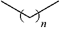
is optionally substituted with ═O; and
n is an integer from 0-4.

76. The method of claim 74, wherein the analog of erastin is PE or IKE.

77. The method of claim 73, wherein the inhibitor of GPX4 is selected from the group consisting of (1S,3R)-RSL3 or a derivative or analog thereof, ML162, DPI compound 7, DPI compound 10, DPI compound 12, DPI compound 13, DPI compound 17, DPI compound 18, DPI compound 19, FIN56, and FINO2.

78. The method of claim 77, wherein the RSL3 derivative or analog is a compound represented by Structural Formula (I): 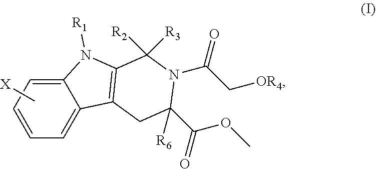
or an enantiomer, optical isomer, diastereomer, N-oxide, crystalline form, hydrate, or pharmaceutically acceptable salt thereof, wherein
R₁, R₂, R₃, and R₆ are independently selected from H, C₁₋₈alkyl, C₁₋₈alkoxy, C₁₋₈aralkyl, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkoxy, aralkyl, carbocyclic, heterocyclic, aryl, heteroaryl, acyl, alkylsulfonyl, and arylsulfonyl is optionally substituted with at least one substituent;
R₄ and R₅ are independently selected from H₁ C₁₋₈alkyl, C₁₋₈alkoxy, 3- to 8-membered carbocyclic, 3- to 8-membered heterocyclic, 3- to 8-membered aryl, or 3-to 8-membered heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether is optionally substituted with at least one substituent;
R⁷ is selected from H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R⁸ is selected from H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; and
X is 0-4 substituents on the ring to which it is attached.

79. The method of claim 77, wherein the RSL3 derivative or analog is a compound represented by Structural Formula (II): 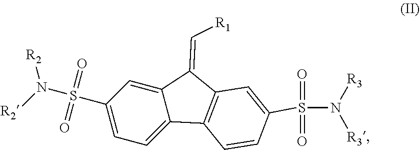
or an N-oxide, crystalline form, hydrate, or pharmaceutically acceptable salt thereof; wherein:
R₁ is selected from the group consisting of H, OH, and —(OCH₂CH₂)ₓOH;
X is an integer from 1 to 6; and
R₂, R₂′, R₃, and R₃′ independently are selected from the group consisting of H, C₃₋₈cycloalkyl, and combinations thereof, or R₂ and R₂′ may be joined together to form a pyridinyl or pyranyl and R₃ and R₃′ may be joined together to form a pyridinyl or pyranyl.

80. The method of claim 77, wherein the RSL3 derivative or analog is a compound represented by Structural Formula (III): 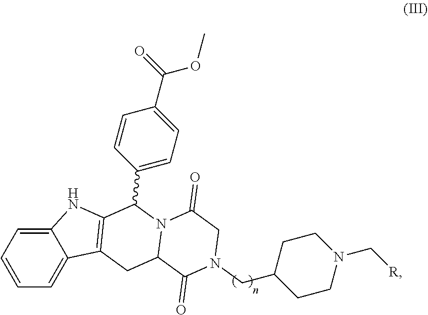
or a stereoisomer thereof, or a pharmaceutically acceptable salt thereof; wherein:
n is 2, 3 or 4; and R is a substituted or unsubstituted C₁-C₆ alkyl group, a substituted or unsubstituted C₃-C₁₀ cycloalkyl group, a substituted or unsubstituted C₂-C₈ heterocycloalkyl group, a substituted or unsubstituted C₆-C₁₀ aromatic ring group, or a substituted or unsubstituted C₃-C₈ heteroaryl ring group; wherein the substitution means that one or more hydrogen atoms in each group are substituted by the following groups selected from the group consisting of: halogen, cyano, nitro, hydroxy, C₁-C₆ alkyl, halogenated C₁-C₆ alkyl, C₁-C₆ alkoxy, halogenated C₁-C₆ alkoxy, COOH (carboxy), COOC₁-C₆ alkyl, OCOC₁-C₆ alkyl.

81. The method of claim 73, wherein the statin is selected from the group consisting of atorvastatin, fluvastatin, lovastatin, pitavastatin, pravastatin, rosuvastatin, cerivastatin and simvastatin.

82. The method of any one of claims 1 to 72, wherein the agent that induces iron-dependent cellular disassembly is selected from the group consisting of sorafenib or a derivative or analog thereof, sulfasalazine, glutamate, BSO, DPI2, cisplatin, cysteinase, silica based nanoparticles, CCI4, ferric ammonium citrate, trigonelline and brusatol.

83. The method of any one of claims 1 to 72, wherein the agent that induces iron-dependent cellular disassembly has one or more of the following characteristics:
(a) induces iron-dependent cellular disassembly of a target cell in vitro and subsequent activation of an immune response in a co-cultured cell;
(b) induces iron-dependent cellular disassembly of a target cell in vitro and subsequent activation of co-cultured macrophages, e.g., RAW264.7 macrophages;
(c) induces iron-dependent cellular disassembly of a target cell in vitro and subsequent activation of co-cultured monocytes, e.g., THP-1 monocytes;
(d) induces iron-dependent cellular disassembly of a target cell in vitro and subsequent activation of co-cultured bone marrow-derived dendritic cells (BMDCs);
(e) induces iron-dependent cellular disassembly of a target cell in vitro and subsequent increase in levels or activity of NFkB, IRF and/or STING in a co-cultured cell;
(f) induces iron-dependent cellular disassembly of a target cell in vitro and subsequent increase in levels or activity of a pro-immune cytokine in a co-cultured cell; and
(g) induces iron-dependent cellular disassembly of a target cell in vitro and subsequent activation of co-cultured CD4+ cells, CD8+ cells and/or CD3+ cells.

84. The method of any one of claims 1 to 72, wherein the agent that induces iron-dependent cellular disassembly is targeted to a cancer cell.

85. A method of screening for an immunostimulatory agent, the method comprising:
(a) providing a plurality of test agents (e.g., a library of test agents);
(b) evaluating each of the plurality of test agents for the ability to induce iron-dependent cellular disassembly;
(c) selecting as a candidate immunostimulatory agent a test agent that induces iron-dependent cellular disassembly; and
(d) evaluating the candidate immunostimulatory agent for the ability to stimulate an immune response.

86. The method of claim 85, wherein the evaluating step (b) comprises contacting cells or tissue with each of the plurality of test agents.

87. The method of claim 85, wherein the evaluating step (b) comprises administering each of the plurality of test agents to an animal.

88. The method of claim 86, wherein the evaluating step (b) further comprises measuring the level or activity of a marker selected from the group consisting of lipid peroxidation, reactive oxygen species (ROS), isoprostanes, malondialdehyde (MDA), iron, glutathione peroxidase 4 (GPX4), prostaglandin-endoperoxide synthase 2 (PTGS2), cyclooxygenase-2 (COX-2), and glutathione (GSH) in the cells or tissue contacted with the test agent.

89. The method of claim 85, wherein the evaluating step (b) further comprises comparing the level or activity of the marker in the cells or tissue contacted with the test agent to the level or activity of the marker in a control cell or tissue that has not been contacted with the test agent.

90. The method of claim 85, wherein the evaluating step (d) comprises evaluating the test agent that induces iron-dependent cellular disassembly for immunostimulatory activity in vitro.

91. The method of claim 85, wherein the evaluating step (d) comprises measuring immune response in an animal.

92. The method of any one of claims 85 to 91, wherein an increase in the level or activity of a marker selected from the group consisting of lipid peroxidation, isoprostanes, reactive oxygen species (ROS), iron, PTGS2 and COX-2, or a decrease in the level or activity of a marker selected from the group consisting of GPX4, MDA and GSH indicates that the test agent is an agent that induces iron-dependent cellular disassembly.

93. The method of claim 85, wherein evaluating the candidate immunostimulatory agent comprises culturing an immune cell together with cells contacted with the selected candidate immunostimulatory agent or exposing an immune cell to postcellular signaling factors produced by cells contacted with the selected candidate immunostimulatory agent and measuring the level or activity of NFKB, IRF or STING in the immune cell.

94. The method of claim 85, wherein evaluating the candidate immunostimulatory agent comprises culturing T cells together with cells contacted with the selected candidate immunostimulatory agent or exposing T cells to postcellular signaling factors produced by cells contacted with the selected candidate immunostimulatory agent and measuring the activation and proliferation of the T cells.

95. A method of identifying an immunostimulatory agent, the method comprising:
(a) contacting a cell with an agent that induces iron-dependent cellular disassembly in an amount sufficient to induce iron-dependent cellular disassembly in the cell;
(b) isolating one or more postcellular signaling factors produced by the cell after contact with the agent that induces iron-dependent cellular disassembly; and
(c) assaying the one or more postcellular signaling factors for the ability to stimulate immune response.

96. The method of claim 95, wherein the method further comprises selecting a test agent that stimulates immune response.

97. The method of claim 95, wherein the method further comprises detecting a marker of iron-dependent cellular disassembly in the cell.

98. The method of claim 95, wherein the method further comprises:
i) measuring the level of the one or more postcellular signaling factors produced by the cell after contact with the agent that induces iron-dependent cellular disassembly;
ii) comparing the level of the one or more postcellular signaling factors produced by the cell after contact with the agent that induces iron-dependent cellular disassembly to the level of the one or more test agents in a control cell that is not treated with the agent that induces iron-dependent cellular disassembly; and
iii) selecting postcellular signaling factors that exhibit increased levels in the cell contacted with the agent that induces iron-dependent cellular disassembly relative to the control cell to generate the one or more postcellular signaling factors for assaying in step (c).

99. The method of claim 98, wherein the control cell is treated with an agent that induces a cell death that is not iron-dependent cellular disassembly.

100. The method of claim 95, wherein the assaying comprises administering the one or more postcellular signaling factors to an animal and measuring immune response in the animal.

101. The method of claim 95, wherein the assaying comprises treating an immune cell with the one or more postcellular signaling factors and measuring the level or activity of NFKB activity in the immune cell.

102. The method of claim 95, wherein the assaying comprises treating T cells with the one or more postcellular signaling factors and measuring the activation or proliferation of the T cells.

103. The method of claim 95, wherein the assaying comprises contacting an immune cell with the one or more postcellular signaling factors and measuring the level or activity of NFκB, IRF or STING in the immune cell.

104. The method of claim 93 or 103, wherein the immune cell is a THP-1 cell.
